SEQUENCE LISTING (3 sequences)

SEQ ID NO: 1 - RNA, 25 nt, Artificial Sequence
  RNA template-loop-primer for biotin-dUTP, Stavudine-TP or AZT-TP extension reactions
uuuucuacgcguaguuuucuacgcg

SEQ ID NO: 2 - RNA, 25 nt, Artificial Sequence
  RNA template-loop-primer for Carbovir-TP or Ganciclovir-TP extension reactions
uuuucuccgcguaguuuucuacgcg

SEQ ID NO: 3 - RNA, 25 nt, Artificial Sequence
  RNA template-loop-primer for Tenofovir-DP extension reactions
uuuucuucgcguaguuuucuacgcg

(19) United States
(12) Patent Application Publication
(10) Pub. No.: US 2023/0172961 A1
(43) Pub. Date: Jun. 8, 2023

(54) THERAPEUTICS FOR COVID-19

(71) Applicants: Jingyue JU, Englewood Cliffs, NJ (US); Steffen JOCKUSCH, New York, NY (US); Chuanjuan TAO, Fort Lee, NJ (US); Minchen CHIEN, Tenafly, NJ (US); James J. RUSSO, New York, NY (US); Shiv KUMAR, Belle Mead, NJ (US); Xiaoxu LI, New York, NY (US); Xuanting WANG, Braintree, MA (US)

(72) Inventors: Jingyue Ju, Englewood Cliffs, NJ (US); Steffen Jockusch, New York, NY (US); Chuanjuan Tao, Fort Lee, NJ (US); Minchen Chien, Tenafly, NJ (US); James J. Russo, New York, NY (US); Shiv Kumar, Belle Mead, NJ (US); Xiaoxu Li, New York, NY (US); Xuanting Wang, Braintree, MA (US)

(73) Assignee: The Trustees of Columbia University in the City of New York, New York, NY (US)

(21) Appl. No.: 17/796,629

(22) PCT Filed: Jan. 29, 2021
(86) PCT No.: PCT/US2021/015672

§ 371 Date: Jul. 29, 2022

Related U.S. Application Data
(60) Provisional application No. 63/130,303, filed on Dec. 23, 2020.
(60) Provisional application No. 63/070,231, filed on Aug. 25, 2020.
(60) Provisional application No. 63/063,171, filed on Aug. 7, 2020.
(60) Provisional application No. 63/013,432, filed on Apr. 21, 2020.
(60) Provisional application No. 63/001,155, filed on Mar. 27, 2020.
(60) Provisional application No. 62/991,508, filed on Mar. 18, 2020.
(60) Provisional application No. 62/988,798, filed on Mar. 12, 2020.
(60) Provisional application No. 62/984,190, filed on Mar. 2, 2020.
(60) Provisional application No. 62/983,272, filed on Feb. 28, 2020.
(60) Provisional application No. 62/972,803, filed on Feb. 11, 2020.
(60) Provisional application No. 62/968,011, filed on Jan. 30, 2020.
(60) Provisional application No. 62/967,452, filed on Jan. 29, 2020.

(51) Int. Cl. A61K 31/7072; A61K 31/7076; A61K 31/708; A61K 31/7068; A61K 45/06

(52) U.S. Cl. CPC A61K 31/7072; A61K 31/708; A61K 31/7068; A61K 31/7076; A61K 45/06

(57) ABSTRACT

This invention relates to the use of nucleoside, nucleotide and other compounds which are inhibitors or terminators of viral RNA dependent RNA polymerases or inhibitors of exonucleases as antiviral agents. These antiviral agents can be used alone or in combination with other polymerase or exonuclease inhibitors, helicase inhibitors, HCV NS5A inhibitors, HIV integrase inhibitors and HCV NS3-4A and other protease inhibitors to treat viral infections such as SARS-CoV-2, the causative agent of the COVID-19 infection.

This application claims priority of U.S. Provisional Application Nos. 62/967,452, filed Jan. 29, 2020, 62/968,011, filed Jan. 30, 2020, 62/972,803, filed Feb. 11, 2020, 62/983,272, filed Feb. 28, 2020, 62/984,190, filed Mar. 2, 2020, 62/988,798, filed Mar. 12, 2020, 62/991,508, filed Mar. 18, 2020, 63/001,155, filed Mar. 27, 2020, 63/013,432, filed Apr. 21, 2020, 63/063,171, filed Aug. 7, 2020, 63/070,231, filed Aug. 25, 2020, and 63/130,303, filed Dec. 23, 2020, the contents of each of which are hereby incorporated by reference.

Throughout this application, various publications and patents are referenced. Full citations for these references may be found at the end of the specification immediately preceding the claims. The disclosures of these publications and patents in their entirety are hereby incorporated by reference into this application to more fully describe the state of the art to which this invention pertains.

### TECHNOLOGY FIELD

This invention relates to the use of nucleoside, nucleotide and other compounds which are inhibitors or terminators of viral RNA dependent RNA polymerases or inhibitors of exonucleases as antiviral agents. These antiviral agents can be used alone or in combination with other polymerase or exonuclease inhibitors, helicase inhibitors, HCV NS5A inhibitors, HIV integrase inhibitors and HCV NS3-4A and other protease inhibitors to treat viral infections such as SARS-CoV-2, the causative agent of the COVID-19 infection.

### BACKGROUND OF THE INVENTION

SARS-CoV-2, the virus responsible for the COVID-19 pandemic, is a new member of the subgenus Sarbecovirus, in the Orthocoronavirinae subfamily, but is distinct from MERS-CoV and SARS-CoV (Zhu et al 2020). The virus was first isolated from the lower respiratory tracts of patients with pneumonia, sequenced and visualized by electron microscopy (Zhu et al 2020). Coronaviruses are single strand RNA viruses, sharing properties with other single-stranded RNA viruses such as hepatitis C virus (HCV), West Nile virus, Marburg virus, HIV virus, Ebola virus, dengue virus, and rhinoviruses. In particular, coronaviruses and HCV are both positive-sense single-strand RNA viruses (Zumla et al 2016, Dustin et al 2016), and thus have a similar replication mechanism requiring an RNA-dependent RNA polymerase (RdRp).

The coronavirus life cycle has been described (Zumla et al 2016, FIG. 1). Briefly, the virus enters the cell by endocytosis, is uncoated, and ORF1a and ORF1b of the positive strand RNA are translated to produce nonstructural protein precursors, including a cysteine protease and a serine protease; these further cleave the precursors to form mature, functional helicase and RdRp. A replication-transcription complex is then formed, which is responsible for making more copies of the RNA genome via a negative-sense RNA intermediate, as well as the structural and other proteins encoded by the viral genome. The viral RNA is packaged into viral coats in the endoplasmic reticulum-Golgi intermediate complex, after which exocytosis results in release of viral particles for subsequent infectious cycles. Potential inhibitors have been designed to target nearly every stage of this process (Zumla et al 2016). However, despite decades of research, few effective drugs are currently approved to treat serious coronavirus infections such as SARS, MERS, and COVID-19.

SARS-CoV-2 proteins. SARS-CoV-2 has an ~29.9 kb RNA genome encoding 4 structural proteins (Spike (S), Membrane (M), Nucleocapsid (N) and Envelope (E)), a large number of non-structural proteins (nsps 1-16) and a number of smaller accessory proteins (https://www.genetex.com/MarketingMaterial/Index/SARS-CoV-2_Genome_and_Proteome). Several of the nsps cooperate to form the replication complex (FIG. 2). These consist of the RNA-dependent RNA polymerase (RdRp, nsp12) and its cofactors nsp7 and nsp8; the ssRNA binding protein nsp9; the proofreading 3′-5′ exonuclease nsp14 (which also has a methyltransferase-based capping activity) and its cofactor nsp10; and the multifunctional helicase and capping enzyme nsp13. Other proteins are involved in capping (nsp16), endonuclease (nsp15) and protease (nsp3, nsp5) functions, vesicle formation and inhibition of viral factor transport to lysosomes (nsp6), viral assembly and entry into vesicles (nsp4), or host protein interactions (nsp1, nsp2 and nsp4). Any and all of these may be considered druggable targets. Drugs that inhibit one or preferably a combination of these protein activities are described in this invention, with particular emphasis on those that inhibit the RdRp or its complex, the exonuclease, the helicase or the protease activities.

One of the most important druggable targets for coronaviruses is the RdRp. This polymerase is highly conserved at the protein level among different positive sense RNA viruses, e.g., coronaviruses and HCV, and shares common structural features in these viruses (te Velthuis 2014). Like RdRps in other viruses, the coronavirus enzyme is highly error-prone (Selisko et al 2018) which might increase its ability to accept modified nucleotide analogues as substrates. Nucleotide and nucleoside analogues that inhibit polymerases are an important group of anti-viral agents (McKenna et al 1989, Oberg 2006, Eltahla et al 2015, De Clercq & Li 2016).

Based on our analysis of hepatitis C virus and coronavirus replication, and the molecular structures and activities of viral inhibitors, we reasoned that the FDA-approved hepatitis C drug EPCLUSA (Sofosbuvir/Velpatasvir) should inhibit coronaviruses, including SARS-CoV-2 (Ju et al 2020a). Sofosbuvir is a pyrimidine nucleotide analogue prodrug with a hydrophobic masked phosphate group enabling it to enter infected eukaryotic cells, and then be converted into its active triphosphate form by cellular enzymes (FIG. 3). In this activated form, it inhibits the HCV RdRp NS5B (Kayali & Schmidt 2014, Sofia et al 2010). The activated drug (2′-F,Me-UTP) binds in the active site of the RdRp, where it is incorporated into RNA, and due to fluoro and methyl modifications at the 2′ position, inhibits further RNA chain extension, thereby halting RNA replication and stopping viral growth. It acts as an RNA polymerase inhibitor by competing with natural ribonucleotides. Velpatasvir inhibits NS5A, a key protein required for HCV replication. NS5A enhances the function of RNA polymerase NS5B during viral RNA synthesis (Gitto et al 2017, Quezada & Kane 2009).

There are many other RNA polymerase inhibitors that have been evaluated as antiviral drugs. A related purine nucleotide prodrug, Remdesivir (FIG. 4b), was developed by Gilead to treat Ebola virus infections, though not successfully, and have gone through intensive clinical trials for treating COVID-19 (Holshue et al 2020, Wang et al 2020). In contrast to Sofosbuvir

(FIG. 4a), both the 2′- and 3′-OH groups in Remdesivir (FIG. 4b) are unmodified, but a cyano group at the 1′ position in the active triphosphate form serves to inhibit the RdRp. In addition to the use of hydrophobic groups to mask the phosphate in the ProTide-based prodrug strategy (Alanazi et al 2019), as with Sofosbuvir and Remdesivir, there are other classes of nucleoside prodrugs including those based on ester derivatives of the ribose hydroxyl groups to enhance cellular delivery (De Clercq & Field 2006, Roberts et al 2008). A related prodrug analogue developed by BioCryst Pharmaceuticals, BCX4430, also known as Galidesivir (FIG. 4c), has been shown to inhibit RNA polymerases from a broad spectrum of RNA viruses, including the filoviruses (e.g., Ebola, Marburg) in rodents and Marburg virus in macaques (Warren et al 2014). Upon entry into infected cells, BCX4430 is phosphorylated, and the resulting triphosphate of the nucleoside analogue serves as an RNA chain terminator. β-D-N4-hydroxycytidine is another prodrug targeting the coronavirus polymerase and was shown to have broad spectrum activity against coronaviruses, even in the presence of intact proofreading functions (Agostini et al 2019, Sheahan et al 2020). Other drugs that have been tested for treatment of COVID-19 include Ribavirin (Hung et al 2020) and Favipiravir (Joshi et al 2021).

The replication cycle of HCV is very similar to that of the coronaviruses (Zumla et al 2016). Analyzing the structure of the active triphosphate form of Sofosbuvir (FIG. 4a) compared to that of Remdesivir (FIG. 4b), both of which have already been shown to inhibit the replication of specific RNA viruses (Sofosbuvir for HCV, Remdesivir for SARS-CoV-2), we noted in particular that the 2′-modifications in Sofosbuvir (a fluoro and a methyl group) are substantially smaller than the 1′-cyano group and the 2′-OH group in Remdesivir. The bulky cyano group in close proximity to the 2′-OH may result in steric hindrance that will impact the polymerase reaction termination efficiency of the activated form of Remdesivir. Interestingly, it was reported that, using the MERS-CoV polymerase, the triphosphate of Remdesivir was preferentially incorporated relative to ATP in solution assays (Gordon et al 2020a). Nevertheless, the active triphosphate form of Remdesivir does not cause immediate polymerase reaction termination and actually leads to delayed polymerase termination in Ebola virus and respiratory syncytial virus, likely due to its 1′-cyano group and the free 2′-OH and 3′-OH groups (Gordon et al 2020a, Tchesnokov et al 2019). Compared to the active form of Sofosbuvir (2′-fluoro-2′-methyl-UTP), two other nucleotide inhibitors with related structures were reviewed: 2′-fluoro-UTP is incorporated by polymerase, but RNA synthesis may continue past the incorporated nucleotide analogue (Fung et al 2014); 2′-C-methyl-UTP has been shown to terminate the reaction catalyzed by HCV RdRp, but proofreading mechanisms can revert this inhibition in mitochondrial DNA-dependent RNA polymerase (Arnold et al 2012). Additionally, HCV develops resistance to 2′-C-methyl-UTP due to mutations of the RdRp (Dutartre et al 2006). A computational study considered the ability of various anti-HCV drugs to dock in the active site of SARS and MERS coronavirus RdRps as potential inhibitors (Elfiky et al 2017). Using a computational approach, Elfiky predicted that Sofosbuvir, IDX-184, Ribavirin, and Remdesivir might be potent drugs against COVID-19 (Elfiky 2020a, b).

Despite extensive research efforts, there is still an unmet need for the development of effective therapeutics for COVID-19. It is therefore an object of the present invention to provide compounds, compositions, and methods for the treatment and prevention of COVID-19.

### SUMMARY OF THE INVENTION

This invention provides compositions comprising RdRp inhibitors, such as Sofosbuvir or its modified forms, coupled with NS5A inhibitors, such as Velpatasvir, to inhibit the SARS-CoV-2 polymerase reaction, based on our analysis of the biological pathways of hepatitis C and coronaviruses, the molecular structures and activities of viral inhibitors, model polymerase and SARS-CoV RdRp extension experiments described herein, and the efficacy of Sofosbuvir in inhibiting the HCV RdRp.

In some embodiments, the present invention provides a composition comprising at least one of the following compounds for the treatment of viral infection caused by viruses such as SARS-CoV-2, SARS-CoV, MERS-CoV, Marburg virus, Ebola virus and influenza virus:

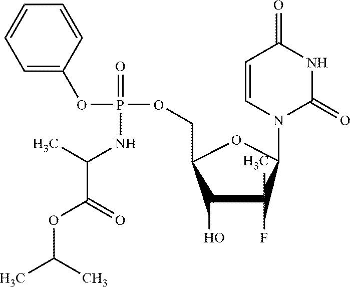

Sofosbuvir

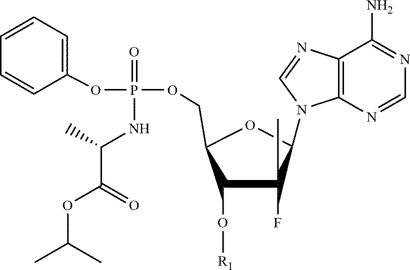

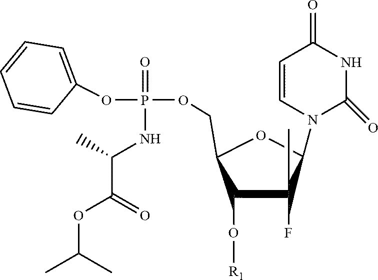

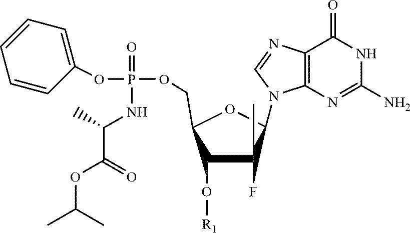

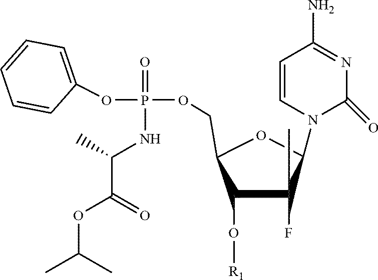

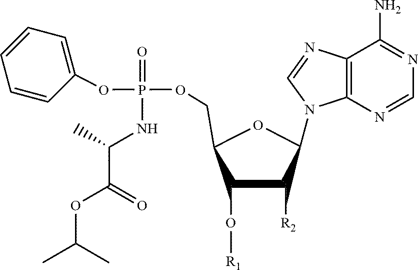

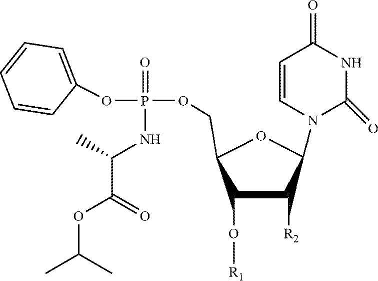

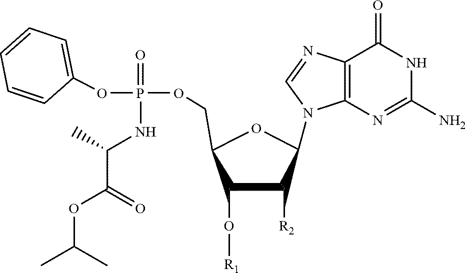

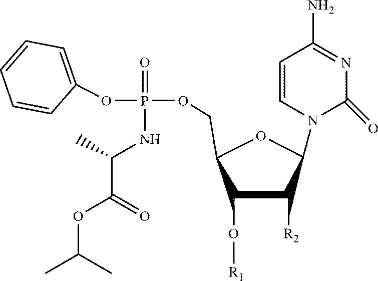

wherein R₁ is H, methyl (CH₃), ethyl (CH₂CH₃), propyl (CH₂CH₂CH₃), allyl (CH₂CH=CH₂), propargyl (CH₂C≡CH), methoxymethyl (CH₂OCH₃), methylthiomethyl (CH₂SCH₃), azidomethyl (CH₂-N₃), or other small chemical groups, as long as R₁ does not prevent the recognition of the nucleotide analogue as a substrate by the viral polymerase, and wherein R₂ is H, OH, F, or OCH₃.

In some embodiments, the natural nucleobases in these compounds may be replaced by base analogs such as 7-deaza-G, 7-deaza-A and inosine, or derivatives thereof.

In some embodiments, the present invention provides a composition comprising at least one of the following compounds for the treatment of viral infection caused by viruses such as SARS-CoV-2, SARS-CoV, MERS-CoV, hepatitis C virus, Marburg virus, Ebola virus and influenza virus:

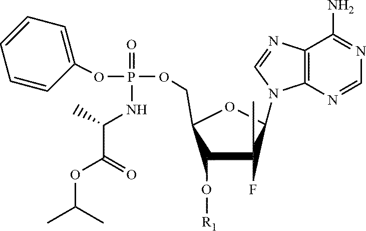

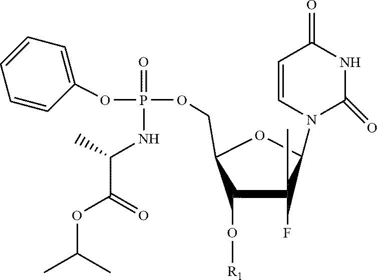

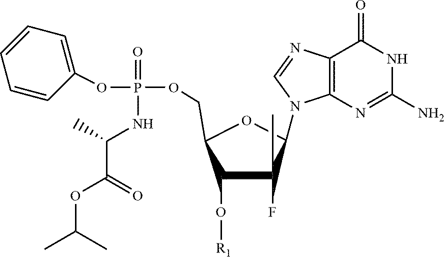

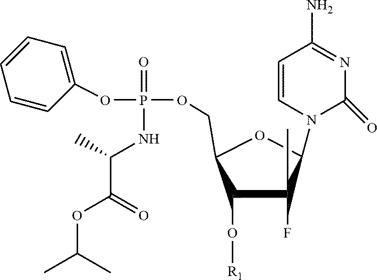

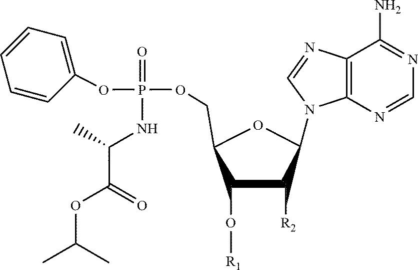

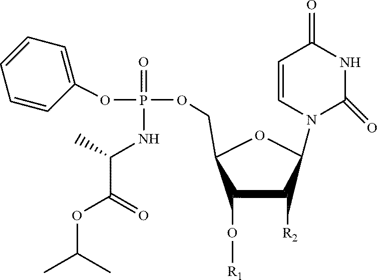

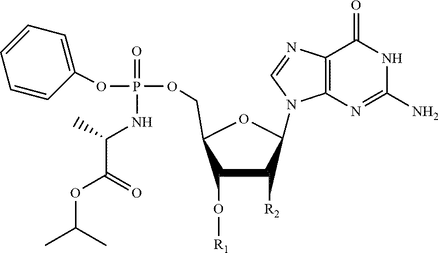

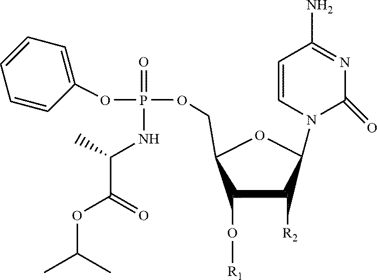

wherein R₁ is H, methyl (CH₃), ethyl (CH₂CH₃), propyl (CH₂CH₂CH₃), allyl (CH₂CH=CH₂), propargyl (CH₂C≡CH), methoxymethyl (CH₂OCH₃), methylthiomethyl (CH₂SCH₃), azidomethyl (CH₂-N₃), or other small chemical groups, as long as R₁ does not prevent the recognition of the nucleotide analogue as a substrate by the viral polymerase, and wherein R₂ is H, OH, F, or OCH₃.

In some embodiments, the natural nucleobases in these compounds may be replaced by base analogs such as 7-deaza-G, 7-deaza-A and inosine, or derivatives thereof.

In some embodiments, the present invention provides a composition comprising at least one of the following compounds for the treatment of viral infection caused by viruses such as SARS-CoV-2, SARS-CoV, MERS-CoV, Marburg virus, Ebola virus and influenza virus:

EPCLUSA (Sofosbuvir/Velpatasvir), Sofosbuvir/Daclatasvir,

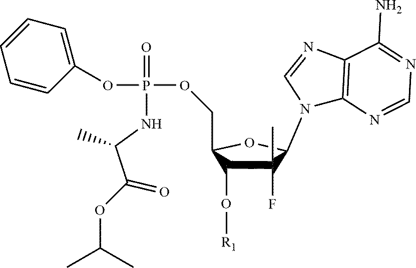

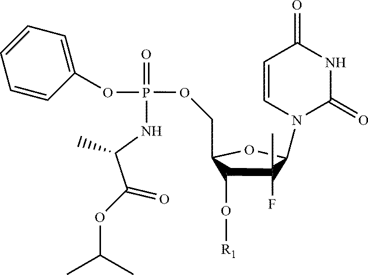

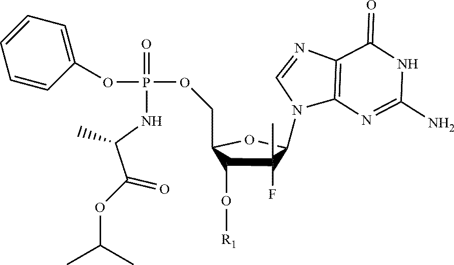

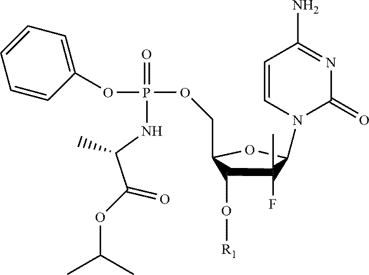

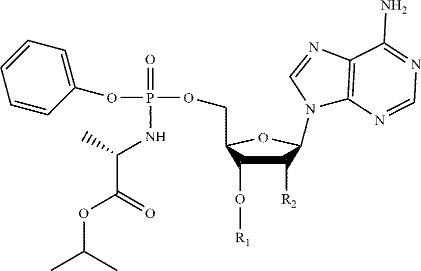

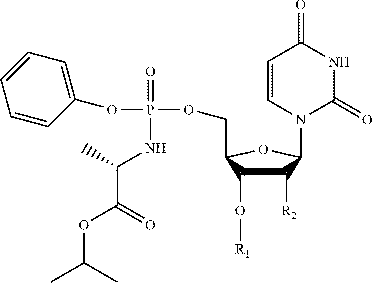

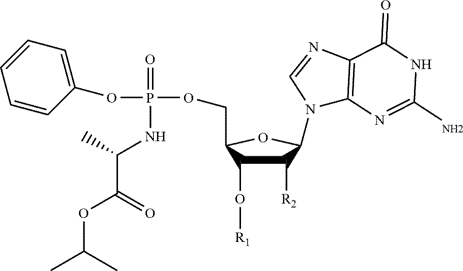

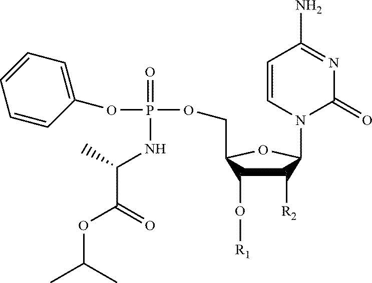

wherein R₁ is H, methyl (CH₃), ethyl (CH₂CH₃), propyl (CH₂CH₂CH₃), allyl (CH₂CH=CH₂), propargyl (CH₂C≡CH), methoxymethyl (CH₂OCH₃), methylthiomethyl (CH₂SCH₃), azidomethyl (CH₂-N₃), or other small chemical groups, as long as R₁ does not prevent the recognition of the nucleotide analogue as a substrate by the viral polymerase, and wherein R₂ is H, OH, F, or OCH₃.

In some embodiments, the natural nucleobases in these compounds may be replaced by base analogs such as 7-deaza-G, 7-deaza-A and inosine, or derivatives thereof.

In some embodiments, the present invention provides a composition comprising at least one of the following compounds for the treatment of viral infection caused by viruses such as SARS-CoV-2, SARS-CoV, MERS-CoV, hepatitis C virus, Marburg virus, Ebola virus and influenza virus:

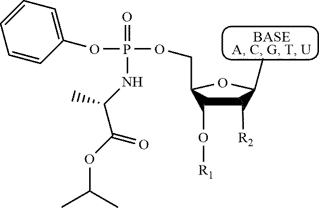

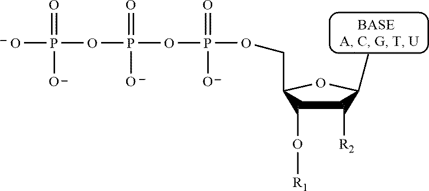

wherein R₁ is H, methyl, or other small chemical groups, as long as R₁ does not prevent the recognition of the nucleotide analogue as a substrate by the viral polymerase, wherein R₂ is OH, F, H, or -O-ester such as i-butyl ester and valyl ester, wherein BASE is A, C, G, T, U or derivatives thereof, and wherein the compounds depicted on the left are prodrugs of the active forms of the compounds depicted on the right.

In some embodiments, the present invention provides a composition comprising at least one of the following compounds for the treatment of viral infection caused by viruses such as SARS-CoV-2, SARS-CoV, MERS-CoV, hepatitis C virus, Marburg virus, Ebola virus and influenza virus:

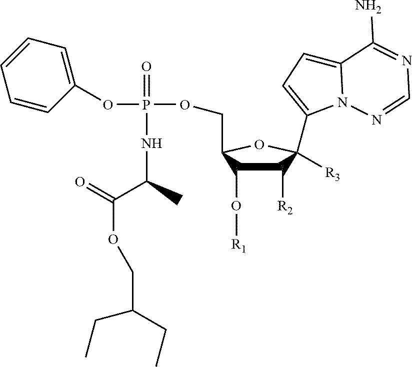

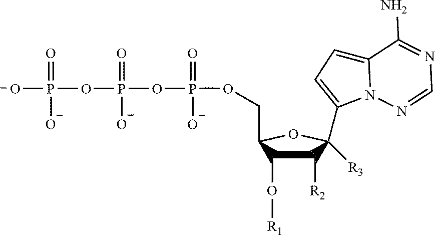

wherein R₁ is H, methyl, or small ester such as i-butyl ester and valyl ester, as long as R₁ does not prevent the recognition of the nucleotide analogue as a substrate by the viral polymerase, wherein R₂ is OH, F, H, or -O-ester such as i-butyl ester and valyl ester, wherein R₃ is F, methyl, or ethyl, and wherein the compounds depicted on the left are prodrugs of the active forms of the compounds depicted on the right.

In some embodiments, the present invention provides a composition comprising at least one of the following compounds for the treatment of viral infection caused by viruses such as SARS-CoV-2, SARS-CoV, MERS-CoV, hepatitis C virus, Marburg virus, Ebola virus and influenza virus:

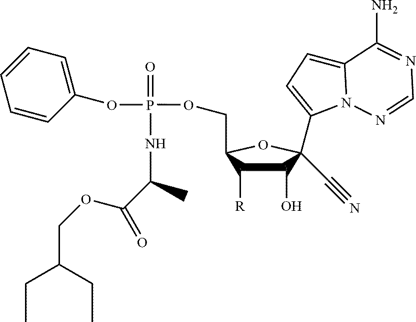

wherein R is H, F, or NH₂.

In some embodiments, the present invention provides a composition comprising at least one of the following compounds for the treatment of viral infection caused by viruses such as SARS-CoV-2, SARS-CoV, MERS-CoV, hepatitis C virus, Marburg virus, Ebola virus and influenza virus:

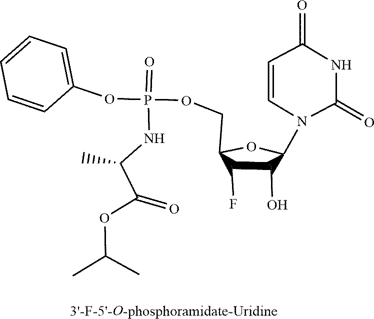

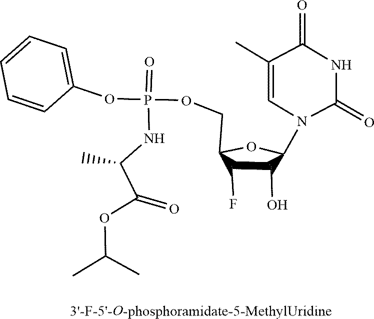

In some embodiments, the present invention provides a composition comprising at least one of the following compounds for the treatment of viral infection caused by viruses such as SARS-CoV-2, SARS-CoV, MERS-CoV, hepatitis C virus, Marburg virus, Ebola virus and influenza virus:

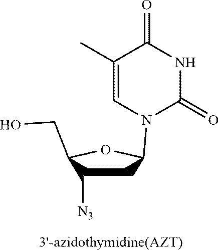

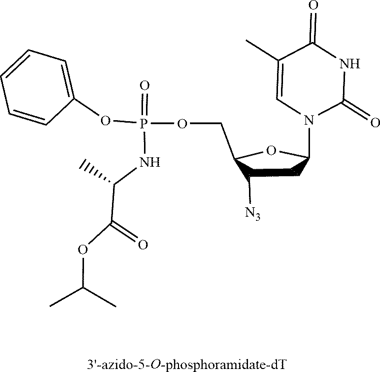

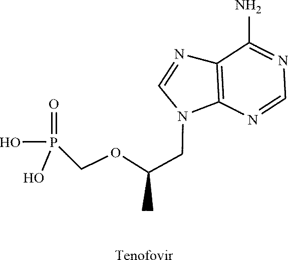

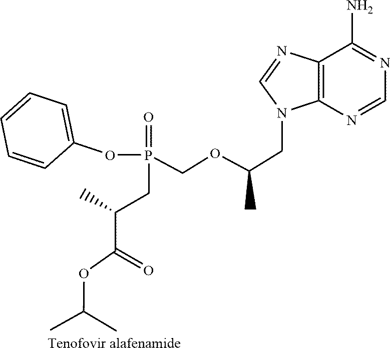

In some embodiments, the present invention provides a composition comprising at least one of the following compounds for the treatment of viral infection caused by viruses such as SARS-CoV-2, SARS-CoV, MERS-CoV, hepatitis C virus, Marburg virus, Ebola virus and influenza virus:

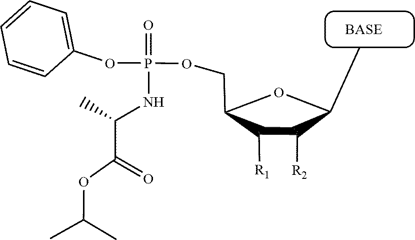

wherein BASE is A, C, G, T, U or derivatives thereof, wherein R₁ is H, methyl, F, N₃, or other small groups, as long as R₁ does not prevent the recognition of the nucleotide analogue as a substrate by the viral polymerase, wherein R₂ is H, OH, F, N₃, or -O-ester such as i-butyl ester and valyl ester, wherein R₃ is F, methyl, or ethyl,

In some embodiments, the present invention provides a composition comprising at least one of the following compounds for the treatment of viral infection caused by viruses such as SARS-CoV-2, SARS-CoV, MERS-CoV, Marburg virus, Ebola virus and influenza virus:

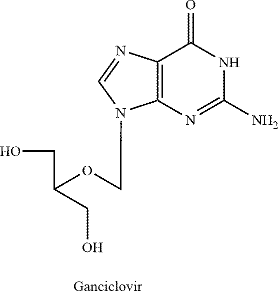

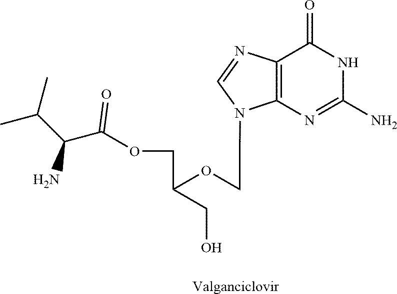

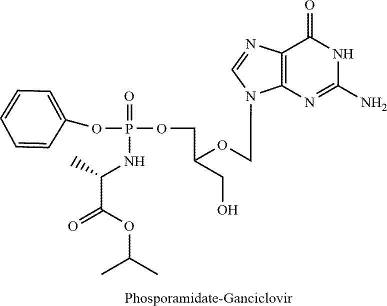

In some embodiments, the present invention provides a composition comprising at least one of the following compounds for the treatment of viral infection caused by viruses such as SARS-CoV-2, SARS-CoV, MERS-CoV, Marburg virus, Ebola virus and influenza virus:

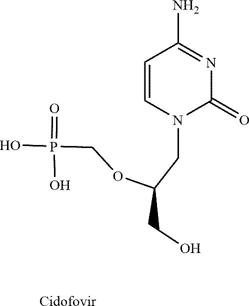

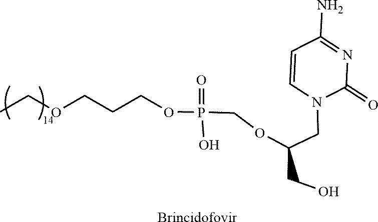

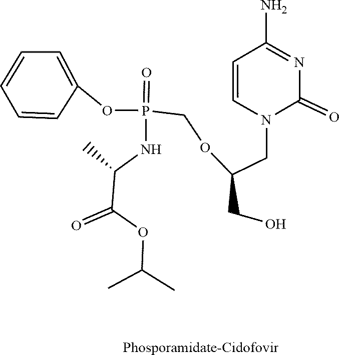

In some embodiments, the present invention provides a composition comprising at least one of the following compounds for the treatment of viral infection caused by viruses such as SARS-CoV-2, SARS-CoV, MERS-CoV, Marburg virus, Ebola virus and influenza virus:

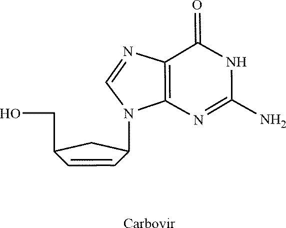

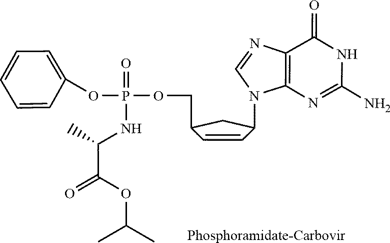

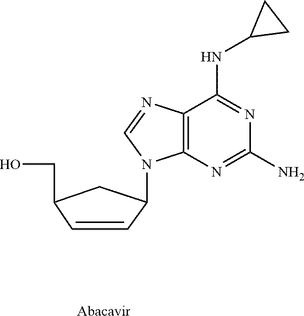

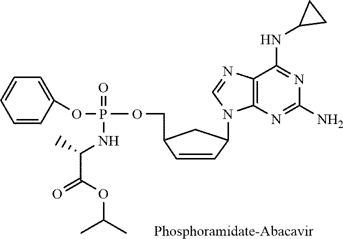

In some embodiments, the present invention provides a composition comprising at least one of the following compounds for the treatment of viral infection caused by viruses such as SARS-CoV-2, SARS-CoV, MERS-CoV, Marburg virus, Ebola virus and influenza virus:

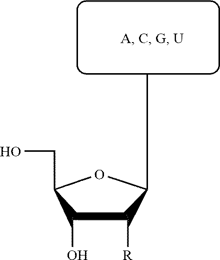

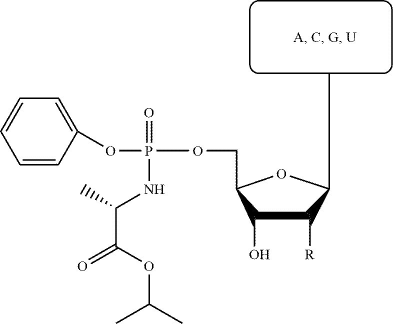

wherein R is F, OMe, NH₂, or OCH₂OCH₃.

In some embodiments, the present invention provides a composition comprising at least one of the following compounds for the treatment of viral infection caused by viruses such as SARS-CoV-2, SARS-CoV, MERS-CoV, Marburg virus, Ebola virus and influenza virus:

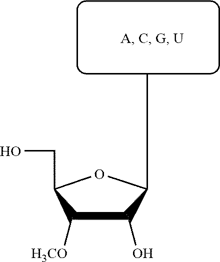

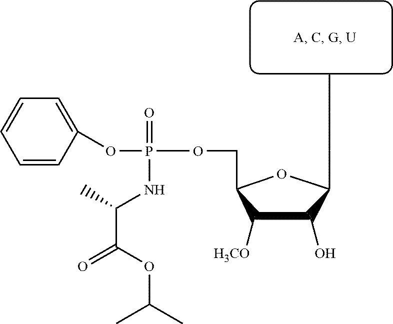

In some embodiments, the present invention provides a composition comprising at least one of the following compounds for the treatment of viral infection caused by viruses such as SARS-CoV-2, SARS-CoV, MERS-CoV, Marburg virus, Ebola virus and influenza virus:

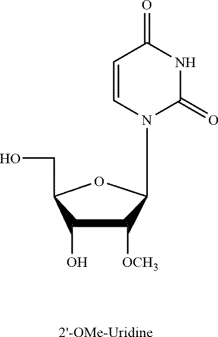

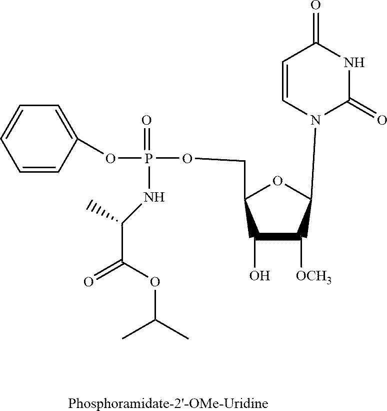

In some embodiments, the present invention provides a composition comprising at least one of the following compounds for the treatment of viral infection caused by viruses such as SARS-CoV-2, SARS-CoV, MERS-CoV, Marburg virus, Ebola virus and influenza virus:

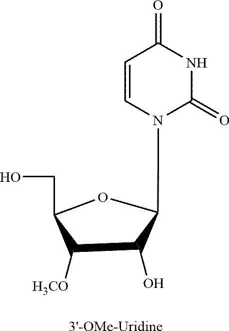

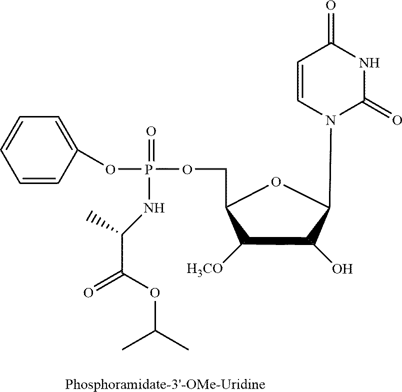

In some embodiments, the present invention provides a composition comprising at least one of the following compounds for the treatment of viral infection caused by viruses such as SARS-CoV-2, SARS-CoV, MERS-CoV, Marburg virus, Ebola virus and influenza virus:

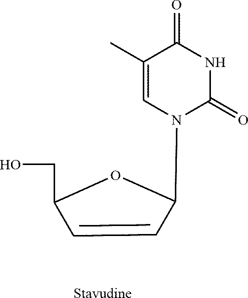

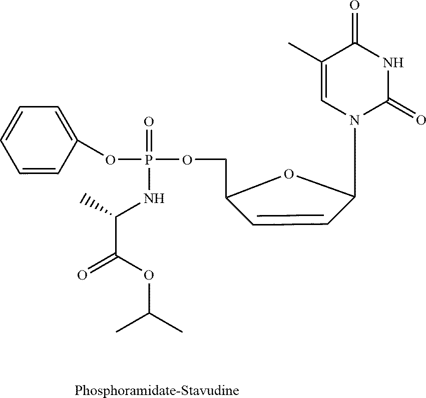

In some embodiments, the present invention provides a composition comprising at least one of the following compounds for the treatment of viral infection caused by viruses such as SARS-CoV-2, SARS-CoV, MERS-CoV, Marburg virus, Ebola virus and influenza virus:

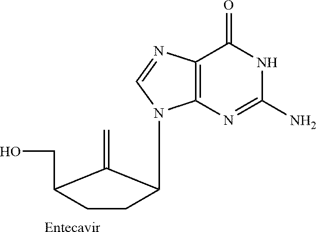

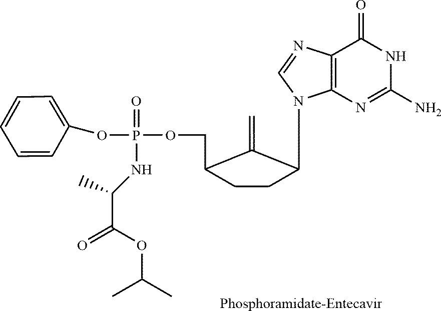

In some embodiments, the present invention provides a composition comprising at least two of the compounds disclosed herein for the treatment of viral infection caused by viruses such as SARS-CoV-2, SARS-CoV, MERS-CoV, Marburg virus, Ebola virus and influenza virus.

In some embodiments, the present invention provides a composition comprised of at least three of the compounds disclosed herein for the treatment of viral infection caused by viruses such as SARS-CoV-2, SARS-CoV, MERS-CoV, the Marburg virus, Ebola virus and influenza virus.

In some embodiments, the present invention provides a method for treating a viral infection caused by a coronavirus in a human subject afflicted with the viral infection comprising administering to the human subject a therapeutically active dose of an RdRp inhibitor such as Sofosbuvir, Remdesivir, Favipravir, Suramin and Ribavirin, an NS5A inhibitor such as Velpatasvir, Daclatasvir, Ledipasvir, Elbasvir, Ombitasvir, and Pibrentasvir, or a combination thereof, that is effective to treat the viral infection in the human subject, wherein the NS5A inhibitors inhibit the exonuclease of the coronavirus. In some preferred embodiments, the RdRp inhibitor is Sofosbuvir.

In some embodiments, the present invention provides a method for treating a viral infection caused by a coronavirus in a human subject afflicted with the viral infection comprising administering to the human subject a therapeutically active dose of an RdRp inhibitor such as Sofosbuvir, Remdesivir, Favipravir, Suramin and Ribavirin, an NS5A inhibitor such as Velpatasvir, Daclatasvir, Ledipasvir, Elbasvir, Ombitasvir, and Pibrentasvir, or a combination thereof, that is effective to treat the viral infection in the human subject, wherein the NS5A inhibitors inhibit both the exonuclease and the polymerase activities of the coronavirus. In some preferred embodiments, the RdRp inhibitor is Sofosbuvir.

In some embodiments, the present invention provides a method for treating a viral infection caused by a coronavirus in a human subject afflicted with the viral infection comprising administering to the human subject a therapeutically active dose of an RdRp inhibitor such as Sofosbuvir, Remdesivir, Favipravir, Suramin and Ribavirin, the NS5A inhibitor Velpatasvir, or a combination thereof, that is effective to treat the viral infection in the human subject, wherein Velpatasvir inhibits both the exonuclease and the polymerase activities of the coronavirus.

In some embodiments, the present invention provides a method for treating a viral infection caused by a coronavirus in a human subject afflicted with the viral infection comprising administering to the human subject a therapeutically active dose of an RdRp inhibitor such as Sofosbuvir, Remdesivir, Favipravir, Suramin and Ribavirin, the NS5A inhibitor Daclatasvir, or a combination thereof, that is effective to treat the viral infection in the human subject, wherein Daclatasvir inhibits both the exonuclease and the polymerase activities of the coronavirus.

In some embodiments, the present invention provides a method for treating a viral infection caused by a coronavirus in a human subject afflicted with the viral infection comprising administering to the human subject a therapeutically active dose of an RdRp inhibitor such as Sofosbuvir, Remdesivir, Favipravir, Suramin and Ribavirin, the NS5A inhibitor Ombitasvir, or a combination thereof, that is effective to treat the viral infection in the human subject, wherein Ombitasvir inhibits the exonuclease of the coronavirus.

In some embodiments, the present invention provides a method for treating a viral infection caused by a coronavirus in a human subject afflicted with the viral infection comprising administering to the human subject a therapeutically active dose of an RdRp inhibitor such as Sofosbuvir, Remdesivir, Favipravir, Suramin and Ribavirin, the NS5A inhibitor Pibrentasvir, or a combination thereof, that is effective to treat the viral infection in the human subject, wherein Pibrentasvir inhibits the exonuclease of the coronavirus.

In some embodiments, the present invention provides a method for treating a viral infection caused by a coronavirus in a human subject afflicted with the viral infection comprising administering to the human subject a therapeutically active dose of Sofosbuvir, Velpatasvir and Remdesivir that is effective to treat the viral infection in the human subject, wherein Velpatasvir inhibits both the exonuclease and the polymerase activities of the coronavirus.

In some embodiments, the present invention provides a method for treating a viral infection caused by a coronavirus in a human subject afflicted with the viral infection comprising administering to the human subject a therapeutically active dose of Sofosbuvir, Daclatasvir and Remdesivir that is effective to treat the viral infection in the human subject, wherein Daclatasvir inhibits both the exonuclease and the polymerase activities of the coronavirus.

A method for treating a viral infection caused by a coronavirus in a human subject afflicted with the viral infection comprising administering to the human subject a therapeutically active dose of an RdRp inhibitor such as Sofosbuvir, Remdesivir, Favipravir, Suramin and Ribavirin, an exonuclease inhibitor such as Raltegravir, Ebselen, Ritonavir and Liponavir, or a combination thereof, that is effective to treat the viral infection in the human subject.

In some embodiments, the present invention provides a method for treating a viral infection caused by a coronavirus in a human subject afflicted with the viral infection comprising administering to the human subject a therapeutically active dose of the RdRp inhibitor Sofosbuvir, an exonuclease inhibitor such as Raltegravir, Ebselen, Ritonavir and Liponavir, or a combination thereof, that is effective to treat the viral infection in the human subject.

In some embodiments, the present invention provides a method for treating a viral infection caused by a coronavirus in a human subject afflicted with the viral infection comprising administering to the human subject a therapeutically active dose of the RdRp inhibitor Sofosbuvir, an exonuclease inhibitor such as Ebselen, Ritonavir and Liponavir, or a combination thereof, that is effective to treat the viral infection in the human subject.

In some embodiments, the present invention provides a method for treating a viral infection caused by a coronavirus in a human subject afflicted with the viral infection comprising administering to the human subject a therapeutically active dose of an RdRp inhibitor such as Sofosbuvir, Remdesivir, Favipravir, Suramin and Ribavirin, the exonuclease inhibitor such as Ritonavir and Lopinavir, or a combination thereof, that is effective to treat the viral infection in the human subject.

In some embodiments, the present invention provides a method for treating a viral infection caused by a coronavirus in a human subject afflicted with the viral infection comprising administering to the human subject a therapeutically active dose of the RdRp inhibitor Sofosbuvir, an exonuclease inhibitor such as Ritonavir and Liponavir, or a combination thereof, that is effective to treat the viral infection in the human subject.

In some embodiments, the present invention provides a method for treating a viral infection caused by a coronavirus in a human subject afflicted with the viral infection comprising administering to the human subject a therapeutically active dose of an RdRp inhibitor such as Sofosbuvir, Remdesivir, Favipravir, Suramin and Ribavirin, an exonuclease inhibitor such as NS5A inhibitors, Ritonavir, Lopinavir, Ebselen and Elvitegravir, a helicase inhibitor Ranitidine bismuth citrate, or a combination thereof, that is effective to treat the viral infection in the human subject.

In some embodiments, the present invention provides a method for treating a viral infection caused by a coronavirus in a human subject afflicted with the viral infection comprising administering to the human subject a therapeutically active dose of an RdRp inhibitor such as Sofosbuvir, Remdesivir, Favipravir, Suramin and Ribavirin, a helicase inhibitor Ranitidine bismuth citrate, or a combination thereof, that is effective to treat the viral infection in the human subject.

In some embodiments, the present invention provides a method for treating a viral infection caused by a coronavirus in a human subject afflicted with the viral infection comprising administering to the human subject a therapeutically active dose of the RdRp inhibitor Sofosbuvir, the helicase inhibitor Ranitidine bismuth citrate, or a combination thereof, that is effective to treat the viral infection in the human subject.

In some embodiments, the present invention provides a method for treating a viral infection caused by a coronavirus in a human subject afflicted with the viral infection comprising administering to the human subject a therapeutically active dose of an RdRp inhibitor such as Sofosbuvir, Remdesivir, Favipravir, Suramin and Ribavirin, an NS5A inhibitor such as Velpatasvir, Daclatasvir, Ledipasvir, Elbasvir, Ombitasvir and Pibrentasvir, an NS3/4a protease inhibitor such as Grazoprevir, Voxilaprevir, Paritaprevir, Glecaprevir, Danoprevir and Telaprevir, or a combination thereof, that is effective to treat the viral infection in the human subject, wherein the NS5A inhibitors inhibit the exonuclease of the coronavirus.

In some embodiments, the present invention provides a method for treating a viral infection caused by a coronavirus in a human subject afflicted with the viral infection comprising administering to the human subject a therapeutically active dose of the RdRp inhibitor Sofosbuvir, an NS5A inhibitor such as Velpatasvir, Daclatasvir, Ledipasvir, Elbasvir, Ombitasvir and Pibrentasvir, an NS3/4a protease inhibitor comprising Grazoprevir, Voxilaprevir, Paritaprevir, Glecaprevir, Danoprevir and Telaprevir, or a combination thereof, that is effective to treat the viral infection in the human subject, wherein the NS5A inhibitors inhibit the exonuclease of the coronavirus.

In some embodiments, the present invention provides a method for treating a viral infection caused by a coronavirus in a human subject afflicted with the viral infection comprising administering to the human subject a therapeutically active dose of an RdRp inhibitor such as Sofosbuvir, Remdesivir, Favipravir, Suramin and Ribavirin, an NS5A inhibitor such as Velpatasvir, Daclatasvir, Ledipasvir, Elbasvir, Ombitasvir and Pibrentasvir, the NS3/4a protease inhibitor Voxilaprevir, or a combination thereof, that is effective to treat the viral infection in the human subject, wherein the NS5A inhibitors inhibit the exonuclease of the coronavirus.

In some embodiments, the present invention provides a method for treating a viral infection caused by a coronavirus in a human subject afflicted with the viral infection comprising administering to the human subject a therapeutically active dose the RdRp inhibitor Sofosbuvir, the NS5A inhibitor Velpatasvir, and the protease inhibitor Atazanavir, that is effective to treat the viral infection in the human subject.

In some embodiments, the present invention provides a method for treating a viral infection caused by a coronavirus in a human subject afflicted with the viral infection comprising administering to the human subject a therapeutically active dose of an RdRp inhibitor such as Sofosbuvir, Remdesivir, Favipravir, Suramin and Ribavirin, an NS5A inhibitor such as Velpatasvir, Daclatasvir, Ledipasvir, Elbasvir, Ombitasvir and Pibrentasvir, an HIV integrase inhibitor such as Elvitegravir and Raltegravir, or a combination thereof, that is effective to treat the viral infection in the human subject, wherein the NS5A inhibitor and the Elvitegravir and Raltegravir inhibit the exonuclease of the coronavirus.

In some embodiments, the present invention provides a method for treating a viral infection caused by a coronavirus in a human subject afflicted with the viral infection comprising administering to the human subject a therapeutically active dose of the RdRp inhibitor Sofosbuvir, an NS5A inhibitor such as Velpatasvir, Daclatasvir, Ledipasvir, Elbasvir, Ombitasvir and Pibrentasvir, an NS3/4a protease inhibitor such as Grazoprevir, Voxilaprevir, Paritaprevir, Glecaprevir, Danoprevir and Telaprevir, an HIV integrase inhibitor such as Elvitegravir and Raltegravir, or a combination thereof, that is effective to treat the viral infection in the human subject, wherein the NS5A inhibitor and the Elvitegravir and Raltegravir inhibit the exonuclease of the coronavirus.

In some embodiments, the present invention provides a method for treating a viral infection caused by a coronavirus in a human subject afflicted with the viral infection comprising administering to the human subject therapeutically active doses of four drugs, one each derived from four of the following classes: an RdRp inhibitor, an NS5A inhibitor, an exonuclease inhibitor, an HIV integrase inhibitor, a helicase inhibitor, and an ns3/4a protease inhibitor, wherein the NS5A inhibitor inhibits the exonuclease of the coronavirus.

In some embodiments, the present invention provides a method for treating a viral infection caused by a coronavirus in a human subject afflicted with the viral infection comprising administering to the human subject therapeutically active doses of three drugs, one each derived from three of the following classes: an RdRp inhibitor, an NS5A inhibitor, an exonuclease inhibitor, an HIV integrase inhibitor, a helicase inhibitor, and an ns3/4a protease inhibitor, wherein the NS5A inhibitor inhibits the exonuclease of the coronavirus.

In some embodiments, the present invention provides a method for treating a viral infection caused by a coronavirus in a human subject afflicted with the viral infection comprising administering to the human subject therapeutically active doses of three drugs, two derived from one of the following classes and one derived from a different one of the following classes: an RdRp inhibitor, an NS5A inhibitor, an exonuclease inhibitor, an HIV integrase inhibitor, a helicase inhibitor, and an ns3/4a protease inhibitor, wherein the NS5A inhibitor inhibits the exonuclease of the coronavirus.

In some embodiments, the present invention provides the method of the present invention, wherein the coronavirus is SARS-CoV-2 or a strain that causes SARS or MERS.

In some embodiments, the present invention provides the method of the present invention, wherein the coronavirus is SARS-CoV-2.

In some embodiments, the present invention provides a method for treating a viral infection caused by a coronavirus in a human subject afflicted with the viral infection comprising administering to the human subject therapeutically active doses of the polymerase inhibitor Sofosbuvir, the exonuclease inhibitor Ombitasvir, and a hepatitis C virus NS5A inhibitor such as Daclatasvir, Velpatasvir and Elbasvir.

In some embodiments, the present invention provides a composition for the treatment of viral infection caused by coronaviruses, hepatitis C virus, hepatitis C virus, Marburg virus, Ebola virus and influenza virus comprising one or more compounds selected from the group consisting of:

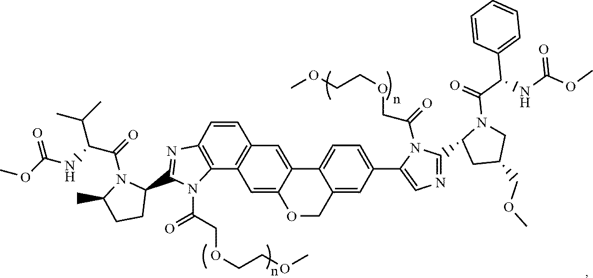

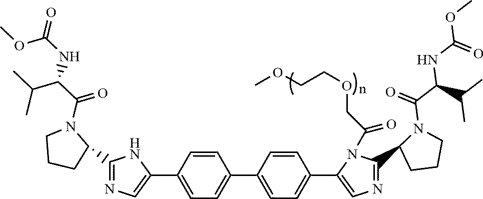

and

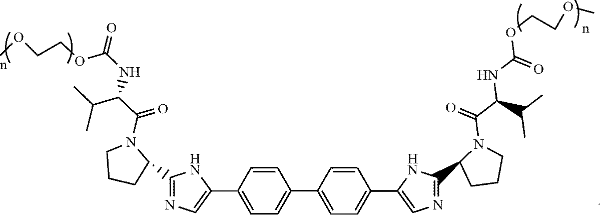

In some embodiments, the present invention provides a composition for the treatment of viral infection caused by coronaviruses, such as SARS-CoV-2 and the strains causing SARS and MERS, and/or hepatitis C virus comprising one or more compounds selected from the group consisting of:

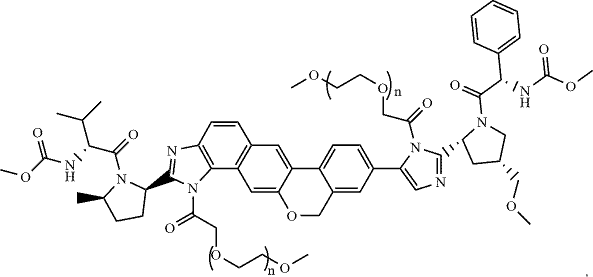

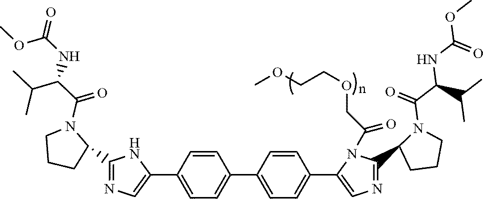

and

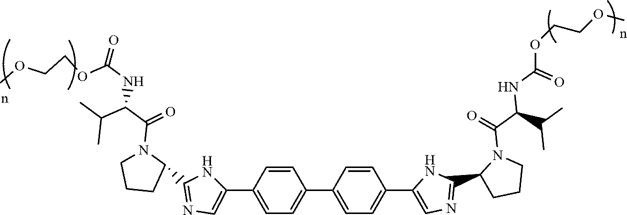

### DESCRIPTION OF FIGURES

FIG. 1: Virus-based and host-based treatment options targeting the coronavirus replication cycle. From Zumla et al 2016.

FIG. 2: The genome of SARS-CoV-2 is shown above. Proteins that cooperate to form the replication complex are shown in the middle. The use of a drug cocktail that inhibits both the RdRp (stop sign I) and the proofreading exonuclease (stop sign II) will lead to termination of the RNA polymerase reaction resulting in inhibition of RNA replication and transcription and thereby blocking viral replication.

FIG. 3: Conversion of Sofosbuvir to active drug in vivo. Adapted from Murakami et al (2010).

FIG. 4: Comparison of structures of prodrug viral inhibitors. Top: Prodrug form. Bottom: Active phosphorylated form.

FIG. 5: Incorporation of 2′-F,Me-UTP by two low fidelity polymerases but not a high fidelity polymerase. The sequence of the primer and template used for these extension reactions is shown at the top of the figure. (a-c) Incubation of the primer and template with 2′-F,Me-UTP and the appropriate buffer components for the enzymes used followed by detection of primer and extended products by MALDI-TOF MS (MS spectra for Therminator II (T2) in (a), Therminator IX (T9) in (b), and Thermo Sequenase (TS) in (c)). The detailed procedure is shown in the methods. The accuracy for m/z determination is ± 10% Da.

FIG. 6: Example structures of nucleotide analogues as viral polymerase inhibitors. These compounds all have a masked phosphate group, are unmodified or modified (R₂) at the 2′ position, and have R₁ at the 3′ position, which also comprises H.

FIG. 7: Comparison of structures of prodrug viral inhibitors. Top: Prodrug (phosphoramidate) form; Bottom: Active triphosphorylated form. Note that Sofosbuvir is an FDA approved drug for hepatitis C and 3′-N₃-dTTP (AZT) is an FDA approved drug for HIV.

FIG. 8: Synthesis of 3′-O blocked nucleoside phosphoramidate analogues. β-D-2′-deoxy-2′-α-fluoro-2′-β-C-methyl-3′-O-methyladenosine nucleoside phosphoramidate is shown as an example.

FIG. 9: Synthesis of 3′-O blocked nucleoside phosphoramidate analogues. β-D-2′-deoxy-2′-α-fluoro-3′-O-methylthiomethyluridine nucleoside phosphoramidate is shown as an example.

FIG. 10: Example synthesis of β-D-2′-deoxy-2′-α-fluoro-2′-β-C-methyl-3′-O-allyluridine nucleoside phosphoramidate.

FIG. 11A: Mode of activation of nucleotide prodrug precursors illustrated in FIG. 6 by cellular enzymes, where R₁ can also be a hydrogen.

FIG. 11B: Structure of parent prodrug 1-cyano-3′-R-substituted-4-aza-7,9-dideaza-adenosine C-nucleotide phosphoramidate (A) and its in vivo conversion to the activated triphosphate (B).

FIG. 11C: Synthesis of 1′-cyano-3′-F-s-4-aza-7,9-dideaza-adenosine C-nucleotide phosphoramidate.

FIG. 11D: Conversion of Favipiravir and its prodrug Favipiravir-ribofuranosyl-5′-O-phosphoramidate to the active form Favipiravir-ribofuranosyl-5′-O-triphosphate in vivo, a known inhibitor of the viral RNA dependent RNA polymerase.

FIG. 11E: Synthesis scheme for the prodrug Favipiravir-ribofuranosyl-5′-O-phosphoramidate.

FIG. 12: Structures of the active form of Sofosbuvir and three 3′-O modified nucleotides.

FIG. 13: A. Nucleotide analogues based on the Sofosbuvir parent structure [both the prodrug (top) and the activated triphosphate form (bottom)]. These designed nucleotide analogues can be synthesized and evaluated for their performance as inhibitors of the SARS-CoV-2 polymerase. B. Nucleotide analogues based on the Remdesivir parent structure [both the prodrug (top) and the activated triphosphate form (bottom)]. These designed nucleotide analogues can be synthesized and evaluated for their performance as inhibitors of the SARS-CoV-2 polymerase.

FIG. 14: (A) Conversion of Sofosbuvir to active 2′-F,Me-UTP drug, and (B) conversion of parent prodrug 3′-F-5′-O-phosphoramidate dT nucleoside to the activated 3′-fluoro-3′-deoxythymidine triphosphate (3′-F-dTTP) in vivo. We have evaluated their performance as inhibitors of the SARS-CoV RNA dependent RNA polymerase, as shown in FIGS. 15A and 15B.

FIG. 15A: Incorporation of 2′-F,Me-UTP, 3′-F-dTTP and 3′-N₃-dTTP by SARS-CoV RdRp to terminate the polymerase reaction. The sequence of the primer and template used for these extension reactions, which are within the N1 coding sequence of the SARS-CoV-2 genome, is shown at the top of the figure. Polymerase extension reactions were performed by incubating (a) 2′-F,Me-UTP, (b) 3′-F-dTTP, and (c) 3′-N₃-dTTP with pre-assembled SARS-CoV polymerase (nsp12, nsp7 and nsp8), the indicated RNA template and primer, and the appropriate reaction buffer, followed by detection of reaction products by MALDI-TOF MS. The detailed procedure is shown in the Methods section. For comparison, data for extension with UTP are presented in FIG. 15B. The accuracy for m/z determination is ± 10 Da.

FIG. 15B: Incorporation of UTP by SARS-CoV RNA-dependent RNA polymerase. The details of the method are similar to FIG. 15A and further details are provided in the text.

FIG. 16: Conversion of parent prodrug 3′-N₃-5′-O-phosphoramidate dT nucleoside (top left) or azidothymidine (AZT, top right) to the activated 3′-N₃-3′-deoxythymidine triphosphate (3′-N₃-dTTP) in vivo. We have evaluated the performance of the 3′-N₃-dTTP as an inhibitor of the SARS-CoV RNA dependent RNA polymerase, as shown in FIG. 15A.

FIG. 17: A. Nucleotide analogues based on 3′-F-Uridine parent structure [both the prodrug (3′-F-5′-O-phosphoramidate-Uridine top) and the activated triphosphate form (3′-F-UTP, bottom)]. B. Nucleotide analogues based on the 3′-F-5-MethylUridine parent structure [both the prodrug (3′-F-5′-O-phosphoramidate-5-MethylUridine, top) and the activated triphosphate form (3′-F-5-Me-UTP, bottom)]. These designed nucleotide analogues can be synthesized and evaluated for their performance as inhibitors of the SARS-CoV-2 polymerase.

FIG. 18: Structures of prodrug viral inhibitors. Prodrugs Tenofovir alafenamide (TAF) (a), Emtricitabine-5′-O-phosphoramidate (d) and Emtricitabine (e), their monophosphate forms Tenofovir (TFV) and Emtricitabine monophosphate (b and f, respectively), and their active triphosphate forms (c and g, respectively).

FIG. 19: Structures of Emtricitabine derivatives. Left: Prodrug (phosphoramidate) form; Right: Active triphosphate form.

FIG. 20: Structures of 3 viral inhibitors. Top: Nucleoside form; Bottom: Active triphosphate form.

FIG. 21: Incorporation of 2′-F,Me-UTP, 3′-F-dTTP, TFV-DP and 3′-N₃-dTTP by SARS-CoV-2 RdRp to terminate the polymerase reaction. The sequences of the primer and template used for these extension reactions, which are at the 3′ end of the SARS-CoV-2 genome, are shown at the top of the figure. Polymerase extension reactions were performed by incubating (a) 2′-F,Me-UTP, (b) 3′-F-dTTP, (c) UTP + TFV-DP, and (d) 3′-N₃-dTTP with pre-assembled SARS-CoV-2 polymerase (nsp12, nsp7 and nsp8), the indicated RNA template and primer, and the appropriate reaction buffer, followed by detection of reaction products by MALDI-TOF MS. The accuracy for m/z determination is ± 10 Da. Further details are in the text.

FIG. 22: Incorporation of TFV-DP and Car-TP by SARS-CoV-2 RdRp to terminate the polymerase reaction. The details of the method are similar to those indicated in FIG. 21 and further details are in the text.

FIG. 23: Incorporation of Lam-TP and Ec-TP by SARS-CoV-2 RdRp catalyzed reaction. The details of the method are similar to those indicated in FIG. 21 and further details are in the text.

FIG. 24: Example chemical structures of modified nucleoside triphosphates used for evaluation of SARS-CoV-2 polymerase reaction inhibition.

FIG. 25: Structures of viral nucleoside inhibitors, possible prodrugs and active triphosphate forms. The nucleosides 2′-O-Me-uridine, 2′-F-uridine and 3′-O-Me-uridine (left), example prodrug forms (middle) and their active triphosphate forms (right).

FIG. 26: Structures of viral nucleoside inhibitors, example prodrugs and active triphosphate forms. The compounds Ganciclovir, Cidofovir, Carbovir, Stavudine and Entecavir (left), example prodrug forms (middle) and their active triphosphate forms (right).

FIG. 27: Incorporation of 2′-O-Me-UTP, Sta-TP and Biotin-dUTP by SARS-CoV-2 RdRp to terminate the polymerase reaction. The sequences of the primer and template used for this extension reaction, which are at the 3′ end of the SARS-CoV-2 genome, are shown at the top of the figure. Polymerase extension reactions were performed by incubating 2′-O-Me-UTP (a), Sta-TP (b) and Biotin-dUTP (c) with pre-assembled SARS-CoV-2 polymerase (nsp12, nsp7 and nsp8), the indicated RNA template and primer, and the appropriate reaction buffer, followed by detection of reaction products by MALDI-TOF MS. The detailed procedure is shown in the text. The accuracy for m/z determination is ± 10 Da.

FIG. 28: Incorporation of Cid-DP by SARS-CoV-2 RdRp to achieve delayed termination of the polymerase reaction. The details of the method are similar to those in FIG. 27 and further details are in the text.

FIG. 29: Incorporation of Car-TP, Ent-TP and Gan-TP by SARS-CoV-2 RdRp to terminate the polymerase reaction. The details of the method are similar to those in FIG. 27 and further details are in the text.

FIG. 30: Incorporation of 2′-O-Me-UTP and 3′-O-Me-UTP by SARS-CoV RdRp to terminate the polymerase reaction. The details of the method are similar to those in FIG. 27 and further details are in the text.

FIG. 31: Incorporation of 2′-F-dUTP by SARS-CoV RdRp to terminate the polymerase reaction. The details of the method are similar to those in FIG. 27 and further details are in the text.

FIG. 32: Incorporation of Desthiobiotin-UTP (Desthio-UTP) by SARS-CoV-2 RdRp. The details of the method are similar to those in FIG. 27 and further details are in the text.

FIG. 33: Incorporation of 2′-O-Me-UTP and dUTP by SARS-CoV-2 RdRp. The details of the method are similar to those in FIG. 27 and further details are in the text.

FIG. 34: Incorporation of Cid-DP by SARS-CoV RdRp to terminate the polymerase reaction. The details of the method are similar to those in FIG. 27 and further details are in the text.

FIG. 35: Incorporation of Car-TP and Gan-TP by SARS-CoV RdRp to terminate the polymerase reaction. The details of the method are similar to those in FIG. 27 and further details are in the text.

FIG. 36: Comparison of incorporation efficiencies of UTP, dUTP, Biotin-dUTP, 2′-F-dUTP, 2′-O-Me-UTP and 2′-NH₂-dUTP by SARS-CoV-2 RdRp. The details of the method are similar to those in FIG. 27 and further details are in the text.

FIG. 37: Incorporation of Sta-TP and Cid-DP by SARS-CoV-2 RdRp to terminate the polymerase reaction. The details of the method are similar to those in FIG. 27 and further details are in the text.

FIG. 38. SARS-CoV-2 exonuclease activity with a cytosine terminated RNA template-loop-primer. A mixture of 500 nM of template-loop-primer H4-C containing a 3′ terminal cytosine (shown at the top of the figure), 250 nM nsp14 and 1 µM nsp10 was incubated in a buffer solution (40 mM Tris pH 8, 5 mM DTT, 1.5 mM MgCl₂, 50 µM ZnCl₂) at 37° C. for 5 minutes (b) or 30 minutes (c). The same protocol was carried out in the absence of nsp14/nsp10 (a). Products of the exonuclease reaction were detected by MALDI-TOF MS. The signal intensity was normalized to the highest peak. The accuracy for m/z determination is ± 10 Da.

FIG. 39: SARS-CoV-2 exonuclease activity with a 2′-methoxycytosine terminated RNA template-loop-primer. The details of the method are similar to those in FIG. 38 and further details are in the text.

FIG. 40: SARS-CoV-2 exonuclease activity with a 2′-fluoro-2′-deoxycytosine terminated RNA template-loop-primer. The details of the method are similar to those in FIG. 38 and further details are in the text.

FIG. 41: SARS-CoV-2 exonuclease activity with a deoxycytosine terminated RNA template-loop-primer. The details of the method are similar to those in FIG. 38 and further details are in the text.

FIGS. 42A and 42B: Treatment of the RNA products with exonuclease to determine relative excision of UMP (a-d), Biotin-dUMP (e-h), Stavudine-MP (i-1) and Carbovir-MP (m-p). Untreated products (0 min) are shown in (a) for UMP extended RNA, (e) for Biotin-dUMP extended RNA, (i) for Stavudine-MP extended RNA and (m) for Carbovir-MP. Exonuclease reactions were performed by incubating the purified RNA products, either synthetic (UMP) generated using the same procedure as in FIGS. 38-41 or by extension with reverse transcriptase (Biotin-dUTP, Stavudine-TP, Carbovir-TP), with preassembled SARS-CoV-2 exonuclease complex (nsp14 and nsp10) for 5 min (b, f, j, n), 15 min (c, g, k, o) and 30 min (d, h, 1, p), followed by detection of reaction products by MALDI-TOF MS. The signal intensities were normalized to the highest peak within each time series. The accuracy for m/z determination is approximately ± 10 Da.

FIG. 43: Example structures of C5-modified pyrimidine and C7-modified purine nucleotide phosphoramidate prodrugs.

FIG. 44: A, In vivo conversion of β-D-2′-deoxy-2′-α-fluoro-2′-β-C-methyl-C5-substituted-uridine nucleotide phosphoramidate to activated triphosphate, and B, conversion of parent prodrug 1′-cyano-4-aza-7,9-dideaza-C7-substituted-adenosine C-nucleotide phosphoramidate to the activated triphosphate.

FIG. 45: Synthesis of C5-substitute-nucleotide phosphoramidate analogues (Sofosbuvir analogue as example) : β-D-2′-deoxy-2′-α-fluoro-2′-β-C-methyl-C5-substituted-uridine nucleotide phosphoramidate.

FIG. 46: Synthesis of C7-substituted-nucleotide phosphoramidate analogues (Remdesivir analogue as example): 1′-cyano-4-aza-7,9-dideaza-C7-substituted-adenosine C-nucleotide phosphoramidate.

FIG. 47: Polymerase reactions with SOF-TP, UTP or UTP + RDV-TP to produce RNA extension products in preparation for exonuclease reactions in FIGS. 48-49. The sequence of the RNA template-loop-primer used for these polymerase extension reactions is shown at the top of the figure. (a) MALDI-TOF mass spectrum of the unextended RNA. Polymerase extension reactions were performed by incubating (b) SOF-TP, (c) UTP and (d) UTP + RDV-TP with pre-assembled SARS-CoV-2 polymerase (nsp12, nsp7 and nsp8) and the indicated RNA template-loop-primer, followed by detection of reaction products by MALDI-TOF MS. The accuracy for m/z determination is approximately ± 10 Da.

FIG. 48: Treatment of the RNA products from FIG. 47 with exonuclease and analysis by MALDI-TOF MS to determine relative excision of Sofosbuvir, UMP and Remdesivir. Untreated products (0 min) are shown in (a) for SOF extended RNA, (d) for UMP extended RNA and (g) for UMP plus RDV extended RNA. Exonuclease reactions were performed by incubating the purified RNA products, generated using the same procedure as in FIG. 47, with preassembled SARS-CoV-2 exonuclease complex (nsp14 and nsp10) for 5 min (b,e,h) or 30 min (c,f,i), followed by detection of reaction products by MALDI-TOF MS. The signal intensities were normalized to the highest peak within each time series. The accuracy for m/z determination is approximately ± 10 Da.

FIG. 49: Treatment of the RNA products from FIG. 47 with exonuclease and then combined for analysis by MALDI-TOF MS to determine relative excision of Sofosbuvir, UMP and Remdesivir. Details are similar to those in FIG. 48 and are described in the text.

FIG. 50: Treatment of the unextended RNA template-loop-primer (sequence shown at the top of the figure) with exonuclease as a control and analyzed by MALDI-TOF MS. The untreated RNA (0 min) is shown in (a). Exonuclease reactions were performed by incubating the RNA with the preassembled SARS-CoV-2 exonuclease complex (nsp14 and nsp10) for 5 min (b) or 30 min (c), followed by detection of reaction products by MALDI-TOF MS. Other details are similar to those in FIG. 48 and described in the text.

FIG. 51A: HCV NS5A inhibitors: Daclatasvir, Velpatasvir, Ledipasvir, Elbasvir, Ombitasvir and Pibrentasvir.

FIG. 51B: Ethylene glycol moieties attached to Velpatasvir.

FIG. 51C: Ethylene glycol moieties attached to Daclatasvir.

FIG. 52: Inhibition by Daclatasvir of SARS-CoV-2 RdRp complex catalyzed U extension. A mixture of 500 nM RNA template-loop-primer (shown at the top of the figure), 1 µM SARS-CoV-2 pre-assembled RdRp complex (nsp12/nsp7/nsp8) and 3 µM UTP was incubated in buffer solution at 30° C. for 1 hour in the absence (B) or presence of daclatasvir at 1 µM (C), 4 µM (D), 16 µM (E) and 64 µM (F). The RNA template-loop-primer (A) and the products of the polymerase extension reaction (B-F) were analyzed by MALDI-TOF MS. The signal intensity was normalized to the highest peak. The accuracy for m/z determination is ± 10 Da. Reaction conditions were selected to yield an incorporation efficiency of approximately 70% as seen by MALDI-TOF-MS analysis in B. The peak at 7851 Da corresponds to the RNA template-loop-primer (7851 Da expected) and the peak at 8156 Da corresponds to the U extended RNA product (8157 Da expected). Addition of Daclatasvir reduced the amount of the U extended RNA product in a concentration-dependent manner. A plot for the inhibition of the polymerase reaction vs. the Daclatasvir concentration is shown in G. Additional details are provided in the text.

FIG. 53: Inhibition by Daclatasvir of SARS-CoV-2 RdRp complex catalyzed Sofosbuvir extension. Details are similar to those of FIG. 52 and are described in the text. A plot for the inhibition of the polymerase reaction to incorporate SFV-TP into RNA vs. the daclatasvir concentration is shown in G; for comparison, the plot for incorporation of U into RNA from FIG. 52 is also included in G.

FIG. 54: Inhibition by Velpatasvir of SARS-CoV-2 RdRp complex catalyzed U extension. Details are similar to those of FIG. 52 and are described in the text.

FIG. 55: Inhibition of SARS-CoV-2 exonuclease by Daclatasvir and Velpatasvir.

FIG. 56: Inhibition of SARS-CoV-2 exonuclease by Ritonavir.

FIG. 57: Inhibition of SARS-CoV-2 exonuclease by Lopinavir.

FIG. 58: Inhibition of SARS-CoV-2 exonuclease by Ebselen.

FIG. 59: Inhibition of SARS-CoV-2 exonuclease by Ombitasvir, Elvitegravir, Ledipasvir, Elbasvir and Pibrentasvir.

FIG. 60: Inhibition of SARS-CoV-2 exonuclease by Ombitasvir and Pibrentasvir.

FIG. 61: HCV NS3-4 A inhibitors: Paritaprevir, Glecaprevir, Voxilaprevir, Grazoprevir, Danoprevir and Telaprevir.

FIG. 62: HIV Integrase inhibitors: Bictegravir, Dolutegravir, Elvitegravir and Raltegravir.

FIG. 63: Protease inhibitors: Atazanavir, Ritonavir and Lopinavir.

FIG. 64: Exonuclease inhibitor: Ebselen.

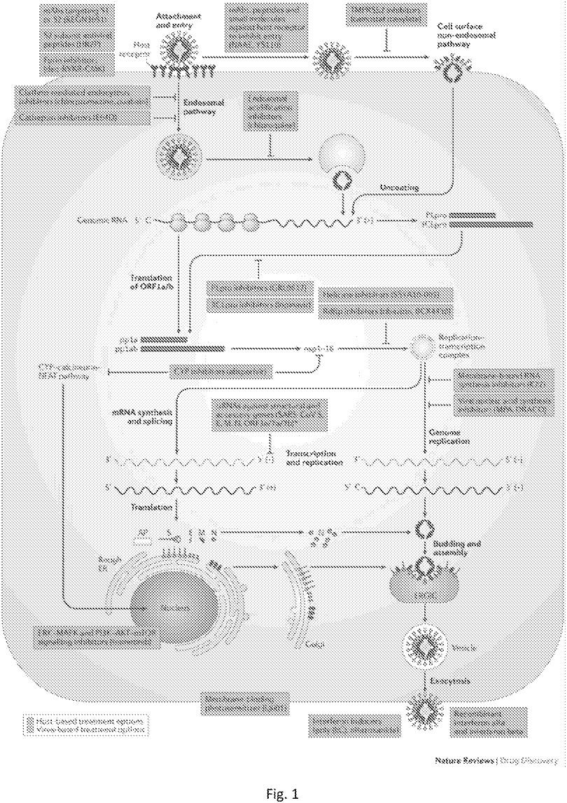

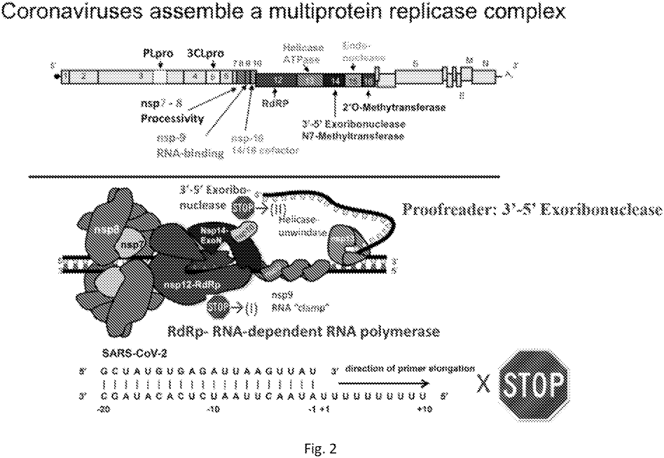

### DETAILED DESCRIPTION OF THE INVENTION

### Nucleoside Triphosphates as Inhibitors of the Coronavirus RdRps

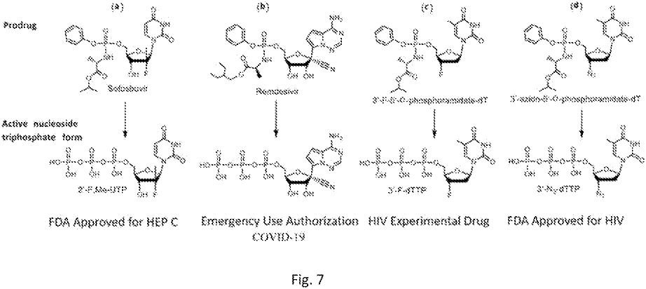

Based on our similar insight related to their molecular structures and previous antiviral activity studies, in comparison with Sofosbuvir, we selected the triphosphate forms of Alovudine (3′-deoxy-3′-fluorothymidine, FIG. 7c) and azidothymidine (AZT, the first FDA approved drug for HIV/AIDS, FIG. 7d) for evaluation as inhibitors of the SARS-CoV RdRp. These two compounds share a similar backbone structure (base and ribose) with Sofosbuvir, but have only one modification group at the 3′ carbon of the deoxyribose. Furthermore, because these modifications on Alovudine and AZT are on the 3′ carbon in place of the OH group, they directly prevent further incorporation of nucleotides leading to termination of RNA synthesis and replication of the virus if the exonuclease activity is also inhibited and if these two compounds can compete with their natural counterparts. Both Alovudine and AZT are deoxythymidine analogues. However, because their size, structure and base-pairing properties are similar to uridine and the SARS-CoV RdRp has low fidelity, the triphosphate forms of these two analogues might still be substrates of the viral polymerase.

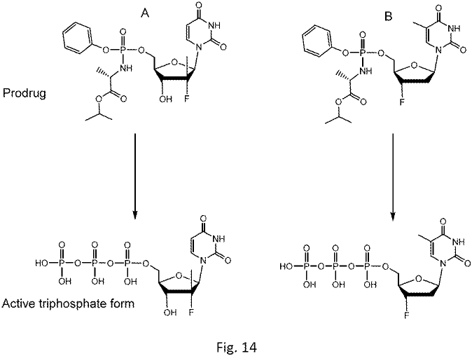

Alovudine is one of the most potent inhibitors of HIV reverse transcriptase and HIV-1 replication (Camerman et al 1990). AZT is another antiretroviral medication which has long been used to prevent and treat AIDS (Mitsuya et al 1985, Yarchoan et al 1986, Mitsuya et al 1990). Upon entry into the infected cells, similar to Alovudine (FIG. 14), cellular enzymes convert AZT into the effective 5′-triphosphate form (3′-N₃-dTTP, structure shown in FIG. 7), which competes with dTTP for incorporation into DNA by HIV-reverse transcriptase resulting in termination of HIV’s DNA synthesis (Furman et al 1986).

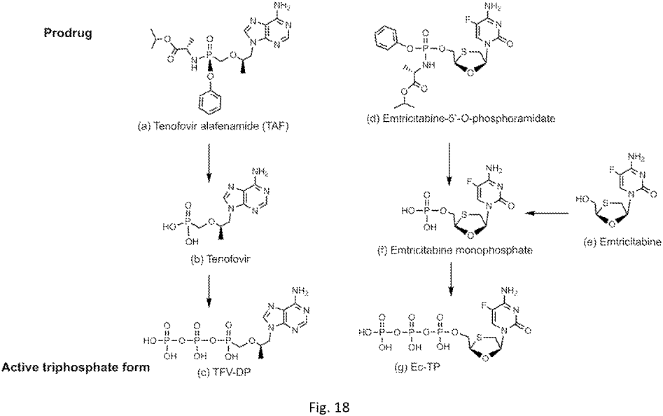

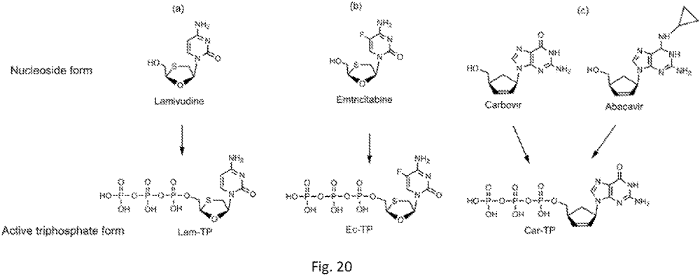

Using similar structure-activity based molecular insight, we selected the active triphosphate form of Tenofovir alafenamide (TAF, Vemlidy, an acyclic adenosine nucleotide) (FIG. 18c), which is an FDA approved drug for the treatment of HIV and hepatitis B virus (HBV) infection, for evaluation as a SARS-CoV-2 RdRp inhibitor. Similarly, we also selected the triphosphates of three HIV RT inhibitors, Lamivudine triphosphate (Lam-TP, FIG. 20a), Emtricitabine triphosphate (Ec-TP, FIGS. 18g, 20b) and Carbovir triphosphate (Car-TP, FIG. 20c) to test their ability to inhibit the SARS-CoV-2.

TAF, a prodrug form of the nucleotide analogue viral polymerase inhibitor Tenofovir (TFV), shows potent activity for HIV and HBV, but only limited inhibition of host nuclear and mitochondrial polymerases (Lou 2013, De Clercq 2016). It is activated by a series of hydrolases to the deprotected monophosphate form, TFV, and then by two consecutive kinase reactions to the triphosphate form Tenofovir diphosphate (TFV-DP) (Birkus et al 2016) (FIGS. 18a-c) . TFV-DP is an acyclic nucleotide and does not have a 3′-OH group. It is incorporated by both HIV and HBV polymerases, terminating nucleic acid elongation and viral replication (Lou 2013, Birkus et al 2016). As a noncyclic nucleotide, TFV-DP lacks a normal sugar ring configuration, and thus it is less likely to be recognized by 3′-exonucleases involved in SARS-CoV-2 proofreading processes, decreasing its likelihood of developing resistance to the exonuclease (Smith et al 2013) .

The oral drug Lamivudine (3TC) is a cytidine analogue containing an oxathiolane ring with an unnatural (-)-β-L-stereochemical configuration, making it a poor substrate for host DNA polymerases (Quercia et al 2018). This prodrug, which can be taken orally and has low toxicity, is converted by cellular enzymes, first to a monophosphate, then to the active triphosphate form, Lam-TP. Emtricitabine (Emtriva, FTC) has a similar structure to Lamivudine but with a fluorine at the 5-position of the cytosine (Hung et al 2019). Conversion of the prodrug form to the active triphosphate (FIGS. 18e-g) is analogous to the activation mechanism for Lamivudine. Like TAF, 3TC and FTC are effective against HBV (Lim et al 2006) . The absence of an OH group at the 3′ position of both Lam-TP and Ec-TP ensures that once these nucleotide analogues are incorporated into the primer in the polymerase reaction, no further incorporation of nucleotides by the polymerase can occur. Car-TP is a carbocyclic guanosine didehydro-dideoxynucleotide. The parent prodrug, Abacavir (Ziagen), is an FDA-approved nucleoside RT inhibitor used for HIV/AIDS treatment (Faletto et al 1997, Ray et al 2002).

In addition to the above nucleotide analogues, we identified additional nucleotide analogues with a larger variety of modifications for evaluation of efficient termination of the polymerase reaction; we also considered the chemical or structural properties of these compounds that may help them overcome the virus′ proofreading function. These nucleotide analogues were selected based on one or more of the following criteria. First, they have structural and chemical properties such as (a) similarity in size and structure to natural nucleotides, including the ability to fit within the active site of the polymerase, (b) presence of a small 3′-OH capping group or absence of a 3′-OH group resulting in obligate termination of the polymerase reaction; or (c) modifications at the 2′ or other positions on the sugar or base that can potentially lead to termination. We previously showed that nucleotides with substantial modifications on the base can be incorporated by DNA polymerases (Ju et al. 2006). The criteria above provide structural and chemical features that we can explore allowing them to evade viral exonuclease activity (Minskaia et al. 2006). Second, if they have previously been shown to inhibit the polymerases of other viruses, even those with different polymerase types, they may have the potential to inhibit the SARS-CoV-2 RdRp, as we have shown for HIV reverse transcriptase (RT) inhibitors (Ju et al. 2020a,b; Chien et al. 2020a,b; Jockusch et al. 2020a,b). Third, ideally, the inhibitors should display high selectivity for viral polymerases relative to cellular DNA or RNA polymerases. Fourth, there is an advantage in considering nucleotide analogues that are the active triphosphate forms of FDA-approved drugs, as these drugs are known to have acceptable levels of toxicity and are more likely to be tolerated by patients with coronavirus infections, including COVID-19.

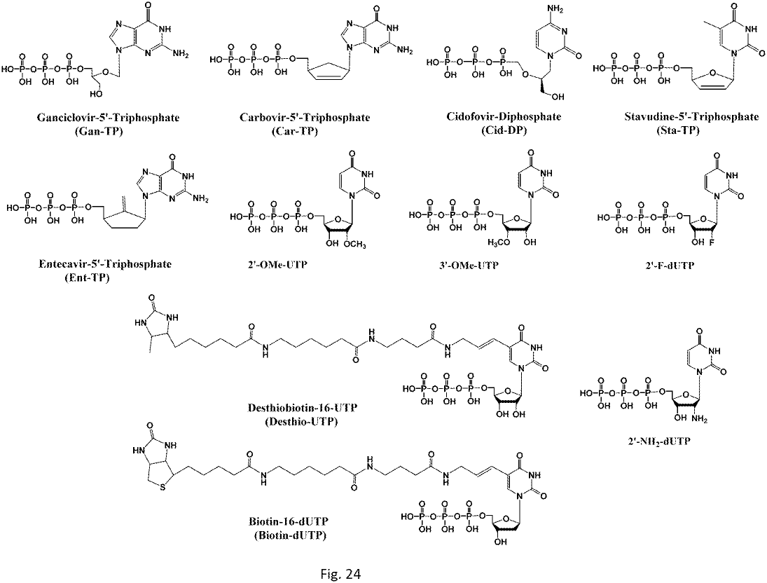

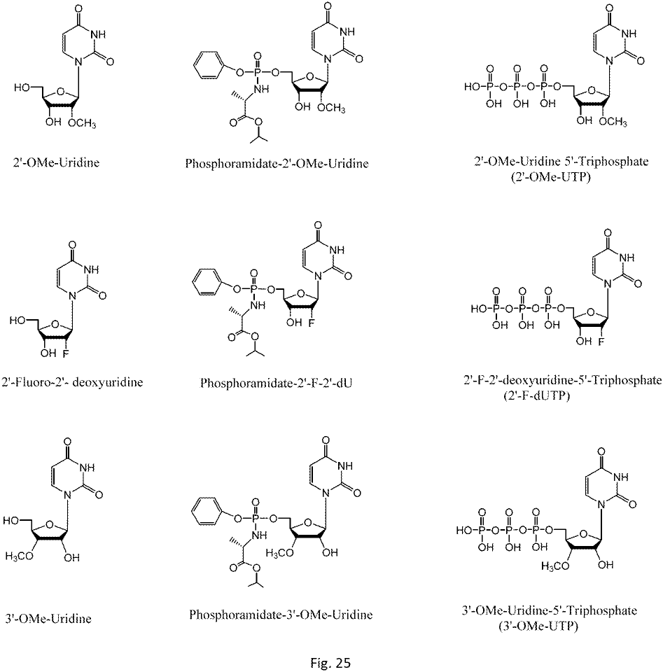

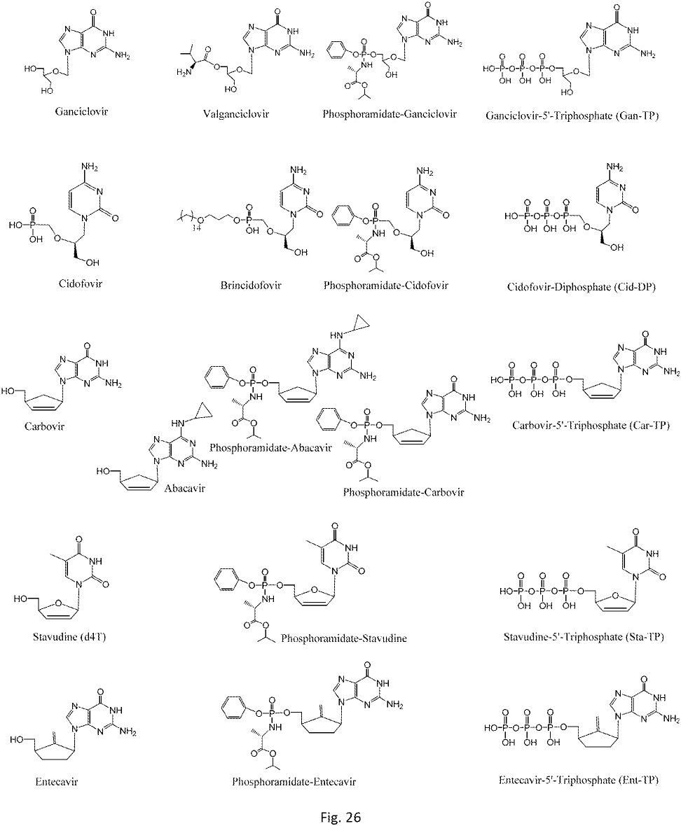

The following 11 nucleotide analogues with sugar or base modifications (structures shown in FIG. 24) were selected for evaluation of their ability to inhibit the SARS-CoV-2 or SARS-CoV RdRps: Ganciclovir 5′-triphosphate, Carbovir 5′-triphosphate, Cidofovir diphosphate, Stavudine 5′-triphosphate, Entecavir 5′-triphosphate, 2′-O-methyluridine-5′-triphosphate (2′-OMe-UTP), 3′-0-methyluridine-5′-triphosphate (3′-OMe-UTP), 2′-fluoro-2′-deoxyuridine-5′-triphosphate (2′-F-dUTP), desthiobiotin-16-aminoallyl-uridine-5′-triphosphate (Desthiobiotin-16-UTP), biotin-16-aminoallyl-2′-deoxyuridine-5′-triphosphate (Biotin-16-dUTP) and 2′-amino-2′-deoxyuridine-5′-triphosphate (2′-NH₂-dUTP). The nucleoside and prodrug forms for the FDA-approved drugs are shown in FIG. 26; nucleoside and potential prodrug forms for three other nucleotide analogues are shown in FIG. 25.

Some of the uridine analogues listed above have been previously shown to be substrates of viral polymerases (Arup et al. 1992; Lauridsen et al. 2012). The 2′-O-methyluridine triphosphate is of particular interest since 2′-O-methyl nucleotides can resist removal by the 3′-exonuclease found in coronaviruses (Minskaia et al. 2006). We describe the properties of the 5 nucleotide analogues whose prodrug forms are FDA-approved for other virus infections as follows.

Ganciclovir triphosphate (Gan-TP) is an acyclic guanosine nucleotide (FIG. 24) . The parent nucleoside Ganciclovir (Cytovene, FIG. 26) is used to treat AIDS-related cytomegalovirus (CMV) infections. The drug can inhibit herpesviruses and varicella zoster virus. The valyl ester prodrug Valganciclovir (FIG. 26) can be given orally. After cleavage of the valyl ester, Ganciclovir is converted to Ganciclovir triphosphate by viral and cellular enzymes to inhibit the viral polymerase (Matthews and Boehme 1988; Akyürek et al. 2001). Carbovir triphosphate (Car-TP) is a carbocyclic guanosine didehydro-dideoxynucleotide (FIG. 24). The parent prodrug, Abacavir (Ziagen, FIG. 26), is an FDA-approved nucleoside RT inhibitor for HIV/AIDS treatment (Faletto et al. 1997; Ray et al. 2002). It is taken orally and is well tolerated.

Cidofovir diphosphate (Cid-DP) is an acyclic cytidine nucleotide (FIG. 24). Its prodrug form Cidofovir (Vistide, FIG. 26) is an FDA-approved intravenous drug for the treatment of AIDS-related CMV retinitis and has been used off-label for a variety of DNA virus infections (De Clercq 2002; Lanier et al. 2010). A second prodrug form of Cidofovir diphosphate, Brincidofovir (FIG. 26), is an oral antiviral drug with a lipid moiety masking the phosphate group and a candidate for treating smallpox infections. It is active against a wide range of DNA viruses in animals (Trost et al. 2015; Cundy et al. 1999). Both Brincidofovir and a ProTide-based prodrug (Table 1) are expected to enter cells rapidly. Although Cidofovir is incorporated into DNA in the polymerase reaction by vaccinia virus DNA polymerase, the termination of synthesis occurs after extension by an additional nucleotide, a delayed termination similar to that shown for Remdesivir for coronavirus RdRp; Cidofovir incorporated in the penultimate position of the DNA extension strand by the vaccinia virus polymerase is not removed by the viral 3′-exonuclease (Magee et al. 2005). Stavudine triphosphate (FIG. 24), a thymidine analogue, is the active triphosphate form of Stavudine (d4T, Zerit, FIG. 26), an antiviral used for the prevention and treatment of HIV/AIDS (Ho and Hitchcock 1989) via inhibition of the HIV RT (Huang et al. 1992). The lack of a 3′-OH group makes it an obligate inhibitor. Entecavir triphosphate (Ent-TP, FIG. 24), the active triphosphate form of the oral drug Entecavir (Baraclude, FIG. 26), is a guanosine nucleotide inhibitor of the hepatitis B virus polymerase (Matthews 2006, Rivkina & Rybalow 2002). It shows little if any inhibition of nuclear and mitochondrial DNA polymerases (Mazzucco et al. 2008) and has generally been shown to have low toxicity. Entecavir triphosphate is a delayed chain terminator of the HIV-1 reverse transcriptase, making it resistant to phosphorolytic excision (Tchesnokov et al. 2008).

Once these nucleotide analogues are incorporated into a RNA primer in the polymerase reaction, the fact that they lack either a normal sugar ring configuration or the 2′- and/or 3′-OH groups would make them less likely to be removed by the 3′-exonuclease involved in SARS-CoV-2 proofreading.

### Coronaviruses Have a Proofreading Exonuclease Activity That Must Be Overcome to Develop Effective SARS-CoV-2 RdRp Nucleotide Inhibitors

In contrast to many other RNA viruses, SARS-CoV and SARS-CoV-2 have very large genomes that encode a 3′-5′ exonuclease (nsp14) involved in proofreading (Ma et al. 2015; Shannon et al. 2020), the activity of which is enhanced by the cofactor nsp10 (Bouvet et al. 2012). This proofreading function increases replication fidelity by removing mismatched nucleotides (Ferron et al. 2018). Mutations in nsp14 led to reduced replication fidelity of the viral genome (Eckerle et al. 2010). Interestingly, while the nsp14/nsp10 complex efficiently excises single mismatched nucleotides at the 3′ end of the RNA chain, it is not able to remove longer stretches of unpaired nucleotides or 3′ modified RNA (Bouvet et al. 2012). For the nucleotide analogues to be successful inhibitors of the RdRps of these viruses, they need to overcome this proofreading function. It was reported that the coronavirus exonuclease activity typically requires a 2′-OH group at the 3′ end of the growing RNA strand (Minskaia et al. 2006). However, in instances of delayed termination in which the offending nucleotide analogue is no longer at the 3′ end, they will not be removed by the exonuclease (Bouvet et al. 2012; Gordon et al. 2020a; 2020b). Nearly all the nucleotide analogues described above lack the 2′-OH group, have modifications that block the 2′-OH group on the sugar, or are acyclic nucleotide derivatives. Such nucleotides are less likely to be substrates of viral exonucleases.

We first described the use of Sofosbuvir as a possible treatment for COVID-19 in January 2020 (Ju et al 2020a); since then, additional studies have appeared in the literature. Structural studies have indicated possible binding sites in the SARS-CoV-2 RdRp for potential polymerase inhibitors (Jácome et al 2020, Gao et al 2020, Yin et al 2020, Hillen et al 2020, Elfiky 2020a). Given the high homology of the SARS-CoV and SARS-CoV-2 RdRp active site domains, it is likely that they will bind nucleotide analogues such as Sofosbuvir in a similar way, as we showed (Chien et al 2020). The structures of the SARS-CoV-2 RdRp nsp12 and its complex with nsp7 and nsp8 have been determined by cryo-EM (Gao et al 2020, Yin et al 2020), and these structures were compared with those of other RdRps including the SARS-CoV RdRp and HCV NS5B. These investigators performed docking studies to reveal likely binding sites for potential inhibitors and natural nucleotides. For instance, based on a docking study, Elfiky predicted that Ribavirin, Remdesivir, Sofosbuvir, Galidesivir, and Tenofovir may have inhibitory activity against SARS-CoV-2 RdRp (Elfiky 2020b). Gao et al. modeled Remdesivir diphosphate binding to SARS-CoV-2 nsp12 based on superposition with Sofosbuvir diphosphate bound to HCV NS5B, and found that the nsp12 of SARS-CoV-2 has the highest similarity with the Apo state of NS5B (Gao et al 2020). Yin et al. indicated that the orientations of the template-primer RNA in the active site of SARS-CoV-2 and hepatitis C virus NS5B are similar, and the amino acid residues involved in RNA binding and those making up the active site are highly conserved (Yin et al 2020).

By comparing the positive strand RNA genomes of HCV and SARS-CoV-2, Buonaguro et al. postulated that Sofosbuvir might be an optimal nucleotide analogue to repurpose for COVID-19 treatment (Buonaguro et al 2020). A detailed kinetic study of Remdesivir, Sofosbuvir and other nucleotide analogues indicated that Sofosbuvir triphosphate has an lower incorporation efficiency than the natural nucleotide (Gordon et al 2020b). Sofosbuvir in combination with Daclatasvir was recently shown to inhibit SARS-CoV-2 replication in Type II pneumocyte-derived (Calu-3) cells with an EC50 value of 0.7 µM (Sacramento et al 2020). Sofosbuvir was also reported to protect human brain organoids from SARS-CoV-2 infection (Mesci et al 2020).

After considering the potential advantages of Sofosbuvir including its low toxicity, its ability to be rapidly activated to the triphosphate form by cellular enzymes, and the high intracellular stability of this active molecule, COVID-19 clinical trials with EPCLUSA (a combination of Sofosbuvir and Velpatasvir) (Sayad et al 2020) and with Sofosbuvir plus Daclatasvir (World Hepatitis Alliance press release 2020) have been initiated in several countries. Recently, Sadeghi et al. reported promising results in a clinical trial using the combination drug Sofosbuvir (SOF or SFV) and Daclatasvir (DCV) to treat moderate or severe COVID-19 patients (Sadeghi et al 2020). These investigators showed that SOF/DCV treatment increased 14-day clinical recovery rates and reduced hospital stays. Two similar SOF/DCV clinical trials were also performed and provided preliminary evidence that this drug combination may have some benefit (Eslami et al 2020, Kasgari et al 2020).

Sofosbuvir and Velpatasvir together form the combination drug EPCLUSA, which is widely used for the treatment of HCV. Velpatasvir inhibits the viral replication protein NS5A in HCV (Gitto et al 2017, Quezada et al 2009); Daclatasvir also inhibits this protein (Smith et al 2016). Sacramento et al. reported that Daclatasvir was able to reduce SARS-CoV-2-induced enhancement of TNF-α and IL-6, which are key contributors to the cytokine storm (Sacramento et al 2020) . Because Velpatasvir and Daclatasvir have strong structural similarity and target the same NS5A protein in HCV, and Daclatasvir has also been shown to inhibit SARS-CoV-2 replication (Sacramento et al 2020) and is currently in COVID-19 clinical trial (World Hepatitis Alliance press release 2020); it is plausible that Velpatasvir will display similar inhibitory activity for SARS-CoV-2. Finally, Remdesivir has FDA approval (Eastman et al 2020), and is currently being tested for its safety and effectiveness in various COVID-19 clinical trials; in contrast, Sofosbuvir is an FDA-approved hepatitis C drug with wide availability and a well characterized safety and clinical profile.

### Repurposing of Drugs and Combination Drug Treatments

Our studies incorporated herein, coupled with further virological evaluation (Sacramento et al 2020), has led the clinical community to advance two groups of drugs, Sofosbuvir and the HCV NS5A inhibitors Velpatasvir/Daclatasvir, into COVID-19 clinical trials. The results of three initial studies suggest that the addition of Sofosbuvir and Daclatasvir to standard care may reduce the duration of hospital stays for COVID-19 patients compared to standard care alone (https://www.eurekalert.org/pub_releases/2020-08/oupu-sdm082220.php, Chan et al 2020, Eslami et al 2020). Combining the polymerase and exonuclease assays we have established, along with a reported helicase assay (Yuan et al 2020), the molecular mechanisms of several antivirals outlined below for inhibiting SARS-CoV-2 can be delineated. This will help to optimize the dosage for COVID-19 treatment.

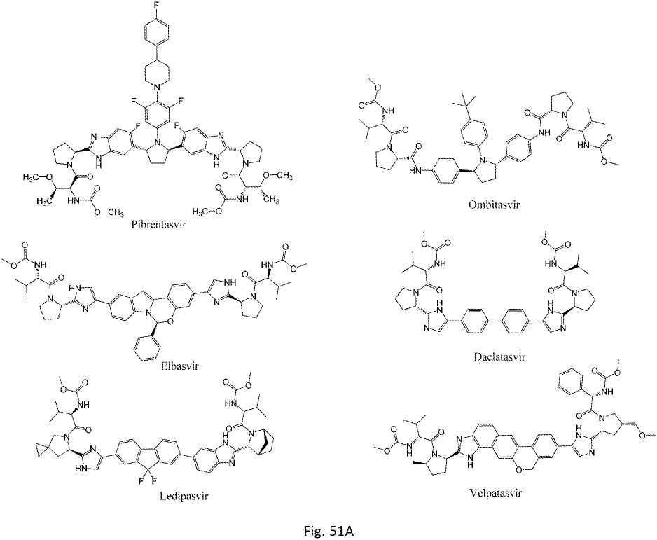

In HCV, the NS5A inhibitors prevent binding of RNA (Ascher et al 2014). While the target of Sofosbuvir is the SARS-CoV-2 polymerase, the target of the NS5A inhibitors for SARS-CoV-2 was unknown. We demonstrate herein that Daclatasvir and Velpatasvir both inhibit RdRp activity. Besides Velpatasvir and Daclatasvir, there are four additional FDA-approved oral HCV NS5A inhibitors with similar core structures (Ledipasvir, Ombitasvir, Elbasvir and Pibrentasvir) (FIG. 51A). We have evaluated members of this group of NS5A inhibitors in our polymerase and exonuclease assays, and inhibitory activity was found for both of these enzyme activities (results shown below). Recently it has been shown that HCV NS3/4a inhibitors also play a role in inhibiting SARS-CoV-2 in lung cells (Nguyenla et al 2020); NS3 is a serine protease and NS4A is an NS3 cofactor with additional described functions. These investigators also indicated that the combination of EPCLUSA (Sofosbuvir/Velpatasvir) with Remdesivir increased the potency of

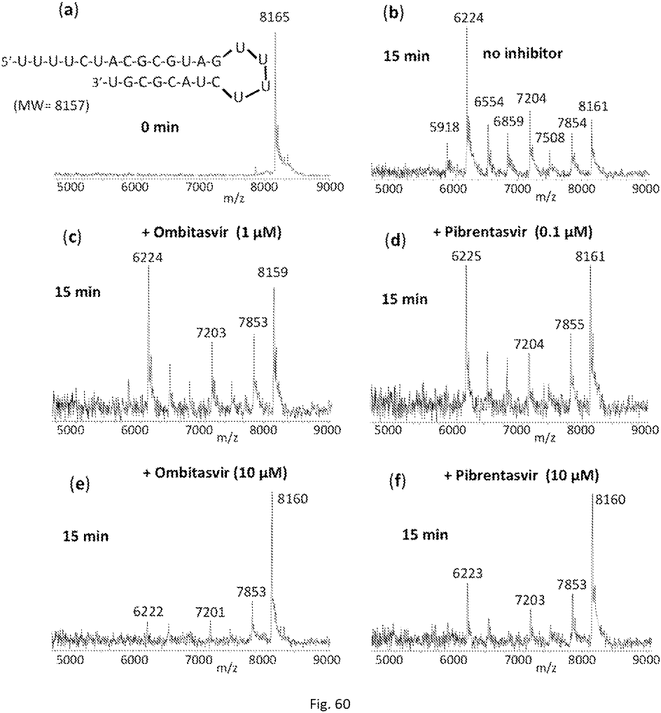

Remdesivir 25-fold. There are six FDA-approved oral HCV NS3/4a inhibitors with similar core structures (Paritaprevir, Glecaprevir, Voxilaprevir, Grazoprevir, Danoprevir and Telaprevir) (FIG. 61). They can be combined with drugs already known to target polymerase, exonuclease, helicase and protease activities of SARS-CoV-2.

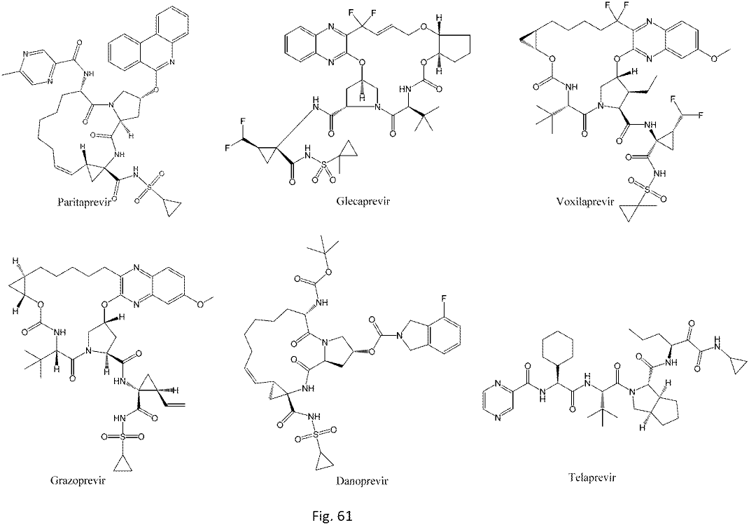

Recently, the FDA-approved HIV integrase inhibitor Raltegravir (FIG. 62) has been shown to inhibit the SARS-CoV-2 exonuclease (Baddock et al 2020). There are three additional FDA-approved oral integrase inhibitors with similar core structures (Bictegravir, Dolutegravir and Elvitegravir) (FIG. 62). We show below that Elvitegravir displays SARS-CoV-2 exonuclease inhibition, and thus this class of inhibitors may be used in combination with other drugs that inhibit the SARS-CoV-2 RdRp, helicase, proteases, and other viral proteins.

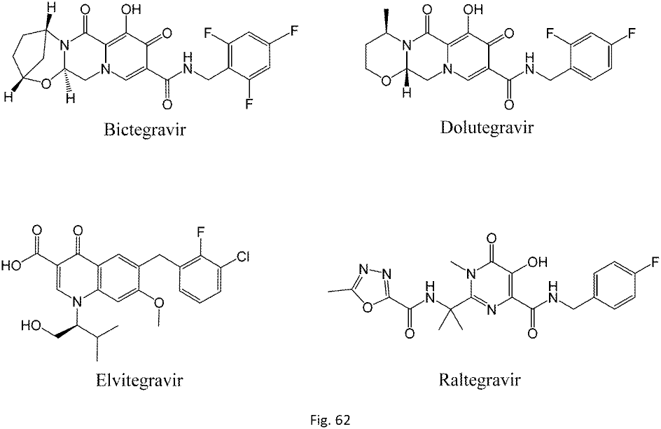

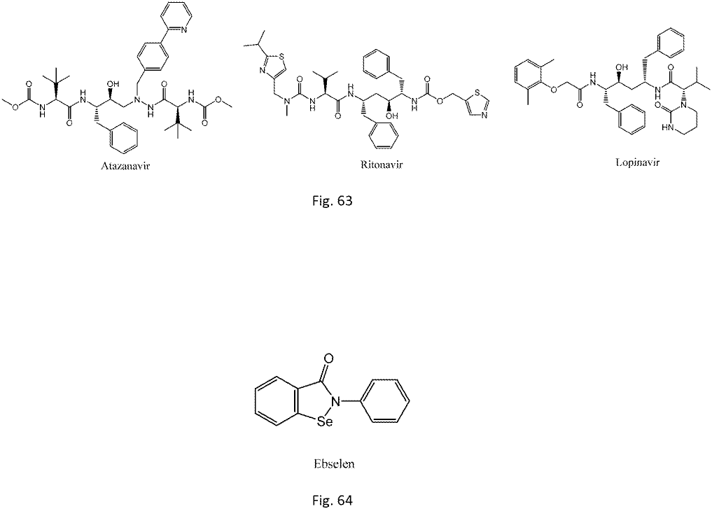

The drug Ritonavir, a known protease inhibitor approved by the FDA for the treatment of HIV/AIDS (FIG. 63), has recently been suggested by docking studies to inhibit SARS-CoV-2 exonuclease activity by preventing binding of RNA (Narayanan & Nair 2021), and the authors suggest that it can be possibly used in combination with drugs such as Remdesivir, Favipiravir and Ribavirin for COVID-19. However, the computational approach has high uncertainty, and requires biological experiments to confirm the activity. Using AI technology and experimental verification, an examination of 12 drugs in 53,000 combinations led to the recommendation of an optimized combination therapy consisting of Remdesivir, Ritonavir and Lopinavir (a viral protease inhibitor that works in combination with Ritonavir, FIG. 63) (Blasiak et al 2020). However, the mechanism of Ritonavir and Lopinavir for targeting SARS-CoV-2 was unknown. Herein, we demonstrate that Ritonavir and Lopinavir inhibit the SARS-CoV-2 exonuclease. A clinical trial has been carried out involving treatment of patients early in the course of mild or moderate infections with interferon beta-1b, Lopinavir-Ritonavir and Ribavirin (Hung et al 2020). Compared to a control group receiving Lopinavir-Ritonavir, these patients had a shorter time to negative viral loads and virus shedding, reduced cytokine response, and earlier discharge from the hospital. Another study, after screening for synergism of 1200 drugs with Remdesivir in inhibiting viral production in Calu-3 lung cells, found the strongest effects with the NS5A inhibitors Velpatasvir or Elbasvir, with even more pronounced effects when combined with their pharmaceutical partners, Sofosbuvir in EPCLUSA and Grazoprevir in Zepatier, respectively (Nguyenla et al 2020). A CryoEM study indicated that Suramin inhibited the SARS-CoV-2 RdRp by blocking binding of RNA and the incoming NTP to the active site, and the authors then demonstrated it was 20× more potent than Remdesivir in biochemical assays, and that it inhibited replication in cell culture (Yin et al 2020). Ranitidine bismuth citrate has been shown to inhibit both the ATPase and unwinding activities of the SARS-CoV-2 helicase via displacement of Zn ions by Bi ions, to protect virus-infected cells and decrease viral loads in the respiratory tract of hamsters (Yuan et al 2020). A Nigerian trial combining the anti-parasitic Nitazoxanide and the HIV protease inhibitors Atazanavir and Ritonavir has been proposed (Olagunju et al 2021) and a docking study has shown that atazanavir binds to the active site of the SARS-CoV-2 major protease M^{pro}, inhibits viral production in Vero and pulmonary epithelial cell culture in the presence or absence of Ritonavir, and that Atazanavir/Ritonavir blocks production of IL-6 and TNF-alpha (Fintelman-Rodrigues et al 2020). Baddock et al (2020) recently demonstrated that the protease inhibitor Ebselen also inhibits the SARS-CoV-2 exonuclease.

Thus, combinations of a variety of the polymerase, exonuclease, helicase and protease inhibitors described herein are candidates for repurposing for prevention and/or treatment of COVID-19, as well as other coronavirus infections such as SARS and MERS, and indeed other viruses including but not limited to Zika, Ebola and Marburg virus.

### EXAMPLES

Various features and embodiments of the disclosure are illustrated in the following representative examples, which are intended to be illustrative, and not limiting. Every embodiment and feature described in the application should be understood to be interchangeable and combinable with every embodiment contained within.

In the following examples, we describe several types of inhibitors that have the ability to block coronavirus replication. The first group of compounds, described in Examples 1-5, are nucleoside triphosphates that can be incorporated into RNA where they serve as terminators of the polymerase reaction. We provide examples for which we have demonstrated inhibition of the RdRp from SARS-CoV (Examples 3 and 5) and SARS-CoV-2 (Examples 4 and 5) using polymerase catalyzed extension reactions with MALDI-TOF MS-based detection of the extension products. In some cases, these terminators, once incorporated into RNA, show resistance to excision by the SARS-CoV-2 exonuclease (Example 6), as determined by exonuclease assays with MS detection. These include Sofosbuvir, which is significantly more resistant to removal by the exonuclease than Remdesivir (Example 7). The next group of compounds are non-nucleoside, non-nucleotide inhibitors of the SARS-CoV-2 RdRp (Example 8). These are not incorporated into the RNA but still inhibit the polymerase reaction, again demonstrated using polymerase catalyzed extension assays. And finally, the last group of compounds indirectly or directly inhibit the SARS-CoV-2 exonuclease (Example 9), as demonstrated using the above exonuclease assay.

The results also are included in the following publications which are herein incorporated by reference (Ju et al 2020a,b, Chien et al 2020 a,b, Jockusch et al 2020a-d).

### Example 1: 2′-F,Me-UTP, the Active Triphosphate of Sofosbuvir, is Incorporated into DNA by Low Fidelity Polymerases and Terminates the Polymerase Reaction

The active triphosphate form of Sofosbuvir, 2′-F,Me-UTP, was shown to be incorporated by HCV RdRp and prevent any further incorporation by this polymerase (Fung et al 2014, Deval et al 2014). Other viral polymerases have also been shown to incorporate active forms of various anti-viral prodrugs to cause termination of further replication (Fearns & Deval 2016). We selected two groups of polymerases to test the termination effectiveness of the active form of Sofosbuvir, one group with high fidelity, mimicking host cell polymerases, and one group with low fidelity, to mimic viral polymerases. Our rationale is that the low fidelity viral-like enzymes would incorporate 2′-F,Me-UTP and stop further polymerase reaction, while the high fidelity polymerases, mimicking host cell polymerases, will not incorporate this activated nucleotide analogue.

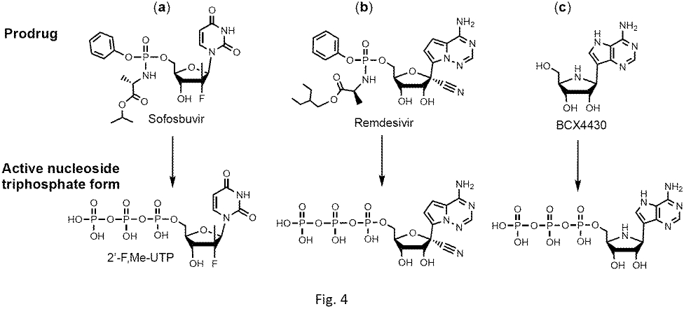

Based on this rationale, we carried out DNA polymerase extension reactions with 2′-F,Me-UTP using Thermo Sequenase as an example of high fidelity, host-like polymerases, and two mutated DNA polymerases which are known to be more promiscuous in their ability to incorporate modified nucleotides, Therminator II and Therminator IX, as examples of viral-like low fidelity enzymes. A DNA template-primer complex, in which the next two available bases were A, was incubated with either 2′-F,Me-UTP (structure shown in FIG. 4a), or dTTP as a positive control, in the appropriate polymerase buffer. The 2′-F,Me-UTP, if incorporated, should result in primer extension by a single base, since the incorporated nucleotide analogue should inhibit further incorporation. In contrast, dTTP incorporation will result in primer extension by 2 bases. After performing the reactions, we determined the molecular weight of the extension products using MALDI-TOF-mass spectrometry.

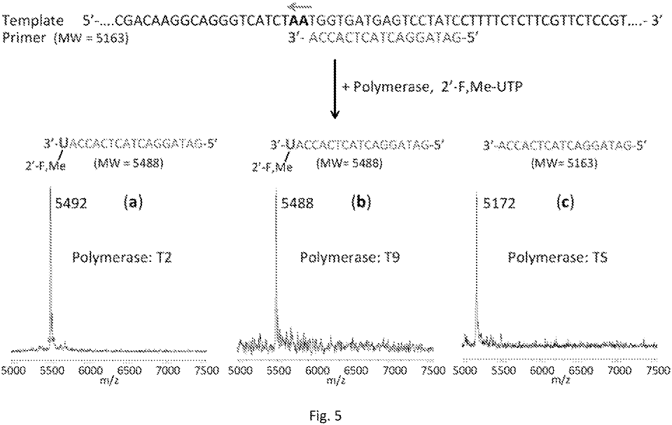

The detailed method is as follows: Oligonucleotides were purchased from Integrated DNA Technologies. The 20 µl extension reactions consisted of 3 µM DNA template and 5 µM DNA primer (sequences shown in FIG. 5), 10 µM 2′-F,Me-UTP (Sierra Bioresearch) or 10 µM dTTP, 1× Thermo Sequenase buffer or 1× ThermoPol buffer (for Therminator enzymes), and either 10 U Thermo Sequenase (GE Healthcare), 4 U Therminator II (New England Biolabs) or 10 U Therminator IX (New England Biolabs). (The 1× Thermo Sequenase buffer consists of 26 mM Tris-HCl, pH 9.5 and 6.5 mM MgCl₂. The 1× ThermoPol buffer contains 20 mM Tris-HCl, pH 8.8, 10 mM (NH₄)₂SO₄, 10 mM KCl, 2 mM MgSO₄, and 0.1% Triton X-100.) Incubations were performed in a thermal cycler using 15 cycles of 30” at 65° C., 30” at 45° C. and 30” at 65° C. Following desalting using an Oligo Clean & Concentrator (Zymo Research), the samples were subjected to MALDI-TOF-MS (Bruker ultrafleXtreme).

As seen in FIGS. 5a and b, when the primer-template complex (sequences shown at top of FIG. 5) and the 2′-F,Me-UTP, were incubated with the promiscuous (low fidelity) 9°N polymerase mutants, Therminator II (T2) and Therminator IX (T9), we observed single product peaks with MW’s of 5492 Da and 5488 Da (5488 Da expected), indicating single base extension in the polymerase reaction. Thus the 2′-F,Me-UTP was able to be incorporated and block further nucleotide incorporation. In contrast, when the extension reactions were carried out with Thermo Sequenase DNA polymerase (TS), there was no incorporation, as evidenced by a single primer peak at 5172 Da (expected 5163 Da, within instrument error of ± 10 Da) (FIG. 5c). This was expected given the fact that Thermo Sequenase is a high fidelity enzyme originally designed for accurate Sanger sequencing (Tabor & Richardson 1995). When dTTP was used as a positive control with these three enzymes, incorporation continued past the first A in the template, resulting in a higher molecular weight peak.

These results demonstrate that lower fidelity polymerases, of which the viral RdRp is an example, will incorporate 2′-F,Me-UTP and inhibit viral RNA replication, whereas high fidelity enzymes, more typical of the host DNA and RNA polymerases, will have a low likelihood of being inhibited by 2′-F,Me-UTP.

### Example 2: Design of Viral Polymerase Inhibitors With 3′ Blocking Groups

Based on the above analysis and results, we describe here a novel strategy to design and synthesize viral polymerase inhibitors, by combining the ProTide Prodrug approach used in the development of Sofosbuvir with the use of 3′-blocking groups that we have built into nucleotide analogues that function as reversible terminators for DNA sequencing (Ju et al 2003, Ju et al 2006, Guo et al 2008). We reasoned that (i) the phosphate masking groups will allow entry of the compounds into infected cells, (ii) the 3′-blocking group on the 3′-OH with either free 2′-OH or modifications at the 2′ position will encourage incorporation of the activated triphosphate analogue by viral polymerases but not host cell polymerases, thus reducing any side effects, and (iii) once incorporated, further extension will be prevented by virtue of the 3′-blocking group, thereby completely inhibiting viral replication. The latter point has important consequences with regard to mutability of the polymerase, since even Sofosbuvir can select for mutations in the RdRp that reduce its effectiveness during infections (Xu et al 2017). In addition to phosphate masking groups, the presence of hydrophobic blocking groups at the 3′ position will further enhance the ability of these drugs to enter the virus-infected cells. These modified nucleotide analogues should be potent polymerase inhibitors and thus active against various viral diseases, including but not limited to the coronaviruses such as SARS-CoV-2, and the strains causing SARS and MERS. Once incorporated, our newly designed nucleotide analogues containing 3′ blocking groups will permanently block further viral genome replication. This is in contrast to other nucleotide analogue-based viral inhibitors that have a free 3′ OH group, which have the possibility of allowing further polymerase extension, enabled by viral mutations.

The rational selection of a 3′-blocking group should also aim to increase the overall selectivity of these nucleotides for the viral RdRp versus the host DNA and RNA polymerases. Other FDA-approved nucleoside analogues that target viral polymerases have very small substituents at the 3′ position (e.g., 3′-azido dTTP: AZT), which can also allow them to be incorporated by both viral and host polymerases including mitochondrial polymerases, causing polymerase reaction termination and resulting toxicity (Margolis et al 2014); thus, they are replication terminators for both the viral and host polymerases. All RNA viruses are known to mutate at a high frequency, due to the low fidelity of the viral polymerase, resulting in the development of resistance to treatment (Dutartre et al 2006). We reasoned that the promiscuous nature of the viral polymerase will allow incorporation of our newly designed nucleotide analogues, while the host polymerase will not incorporate these molecules even at high concentration. This approach has the potential to lead to the development of a new class of anti-viral agents with fewer side effects. Modifications on the base or phosphate moiety of nucleotide analogues are generally tolerated better by polymerases than those on the sugar (Kumar et al 2005, Sood et al 2005). Our design criterion is to identify groups for attachment to the 3′-OH with appropriate structural and chemical properties (e.g., size, shape, rigidity, flexibility, polarity, reactivity (Ju et al 2003, Canard & Sarfati 1994)), along with appropriate 2′-substitutions, so that they will be incorporated by the viral RdRp but not the host polymerases. In addition, unlike the masking group on the phosphate moiety, they should not be cleaved by viral or host esterases (Ju et al 2006).

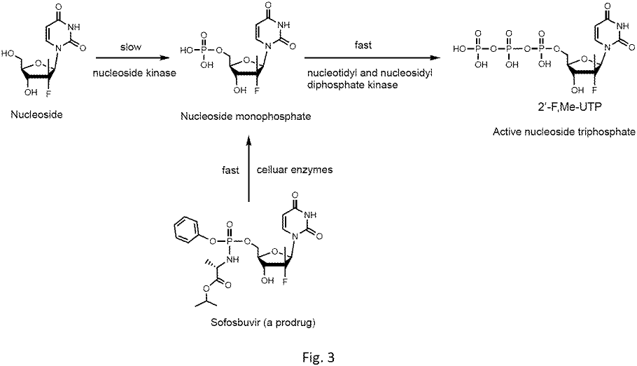

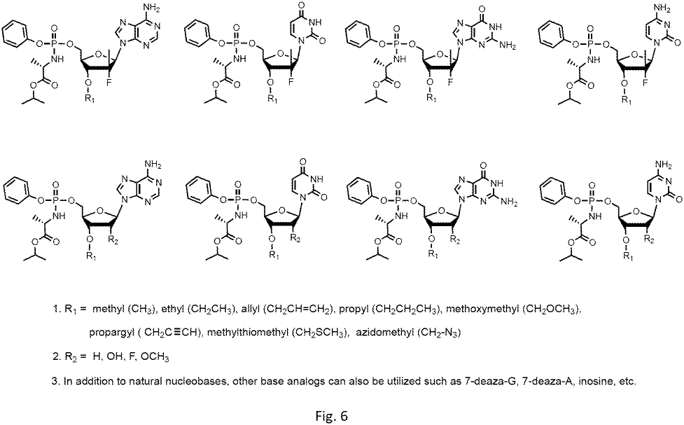

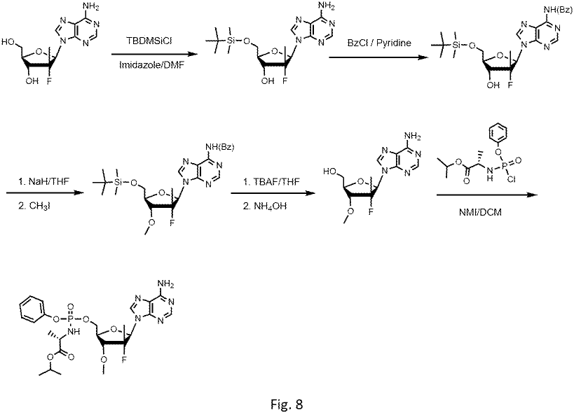

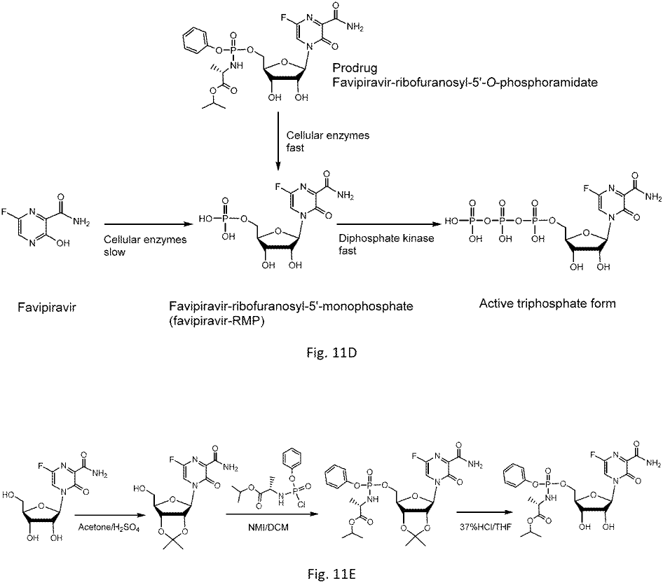

Examples of nucleotide analogues we designed to satisfy these criteria are provided in FIGS. 6 and 7, and strategies for their synthesis in FIGS. 8-10; FIG. 11A shows the activation of these prodrugs to form triphosphate analogues (in the same way as Sofosbuvir in FIG. 3), which should be incorporated into the RNA and inhibit the coronavirus and other RNA virus polymerases.

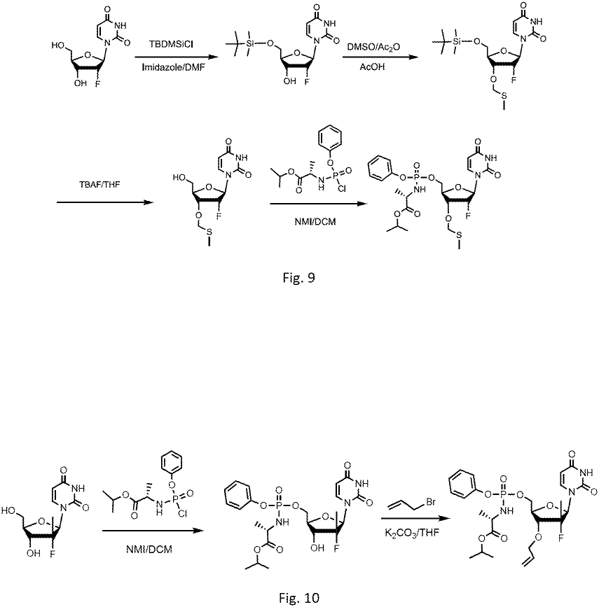

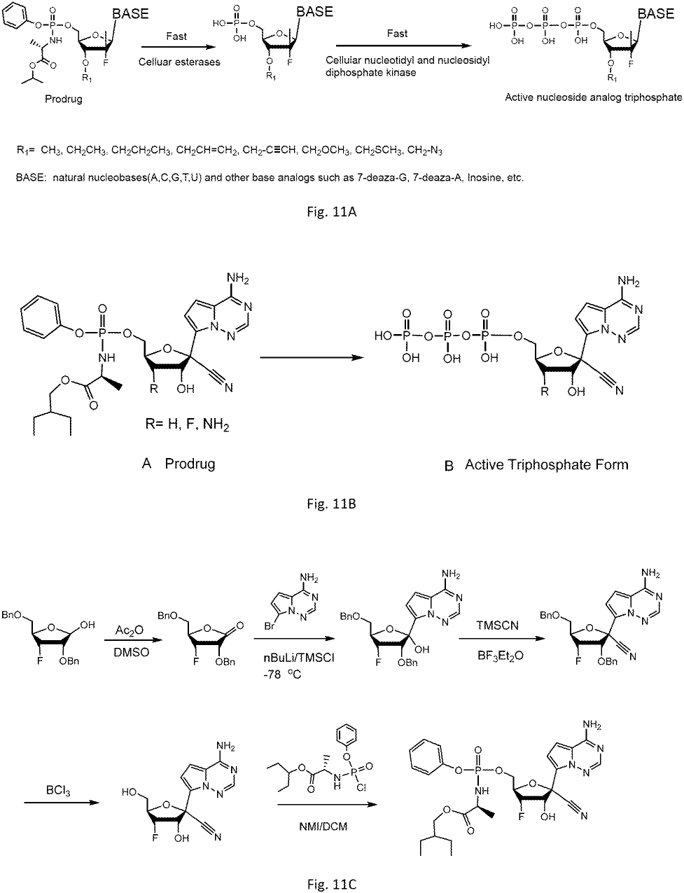

Synthesis of 3′-O-blocked nucleoside phosphoramidate prodrugs can be carried out starting from 2′-modified nucleosides (Ross et al 2011). In a typical approach, first, both the 5′-OH and the exocyclic amino group of the base will be protected. Then the 3′-OH will be derivatized with a variety of blocking groups, including methyl, ethyl, propyl, allyl, propargyl, methoxymethyl, methylthiomethyl, azidomethyl, etc., such as those listed in FIG. 6, following established methods (Ju et al 2006, Guo et al 2008). After deprotection, the free 5′-OH is derivatized to afford the corresponding phosphoramidates by treatment with freshly prepared chlorophosphoramidate reagent in the presence of N-methyl imidazole (Ju et al 2003). FIG. 8 and FIG. 9 show example synthetic routes for the 3′-methoxy and 3′-O-methylthiomethyl nucleoside phosphoramidate analogs, respectively. Alternatively, starting from a 2′-modified nucleoside, the 5′-OH can be derivatized first to give 5′-phosphoramidate nucleotides, followed by 3′-OH derivatization to afford 3′-O blocked nucleoside phosphoramidate analogues. FIG. 10 shows an example synthetic route for 3′-allyl nucleoside phosphoramidate analogues.

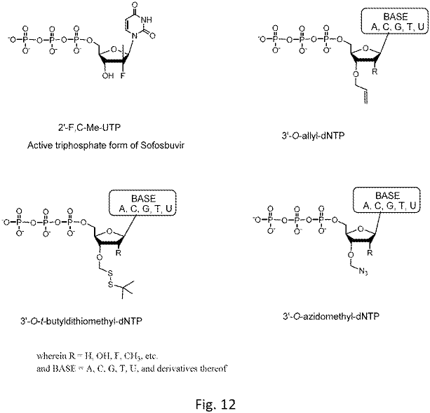

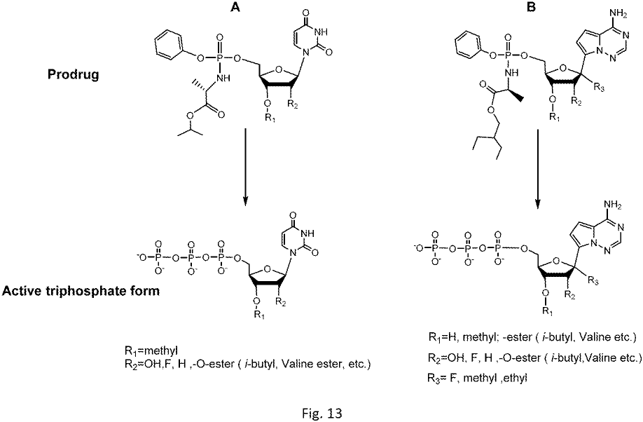

Other nucleotide analogues that can be potent inhibitors of coronavirus and other RNA virus RdRp’s comprise the compounds illustrated in FIGS. 12-13. In the case of Remdesivir analogues, the replacement of the cyano group in Remdesivir at the 1′ position with smaller moieties including those shown in FIG. 13B will increase the efficiency of incorporation and then termination of the polymerase reaction, thereby completely inhibiting viral replication.

Remdesivir is not an immediate terminator; rather it shows delayed termination. The structure of Remdesivir can be modified by placing small moieties at the 3′ position (e.g., fluoro or amino) which will still allow efficient incorporation, but will stop further RdRp-catalyzed RNA synthesis. These novel compounds will still undergo in vivo conversion to the active triphosphate forms (FIG. 11B). An example chemical synthetic scheme for 1′-cyano-3′-F-s-4-aza-7,9-dideaza-adenosine C-nucleotide phosphoramidate is presented in FIG. 11C. Benzyl protected 3-deoxy-3-fluoro-β-D-ribofuranose can be readily synthesized or obtained commercially. After oxidation to its ketone form, 9-bromo-4-aza-7,9-dideazaadenosine is used to form C-nucleoside, then the resulting 1′-OH is substituted with a cyano group affording 1′-cyano-3′-F-4-aza-7,9-dideazaadenosine C-nucleosides. The following de-protection will generate free 2′ and 3′ hydroxyl groups, and further treatment with freshly prepared chlorophosphoramidate reagent in the presence of N-methyl imidazole (Ross et al 2011, Sofia et al 2010) will yield the 3′-F-nucleotide phosphoramidate prodrug.

Favipiravir is another drug that is that can inhibit viral RdRps and has been used to treat novel influenza strains. It is converted by cellular enzymes to Favipravir-ribofuranosyl-5′-monophosphate (Favipiravir-RMP) and then the active triphosphate form (Favipiravir-RTP), which can be incorporated into RNA by the viral RdRp (FIG. 11D). A ProTide type prodrug (top of figure) which would have better stability extracellularly and still be metabolized to the active triphosphate form upon entry into cells can be synthesized using the pathway shown in FIG. 11E.

### Example 3: Incorporation of Nucleotide Analogues Into RNA by SARS-CoV RdRp and Their Evaluation as Terminators of the Polymerase Reaction

We tested the ability of the activated (triphosphate) form of Sofosbuvir, 2′-F,Me-UTP, and a different nucleotide analogue, 3′-fluoro-3′-deoxythymidine triphosphate (3′-F-dTTP), to be incorporated by an RNA-dependent RNA polymerase (RdRp). We used the RdRp of SARS-CoV (responsible for the 2003 SARS outbreak), referred to as nsp12, and its two viral cofactors, nsp7 and nsp8, shown to be required for the processive polymerase activity of nsp12 (Subissi et al 2014, Kirchdoerfer & Ward 2019). These three viral gene products have high homology at the amino acid level (e.g., 96% identity and 98% similarity for nsp12, with similar homology levels for nsp7 and nsp8) to the equivalent gene products from SARS-CoV-2 (the causative agent of the recent COVID-19 outbreak).

Like Sofosbuvir, the prodrug form of 2′-F,Me-UTP (FIG. 14A), 3′-F-dTTP can also be synthesized in prodrug form (FIG. 14B). Synthesis of 5′-0-phosphoramidate nucleoside prodrugs can be carried out starting from 2′ or 3′-modified nucleosides, respectively. In a typical approach, the 5′-OH is derivatized to afford the corresponding phosphoramidates by treatment with freshly prepared chlorophosphoramidate reagent in the presence of N-methyl imidazole (Ross et al 2011, Sofia et al 2010).

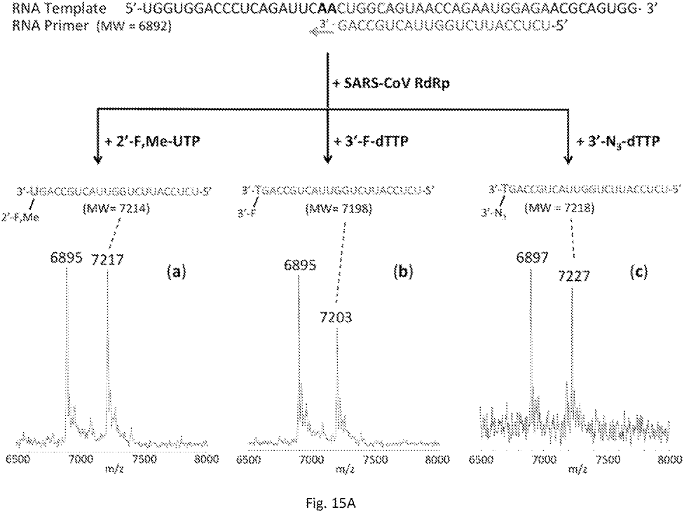

We performed polymerase extension assays with 2′-F,Me-UTP, 3′-F-dTTP, 3′-N₃-dTTP or UTP following the addition of a pre-annealed RNA template and primer to a pre-assembled mixture of the RdRp (nsp12) and two cofactor proteins (nsp7 and nsp8). The extended primer products from the reaction were subjected to MALDI-TOF-MS analysis. The RNA template and primer, corresponding to the N1 epitope region of the N protein of the SARS-CoV-2 virus, were used for the polymerase assay, and their sequences are indicated at the top of FIGS. 15A and 15B.

The detailed method is as follows: Oligonucleotides were purchased from IDT, Inc. Following a published strategy (Subissi et al 2014, Kirchdoerfer & Ward 2019), the primer and template (sequences shown in FIGS. 15A and 15B) were annealed by heating to 70° C. for 10 min and cooling to room temperature in 1× reaction buffer. The RNA polymerase mixture consisting of 2 µM nsp12 and 6 µM each of cofactors nsp7 and nsp8 was incubated for 15 min at room temperature in a 1:3:3 ratio in 1× reaction buffer. Then 5 µl of the annealed template primer solution containing 2 µM template and 1.7 µM primer in 1× reaction buffer was added to 10 µl of the RNA polymerase mixture and incubated for an additional 10 min at room temperature. Finally, 5 µl of a solution containing either 2 mM 2′-F,Me-UTP, 2 mM 3′-F-dTTP, 2 mM 3′-N₃-dTTP or 2 mM UTP in 1× reaction buffer was added, and incubation was carried out for 2 hr at 30° C. The final concentrations of reagents in the 20 µl extension reactions were 1 µM nsp12, 3 µM nsp7, 3 µM nsp8, 425 nM RNA primer, 500 nM RNA template, either 500 µM 2′-F,Me-UTP (Sierra Bioresearch), 500 µM 3′-F-dTTP (Amersham Life Sciences), or 500 µM 3′-N₃-dTTP (Amersham Life Sciences), and 1× reaction buffer (10 mM Tris-HCl pH 8, 10 mM KCl, 2 mM MgCl₂ and 1 mM β-mercaptoethanol). In the experiment with UTP shown in Supplementary FIG. 15B, the final concentrations were 500 nM nsp12, 1.5 µM nsp7, 1.5 µM nsp8, 425 nM RNA primer, 250 nM RNA template and 500 µM UTP (Fisher) and the reaction time was 1 h at 30° C. Following desalting using an Oligo Clean & Concentrator (Zymo Research), the samples were subjected to MALDI-TOF-MS (Bruker ultrafleXtreme) analysis.

Because there are two As in a row in the next available positions of the template for RNA polymerase extension downstream of the priming site, if 2′-F,Me-UTP, 3′-F-dTTP or 3′-N₃-dTTP are incorporated by the viral RdRp, the nucleotide analogue will be added to the 3′-end of the primer strand. If they are indeed inhibitors of the polymerase, the extension should stop after this incorporation; further 3′-extension should be prevented. In the case of the UTP control reaction, two UTPs should be incorporated. As shown in FIGS. 15A and 15B, this is exactly what we observed. In the MALDI-TOF MS trace in FIG. 15A(a), a peak indicative of the molecular weight of a primer extension product terminated with one 2′-F,Me-UTP was obtained (7217 Da observed, 7214 Da expected). Similarly, in the trace in FIG. 15A(b), a single extension peak indicative of a single-base extension product terminated by 3′-F-dTTP is revealed (7203 Da observed, 7198 Da expected), with no further incorporation. And in the trace in FIG. 15A(c), a single extension peak indicative of a single-base extension by 3′-N₃-dTTP is seen (7227 Da observed, 7218 Da expected), with no evidence of further incorporation. As a positive control, primer extension by 2 UTPs occurred (7506 Da observed, 7504 Da expected) as shown in the MALDI-TOF MS trace in FIG. 15B.

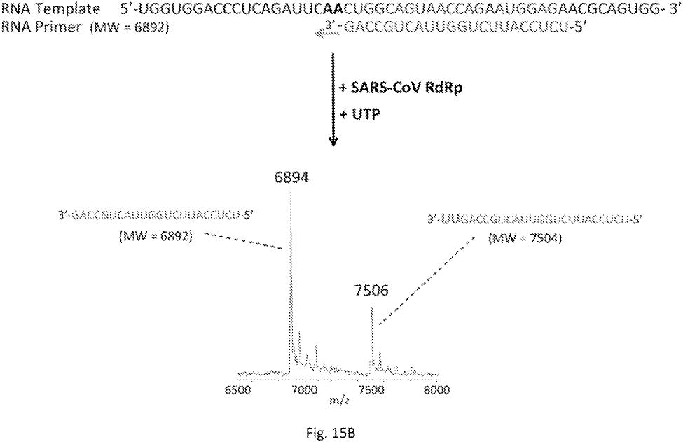

In summary, these results demonstrate that the nucleotide analogues 2′-F,Me-UTP, 3′-F-dTTP and 3′-N₃-dTTP, are permanent terminators for the SARS-CoV RdRp. Their prodrug versions (Sofosbuvir, 3′-F-5′-O-phosphoramidate dT nucleoside and 3′-N₃-5′-O-phosphoramidate dT nucleoside, shown in FIGS. 7a, c, d and FIG. 16) can be readily synthesized using the ProTide prodrug approach, and can be evaluated as potential therapeutics for both SARS and COVID-19.

A prodrug form of 3′-N₃-dTTP can be synthesized as follows. Synthesis of a 5′-O-phosphoramidate nucleoside prodrug (3′-azido-5′-O-phosphoramidate-dT) can be carried out directly from AZT. In a typical approach, the 5′-OH of AZT is derivatized to afford the corresponding phosphoramidate by treatment with freshly prepared chlorophosphoramidate reagent in the presence of N-methyl imidazole (Ross et al 2011, Sofia et al 2010).

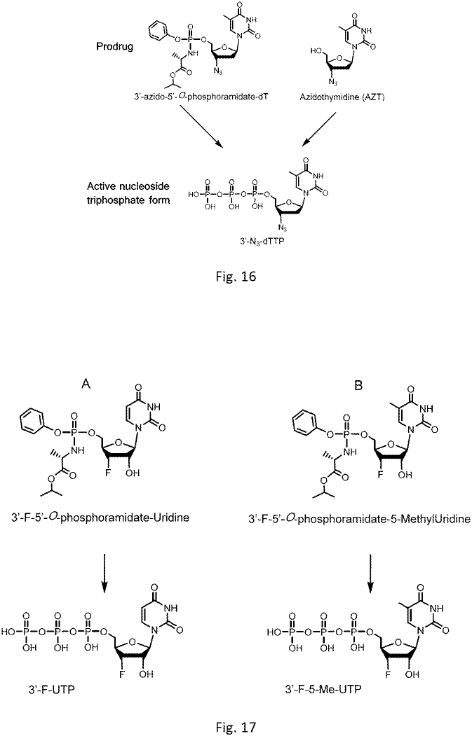

Two other designed uridine-based analogues that can be synthesized and evaluated as polymerase terminators and their precursors are shown in FIG. 17.

### Example 4: Evaluation of the Active Triphosphate Forms of Sofosbuvir, Alovudine, AZT, Tenofovir Alafenamide, Emtricitabine, Lamivudine and Carbovir as Terminators of the SARS-CoV-2 RdRp Reaction

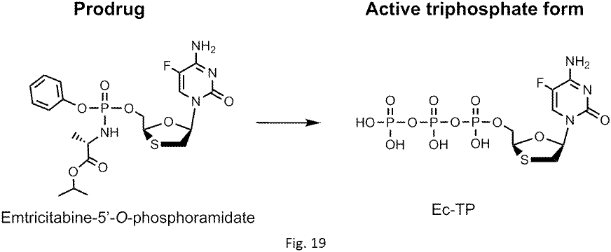

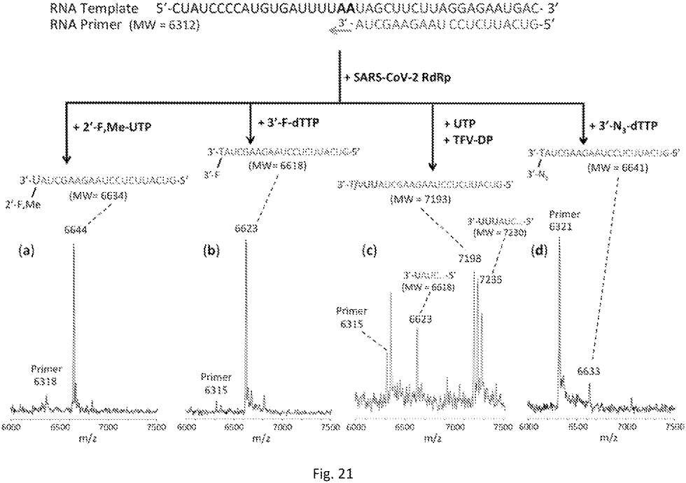

Structures of the compounds to be tested are shown in FIG. 7 and FIGS. 18-20. For the polymerase extension assay we used the pre-assembled RNA template and primer, corresponding to the 3′ end of the SARS-CoV-2 genome; their sequences are indicated at the top of FIGS. 21-23. The detailed protocols for the extension assays are as follows:

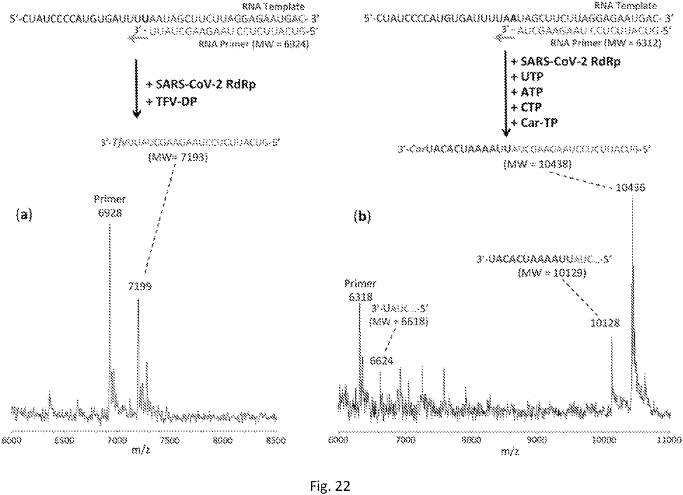

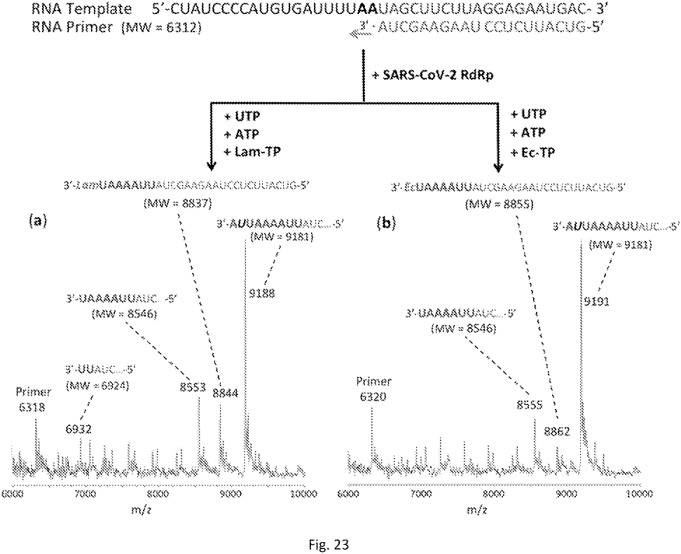

Extension reactions with SARS-CoV-2 RNA-dependent RNA polymerase. The SARS-CoV-2 polymerase nsp12 and its cofactors nsp7 and nsp8 were cloned and expressed as described in Chien et al (2020a, b). The RNA primers and template (sequences shown in FIGS. 21-23) were annealed by heating to 70° C. for 10 min and cooling to room temperature in 1× reaction buffer. For reactions in FIG. 21, the RNA polymerase mixtures consisting of 6 µM nsp12 and 18 µM each of cofactors nsp7 and nsp8 were incubated for 15 min at room temperature in a 1:3:3 ratio in 1× reaction buffer. For reactions in FIGS. 22-23, higher concentrations of nsp 12, nsp7 and nsp8 were used (10 µM, 30 µM and 60 µM, respectively). Then 5 µl of the annealed template primer solution containing 2 µM template and 1.7 µM primer in 1× reaction buffer was added to 10 µl of the RNA polymerase mixture and incubated for an additional 10 min at room temperature. Finally, 5 µl of a solution containing either 2 mM 2′-F,Me-UTP (FIG. 21a), 2 mM 3′-F-dTTP (FIG. 21b), 2 mM TFV-DP + 200 µM UTP (FIG. 21c), 2 mM 3′-N3-dTTP (FIG. 21d), 2 mM TFV-DP (FIG. 22a), 400 µM UTP + 400 µM ATP + 400 µM CTP + 1 mM Car-TP (FIG. 22b), 400 µM UTP + 400 µM ATP + 2 mM Lam-TP (FIG. 23a) or 400 µM UTP + 400 µM ATP + 2 mM Ec-TP (FIG. 23b) in 1× reaction buffer was added, and incubation was carried out for 2 hrs at 30° C. The final concentrations of reagents in the 20 µl extension reactions were 3 µM nsp12, 9 µM nsp7, 9 µM nsp8 (FIG. 21) or 5 µM nsp12, 15 µM nsp7, 30 µM nsp8 (FIGS. 22 and 23), 425 nM RNA primer, 500 nM RNA template, and either 500 µM 2′-F,Me-UTP (FIG. 21a), 500 µM 3′-F-dTTP (FIG. 21b), 500 µM TFV-DP + 50 µM UTP (FIG. 21c), 500 µM 3′-N3-dTTP (FIG. 21d), 500 µM TFV-DP (FIG. 22a), 100 µM UTP + 100 µM ATP + 100 µM CTP + 250 µM Car-TP (FIG. 22b), 100 µM UTP + 100 µM ATP + 500 µM Lam-TP (FIG. 23a) or 100 µM UTP + 100 µM ATP + 500 µM Ec-TP (FIG. 23b). The 1× reaction buffer contains the following reagents: 10 mM Tris-HCl pH 8, 10 mM KCl, 2 mM MgCl₂ and 1 mM β-mercaptoethanol. Following desalting using an Oligo Clean & Concentrator (Zymo Research), the samples were subjected to MALDI-TOF-MS (Bruker ultrafleXtreme) analysis.

Given the 98% amino acid similarity of the SARS-CoV and SARS-CoV-2 RdRps and our previous inhibition results on SARS-CoV and SARS-CoV-2 RdRps (Ju et al 2020b, Jockusch et al 2020a) we reasoned that the nucleotide analogues listed in FIGS. 7 and 18-20 should also inhibit the SARS-CoV-2 polymerase. We thus assessed the ability of 2′-F,Me-UTP, 3′-F-dTTP, TFV-DP, and 3′-N₃-dTTP (the active triphosphate forms of Sofosbuvir, Alovudine, TAF and AZT, respectively), along with Lam-TP, Ec-TP and Car-TP (the active triphosphate forms of Lamivudine, Emtricitabine and Carbovir/Abacavir), to be incorporated by SARS-CoV-2 RdRp into an RNA primer to terminate the polymerase reaction.

We performed polymerase extension assays with 2′-F,Me-UTP, 3′-F-dTTP, 3′-N₃-dTTP or TFV-DP + UTP, following the addition of a pre-annealed RNA template and primer to a pre-assembled mixture of the SARS-CoV-2 RdRp (nsp12) and two cofactor proteins (nsp7 and nsp8) . The primer extension products from the reaction were subjected to MALDI-TOF-MS analysis. The RNA template and primer, corresponding to the 3′ end of the SARS-CoV-2 genome, were used for the polymerase reaction assay; their sequences are indicated at the top of FIG. 21. 2′-F,Me-UTP has a 3′-OH group, but due to 2′ modification with a fluorine and methyl group, it acts as a non-obligate terminator for HCV RdRp (Eltahla et al 2015). 3′-F-dTTP and 3′-N₃-dTTP don’t have a 3′-OH, and we previously demonstrated that they are obligate terminators of the SARS-CoV RdRp (Ju et al 2020b).

For the data presented in FIG. 21, because there are two As in a row in the next available positions of the template for RNA polymerase extension downstream of the priming site, if 2′-F,Me-UTP, 3′-F-dTTP or 3′-N₃-dTTP are incorporated by the viral RdRp and terminate the polymerase reaction, a single nucleotide analogue will be added to the 3′-end of the primer strand. Because the two As in the template are followed by four Us, in the case of the TFV-DP/UTP mixture, two UTPs should be incorporated prior to the incorporation and termination by TFV-DP, which is an ATP analogue and an obligate terminator due to the absence of an OH group. As shown in FIG. 21, this is exactly what we observed. In the MALDI-TOF MS trace in FIG. 21a, a peak indicative of the molecular weight of a single nucleotide (2′-F,Me-UMP) primer extension product was obtained (6644 Da observed, 6634 Da expected). Similarly, in the trace in FIG. 21b, a single extension peak indicative of a single base extension by 3′-F-dTMP is revealed (6623 Da observed, 6618 Da expected), with no further incorporation. In both of the above cases, the primer was nearly completely depleted, indicating that 2′-F,Me-UTP and 3′-F-dTTP are efficient substrates of the RdRp. In the trace in FIG. 21d, a single extension peak indicative of a single-base extension by 3′-N₃-dTMP is seen (6633 Da observed, 6641 Da expected), with no evidence of further incorporation, though the incorporation efficiency was lower than for 2′-F,Me-UTP and 3′-F-dTTP; further optimization may be required. Finally, in the trace in FIG. 21c, a peak indicative of the molecular weight of a primer extension product formed by incorporating 2 Us and 1 TFV (an A analogue) is found (7198 Da observed, 7193 Da expected), in addition to other peaks representing partial incorporation (one U, 6623 Da observed, 6618 Da expected) or misincorporation (3 Us, 7235 Da observed, 7230 Da expected). Importantly, once the TFV was incorporated, there was no further extension, indicating it was an obligate terminator for the RdRp. The result of an additional experiment with TFV-DP is shown in FIG. 22a, in which a longer RNA primer was used with the same template RNA, allowing direct incorporation of TFV. Again, only a single TFV was incorporated (7199 Da observed, 7193 Da expected), despite the presence of 3 additional Us in the template.

The results for Car-TP, which is a G analogue, are shown in FIG. 22b. The most prominent extension peak observed indicates extension by UTP, ATP and CTP followed by complete termination with a Car-TP (10436 Da observed, 10438 Da expected). Despite the inclusion of UTP, ATP and CTP in the mixture with Car-TP, no extension past this point was observed, indicating that Car-TP was an obligate terminator of the SARS-CoV-2 RdRp. In addition, some partial extension peaks were seen, e.g., incorporation of one U (6624 Da observed, 6618 Da expected), and extension up to the position just before the first C in the template strand (10128 Da observed, 10129 Da expected). These results are consistent with results obtained using a higher concentration of Car-TP (see Example 5).

MALDI-TOF MS results for extension by the CTP analogues Lam-TP and Ec-TP are shown in FIG. 23a and FIG. 23b, respectively. There was relatively poor incorporation by these nucleotide analogues. With Lam-TP, a small peak was observed at 8844 Da (8837 Da expected) indicating the incorporation of Lam-TP following multiple incorporated Us and As. In addition, partial extension peaks were observed at 6932 Da indicating extension by two Us (6924 Da expected) and at 8553 Da indicating extension by 2 Us, 4 As and 1 U (8546 Da expected). However, the most prominent peak was observed at 9188 Da, indicating misincorporation by a U at the position where the C analogue Lam-TP would be expected to be incorporated followed by incorporation of the subsequent A (9181 Da expected). Similar results were obtained for Ec-TP. Minimal extension by Ec-TP is indicated by the peak at 8862 Da (8855 Da expected), but a partial extension peak indicating incorporation by 2 Us, 4 As and 1 U at 8555 Da (8546 Da expected), and a prominent peak indicating misincorporation by a U at the position where the C analogue Ec-TP should be incorporated and a subsequent A at 9191 Da (9181 Da expected) were also present. These misincorporation results for both Lam-TP and Ec-TP indicate that SARS-CoV-2 RdRp has low fidelity, which is consistent with the known low fidelity of RdRp (Selisko et al 2018).

### Example 5: Evaluation of a Library of Nucleoside Triphosphates As Terminators of the SARS-CoV and SARS-CoV-2 RdRp Reactions

This example concerns the use of base and sugar modified nucleotides and the FDA approved antiviral drugs Carbovir, Ganciclovir, Cidofovir, Entecavir and Stavudine as inhibitors of SARS-CoV and SARS-CoV-2 RdRps. We tested the ability of the activated (triphosphate) forms of these drugs, Carbovir-5′-triphosphate (Car-TP), Ganciclovir-5′-triphosphate (Gan-TP), Cidofovir diphosphate (Cid-DP), Entecavir-5′-triphosphate (Ent-TP) and Stavudine-5′-triphosphate (Sta-TP) to be incorporated by these RdRps to inhibit RNA replication. The chemical structures of these compounds are shown in FIG. 24. In addition, we tested six other nucleoside triphosphate analogues, 2′-O-methyl UTP (2′-O-Me-UTP), 3′-O-methyl UTP (3′-O-Me-UTP), 2′-fluoro-dUTP (2′-F-dUTP), 2′-amino-dUTP (2′-NH₂-dUTP), biotin-16-dUTP (biotin-dUTP) and desthiobiotin-16-UTP (desthio-UTP), whose structures are also included in FIG. 24. In FIGS. 25 and 26, the structures of eight of these active nucleoside triphosphates are shown along with the structures of their prodrug forms. We used the RdRp of SARS-CoV, the causative agent of SARS, referred to as nsp12, and its two protein cofactors, nsp7 and nsp8, which are required for the processive polymerase activity of nsp12, to perform the polymerase reactions. We also used the equivalent nsp12, nsp7 and nsp8 proteins of SARS-CoV-2, responsible for the COVID-19 pandemic for the same purpose.

In contrast to other viruses, the SARS-CoV and SARS-CoV-2 coronaviruses have very large genomes that encode a 3′-5′ exonuclease (nsp14) involved in proofreading (Shannon et al. 2020), the activity of which is enhanced by the cofactor nsp10 (Bouvet et al 2012). This proofreading function increases replication fidelity. Mutations in nsp14 lead to reduced replication fidelity of the viral genome (Eckerle et al. 2010). Interestingly, while the nsp14/nsp10 complex efficiently excises single mismatched nucleotides at the 3′ end of the RNA chain, it is not able to remove longer stretches of unpaired nucleotides or 3′ modified RNA (Bouvet et al. 2012). In order for the nucleotide analogues to be successful inhibitors of these viruses, they need to overcome this proofreading function. The coronavirus exonuclease activity typically requires a 2′-OH group for excising mismatched nucleotides at the 3′ end of the growing RNA strand (Minskaia et al 2006). However, if there is delayed termination and the offending nucleotide analogue is no longer at the 3′ end or if there is a run of 2 or more modified nucleotides in the growing strand, they will be less likely to be removed by the exonuclease (Bouvet et al 2012, Gordon et al 2020a, b). Nearly all of the nucleotide analogues we selected lack the 2′-OH group (including dideoxynucleotides), have modifications that block the 2′-OH group on the sugar, or are acyclic nucleotide derivatives; such nucleotides will be less likely to be substrates of viral exonucleases.

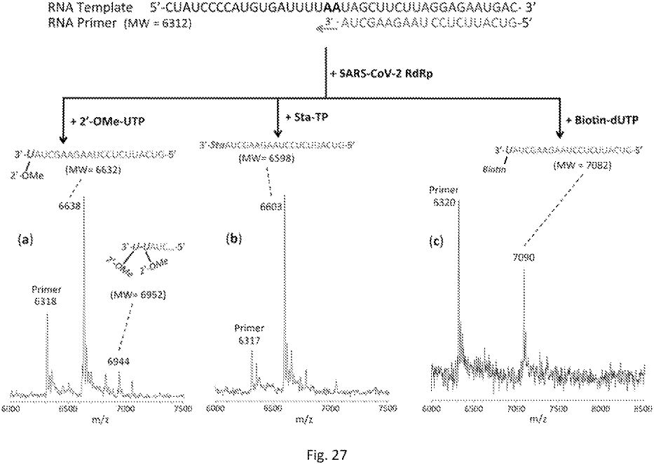

We tested the ability of the active triphosphate forms of the compounds listed above to be incorporated by the RdRps of SARS-CoV or SARS-CoV-2. The RdRp of these coronaviruses, referred to as nsp12, and its two protein cofactors, nsp7 and nsp8, have been shown to be required for the processive polymerase activity of nsp12 in SARS-CoV (Subissi et al. 2014, Kirchdoerfer & Ward 2019). These three components of each coronavirus polymerase complex were cloned and purified as described previously (Kirchdoerfer & Ward 2019; Chien et al. 2020). We then performed polymerase extension assays with 2′-O-methyluridine triphosphate (2′-O-Me-UTP), 3′-O-methyluridine 5′-triphosphate (3′-O-Me-UTP), 2′-fluoro-2′-deoxyuridine triphosphate (2′-F-dUTP), 2′-amino-2′-deoxyuridine triphosphate (2′-NH₂-dUTP), biotin-16-dUTP (Bio-UTP), desthiobiotin-16-UTP (desthio-UTP), Stavudine-TP (Sta-TP), Cidofovir diphosphate (Cid-DP) + UTP + ATP, Carbovir triphosphate (Car-TP) + UTP + ATP + CTP, Ganciclovir 5′-triphosphate (Gan-TP) + UTP + ATP + CTP, or Entecavir triphosphate (Ent-TP) + UTP + ATP + CTP, following the addition of a pre-annealed RNA template and primer to a pre-assembled mixture of the SARS-CoV and/or SARS-CoV-2 RdRp (nsp12) and the two cofactor proteins (nsp7 and nsp8). We also used combinations of nucleotide analogues in some cases to perform the polymerase reaction. The extended primer products from the reaction were analyzed by MALDI-TOF-MS. The sequences of the RNA template and primer used for the polymerase extension assay, which correspond to the 3′ end of the SARS-CoV-2 genome, are indicated at the top of FIGS. 27-37.

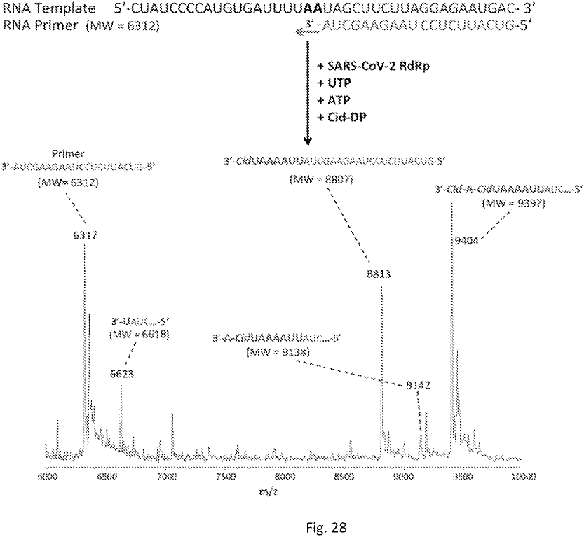

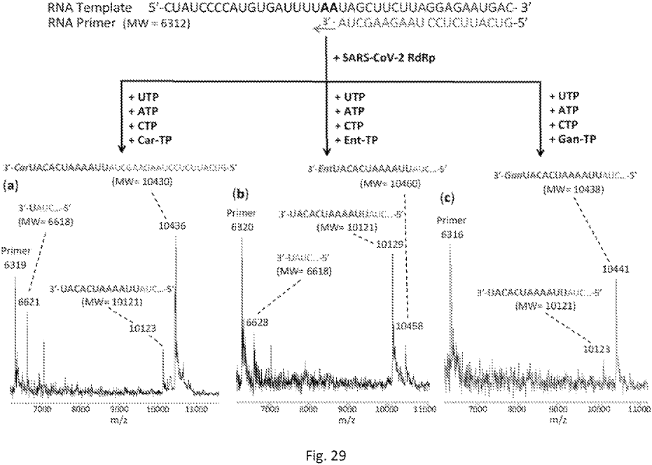

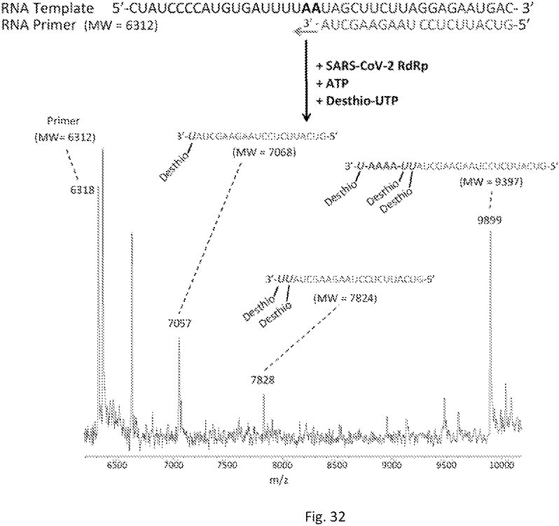

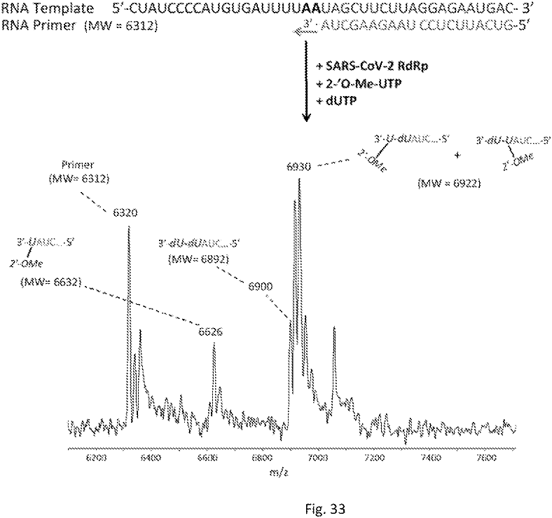

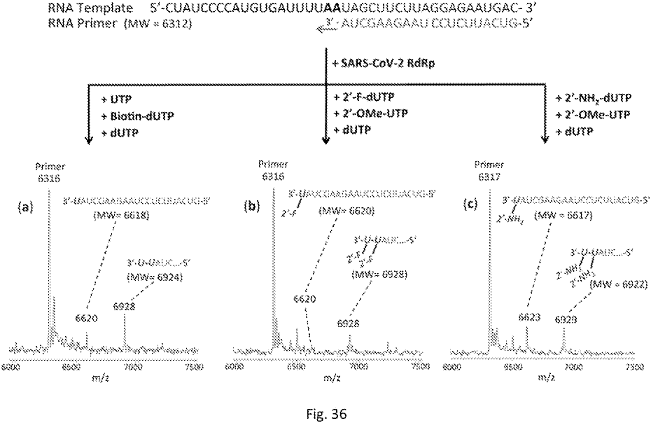

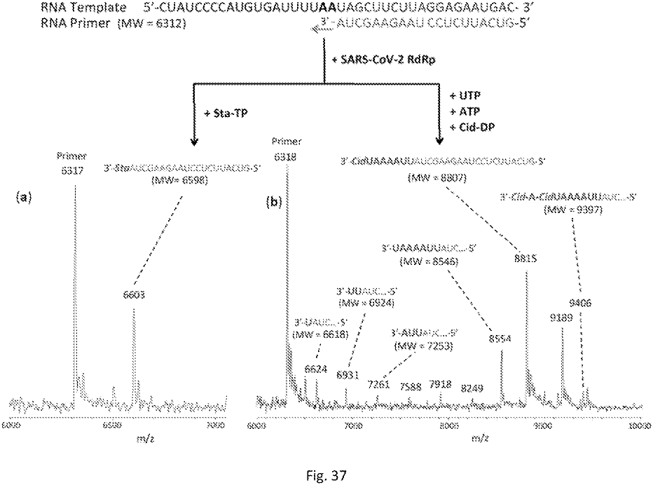

The detailed protocol for the extension reactions are as follows: Extension reactions with SARS-CoV-2 RNA-dependent RNA polymerase: The primer and template (sequences shown in FIGS. 27-37) were annealed by heating to 70° C. for 10 min and cooling to room temperature in 1× reaction buffer. The RNA polymerase mixture consisting of 6 µM nsp12 and 18 µM each of cofactors nsp7 and nsp8 (Chien et al. 2020a, b) was incubated for 15 min at room temperature in a 1:3:3 ratio in 1× reaction buffer. Then 5 µl of the annealed template primer solution containing 2 µM template and 1.7 µM primer in 1× reaction buffer was added to 10 µl of the RNA polymerase mixture and incubated for an additional 10 min at room temperature. Finally 5 µl of a solution containing 2 mM 2′-OMe-UTP (FIG. 27a), 2 mM Sta-TP (FIG. 27b), 2 mM Biotin-dUTP (FIG. 27c), 2 mM Cid-DP + 2 mM UTP + 2 mM ATP (FIG. 28), 2 mM Car-TP + 2 mM UTP + 2 mM ATP + 2 mM CTP (FIG. 29a), 2 mM Ent-TP + 2 mM UTP + 2 mM ATP + 2 mM CTP (FIG. 29b), 2 mM Gan-TP + 2 mM UTP + 2 mM ATP + 2 mM CTP (FIG. 29c), 0.2 mM Sta-TP (FIG. 37a), 0.2 mM Cid-DP + 0.4 mM UTP + 0.4 mM ATP (FIG. 37b), 2 mM desthiobiotin-16-UTP + 2 mM ATP (FIG. 32), 2 mM 2′-OMe-UTP + 2 mM dUTP (FIG. 33), 1 mM UTP, 1 mM Biotin-dUTP and 1 mM dUTP (FIG. 36a), 1 mM 2′-F-dUTP, 1 mM 2′-OMe-UTP and 1 mM dUTP (FIG. 36b), or 1 mM 2′-NH₂-dUTP, 1 mM 2′-OMe-UTP and 1 mM dUTP (FIG. 36c) in 1× reaction buffer was added and incubation was carried out for 2 hrs at 30° C. The final concentrations of reagents in the 20 µl extension reactions were 3 µM nsp12, 9 µM nsp7, 9 µM nsp8, 425 nM RNA primer, 500 nM RNA template, 500 µM 2′-OMe-UTP (FIG. 27a), 500 µM Sta-TP (FIG. 27b), 500 µM Biotin-dUTP (FIG. 27c), 500 µM Cid-DP, 500 µM UTP and 500 µM ATP (FIG. 28), 500 µM Car-TP, 500 µM UTP, 500 µM ATP and 500 µM CTP (FIG. 29a), 500 µM Ent-TP + 500 µM UTP + 500 µM ATP + 500 µM CTP (FIG. 29b), 500 µM Gan-TP, 500 µM UTP, 500 µM ATP and 500 µM CTP (FIG. 29c), 50 µM Sta-TP (FIG. 37a), 50 µM Cid-DP + 100 µM UTP + 100 µM ATP (FIG. 37b), 500 µM desthiobiotin-16-UTP + 500 µM ATP (FIG. 32), 500 µM 2′-OMe-UTP + 500 µM dUTP (FIG. 33), 250 µM UTP, 250 µM Biotin-dUTP and 250 µM dUTP (FIG. 36a), 250 µM 2′-F-dUTP, 250 µM 2′-OMe-UTP and 250 µM dUTP (FIG. 36b), and 250 µM 2′-NH₂-dUTP, 250 µM 2′-OMe-UTP and 250 µM dUTP (FIG. 36c). The 1× reaction buffer contains the following reagents: 10 mM Tris-HCl pH 8, 10 mM KCl, 2 mM MgCl₂ and 1 mM β-mercaptoethanol. Following desalting using an Oligo Clean & Concentrator (Zymo Research), the samples were subjected to MALDI-TOF-MS (Bruker ultrafleXtreme) analysis.

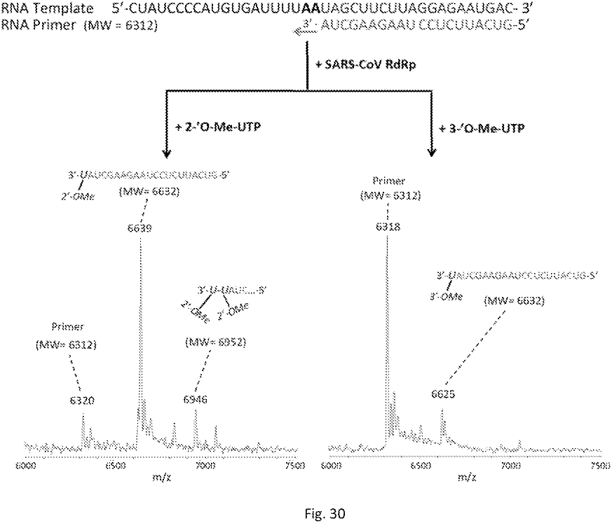

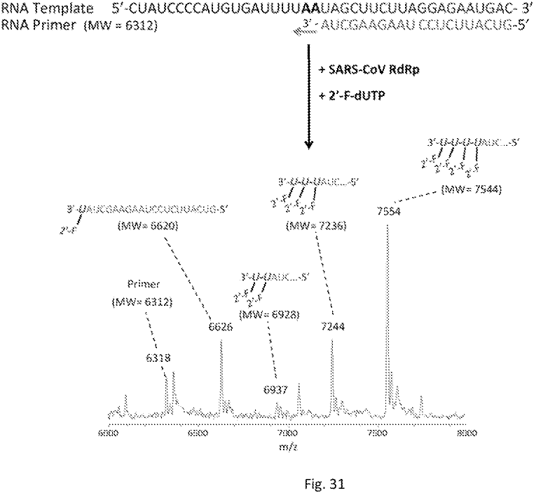

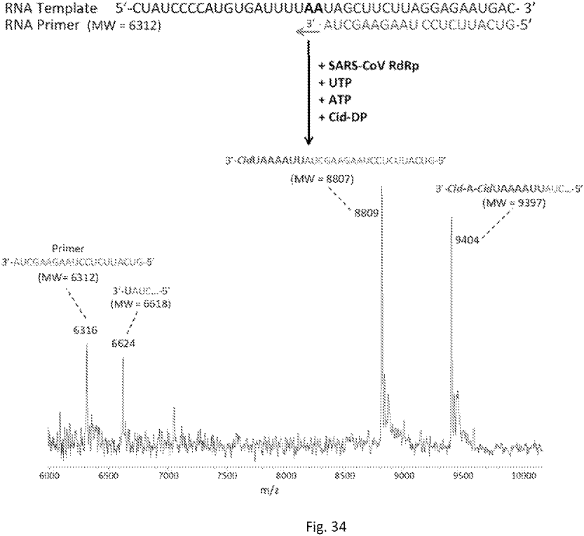

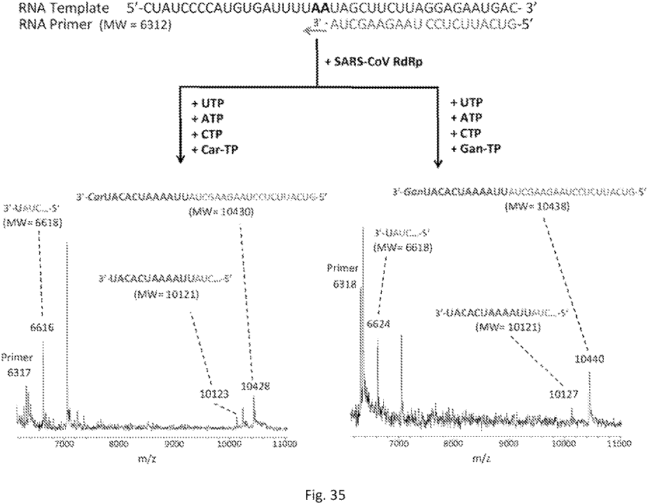

Extension reactions with SARS-CoV RNA-dependent RNA polymerase: The primer and template above were annealed by heating to 70° C. for 10 min and cooling to room temperature in 1× reaction buffer (described above). The RNA polymerase mixture consisting of 6 µM nsp12 and 18 µM each of cofactors nsp7 and nsp8 (Kirchdoerfer and Ward 2019) was incubated for 15 min at room temperature in a 1:3:3 ratio in 1× reaction buffer. Then 5 µl of the annealed template primer solution containing 2 µM template and 1.7 µM primer in 1× reaction buffer was added to 10 µl of the RNA polymerase mixture and incubated for an additional 10 min at room temperature. Finally 5 µl of a solution containing 2 mM Cid-DP + 0.8 mM UTP + 0.8 mM ATP (FIG. 34), 2 mM Car-TP + 0.8 mM UTP + 0.8 mM ATP + 0.8 mM CTP (FIG. 35a), or 2 mM Gan-TP + 0.8 mM UTP + 0.8 mM ATP + 0.8 mM CTP (FIG. 35b), 2 mM 2′-OMe-UTP (FIG. 30a), 2 mM 3′-OMe-UTP (FIG. 30b), or 2 mM 2′-F-dUTP (FIG. 31) in 1× reaction buffer was added and incubation was carried out for 2 hrs at 30° C. The final concentrations of reagents in the 20 µl extension reactions were 3 µM nsp12, 9 µM nsp7, 9 µM nsp8, 425 nM RNA primer, 500 nM RNA template, 500 µM Cid-DP, 200 µM UTP and 200 µM ATP (FIG. 34), 500 µM Car-TP, 200 µM UTP, 200 µM ATP and 200 µM CTP (FIG. 35a), 500 µM Gan-TP, 200 µM UTP, 200 µM ATP and 200 µM CTP (FIG. 35b), 500 µM 2′-OMe-UTP (FIG. 30a), 500 µM 3′-OMe-UTP (FIG. 30b), and 500 µM 2′-F-dUTP (FIG. 31). Following desalting using an Oligo Clean & Concentrator, the samples were subjected to MALDI-TOF-MS analysis.

In the case of the UTP and TTP analogues, because there are two A’s in a row in the next available positions of the template for RNA polymerase extension downstream of the priming site, if they are indeed inhibitors of the polymerase, the extension should stop after incorporating one nucleotide analogue. If they do not serve as terminators, two base extension by the UTP or TTP analogue will be observed. In the case of Cid-DP which is a CTP analogue, UTP and ATP must be provided to allow extension to the point where there is a G in the template strand. If the Cid-DP is then incorporated and acts as a terminator, extension will stop; otherwise, additional incorporation events may be observed. Similarly, for Carbovir-TP, Ganciclovir-TP, and Entecavir-TP, all of which are GTP analogues, UTP, ATP and CTP must be provided to allow extension to the point where there is a C in the template strand. If Car-TP, Gan-TP or Ent-TP is incorporated and acts as a terminator, extension will stop; otherwise, additional incorporation events may be observed. Guided by polymerase extension results we obtained previously for the active triphosphate forms of Sofosbuvir, Alovudine, AZT, Tenofovir-DP and Emtricitabine-TP (Ju et al 2020a, b, Chien et al 2020a, b, Jockusch et al 2020d), various ratios of the nucleotides were chosen in the current work.

The results of the MALDI-TOF MS analysis of the primer extension reactions are shown in FIGS. 27-37. The observed peaks generally fit the nucleotide incorporation patterns described above; however, additional peaks assigned to intermediate stages of the extension reaction and in some cases extension beyond the incorporation of the nucleotide analogue were also observed. We describe the results for the SARS-CoV-2 polymerase in detail; similar results were obtained for the subset of nucleotide analogues tested with the SARS-CoV RdRp.

The results for 2′-OMe-UTP, Sta-TP (which is a T analog) and Biotin-dUTP are presented in FIG. 27. In the case of extension with 2′-OMe-UTP, MS peaks representing incorporation by one 2′-OMe-UTP (6638 Da observed, 6632 Da expected) and to a lesser extent two 2′-OMe-UTPs (6944 Da observed, 6952 Da expected) were observed. Thus, 2′-OMe-UTP shows significant termination after the first incorporation step. This can be a potential drug lead, since 2′-O-methyl modification of RNA occurs naturally and therefore should have relatively low toxicity; in addition, ribose-2′-O-methylated RNA resists the exonuclease (Minskaia et al. 2006). For Sta-TP, a single incorporation peak (6603 Da observed, 6598 Da expected) was seen, indicating that Sta-TP is very efficiently incorporated and achieves complete termination of the polymerase reaction. Since this molecule is a dideoxynucleotide without any hydroxyl groups on the sugar moiety, it may resist exonuclease activity. A 10-fold lower concentration of Sta-TP also resulted in termination of the polymerase reaction (FIG. 37). In the case of Biotin-dUTP, a single incorporation peak was evident (7090 Da observed, 7082 Da expected), suggesting that Biotin-dUTP is also a terminator of the polymerase reaction under these conditions. This indicates that the presence of a modification on the base along with the absence of a 2′-OH group in this nucleotide analogue leads to termination of the polymerase reaction catalyzed by the SARS-CoV-2 RdRp.

The result for the CTP analogue Cid-DP is presented in FIG. 28. Major peaks were observed indicating incorporation of Cid-DP at the 8^{th} position after the initial primer sequence (8813 Da observed, 8807 Da expected) and a further 2 base extension by one ATP followed by one Cid-DP at the 10^{th} position (9404 Da observed, 9397 Da expected). There is no further extension beyond this position, indicative of delayed termination by Cid-DP. A small intermediate peak was also observed indicating extension by the ATP at the 9^{th} position from the initial priming site following the first Cid-DP incorporation (9142 Da observed, 9138 Da expected). A partial UTP extension peak (6623 Da observed, 6618 Da expected) was also observed. A 10-fold lower concentration of Cid-DP also resulted in termination of the polymerase reaction (FIG. 37). An essentially identical result was obtained with the SARS-CoV polymerase (FIG. 34). Delayed termination for Cid-DP has been described for a vaccinia virus DNA polymerase (Magee et al 2005). The investigational drug Remdesivir, which is currently being used for the treatment of COVID-19 under emergency authorization, also displays delayed termination (Gordon et al 2020a, b); this is a major factor in its ability to resist the nsp14 3′-5′ exonuclease activity. Similar resistance to this exonuclease should therefore also occur with Cidofovir due to delayed termination of the polymerase reaction. Thus, Cidofovir and its oral prodrugs are of interest for further investigation to evaluate whether they can evade the viral exonuclease activity. Based on these results, and if potency for viral inhibition in cell culture is demonstrated with limited toxicity, Cidofovir and its related prodrugs may be potential leads for COVID-19 treatment.

The results for the GTP analogues, Car-TP, Ent-TP and Gan-TP are presented in FIG. 29. In each case, extension to the first C position on the template occurs and further extension is blocked in the presence of ATP, UTP and CTP. In more detail, for Car-TP, the major peak observed indicates extension by UTP, ATP and CTP followed by complete termination with a Car-TP (10436 Da observed, 10430 Da expected). In addition, partial extension peaks were seen indicating a single UTP incorporation (6621 Da observed, 6618 Da expected) and extension up to but not including the Car-TP (10123 Da observed, 10121 Da expected). For Ent-TP, a peak was observed indicating extension by UTP, ATP and CTP followed by complete termination by a single Ent-TP (10458 Da observed, 10460 Da expected). Additional peaks are seen representing a single UTP extension (6628 Da observed, 6618 Da expected), and a major peak indicating extension up to but not including the Ent-TP (10129 Da observed, 10121 Da expected). And for Gan-TP, a major peak observed indicated extension by UTP, ATP and CTP followed by complete termination with Gan-TP (10441 Da observed, 10438 Da expected). A small peak representing extension up to but not including Gan-TP (10123 Da observed, 10121 Da expected) was also seen. Similar results were obtained for Car-TP and Gan-TP using the SARS-CoV polymerase (FIG. 35). Both Car-TP and Ent-TP are carbocyclic nucleotides lacking 2′- and 3′-OH groups, while Gan-TP is an acyclic nucleotide lacking a ribose ring. All three are expected to resist exonuclease activity. These results also indicate that Car-TP and Gan-TP are better terminators than Ent-TP, and their prodrugs can be evaluated as therapeutics for COVID-19 and SARS.

FIG. 30 shows a side-by-side comparison of the results with 2′-O-Me-UTP and 3′-O-Me-UTP using the SARS-CoV polymerase. The results for 2′-O-Me-UTP are practically identical to those with SARS-CoV-2 in FIG. 33, indicating that 2′-O-Me-UTP exhibits significant polymerase reaction termination. The 3′-O-Me-UTP results are consistent with its being an obligate terminator, but with lower incorporation efficiency, represented by a small single-incorporation peak (6625 Da observed, 6632 Da expected).

In FIG. 31, the results are shown for incorporation of 2′-F-dUTP by SARS-CoV RdRp. 2′-F-dUTP was incorporated very efficiently, but also was incorporated opposite the UTPs in the template strand. This apparent mismatch incorporation may be due to the relatively low fidelity of SARS-CoV RdRp.

The results for desthio-UTP are presented in FIG. 32. Desthio-UTP incorporation opposite each A in the template is observed, just like a UTP. Thus, this nucleotide is incorporated and does not terminate the polymerase reaction. These results indicate that significant modification on the base of the UTP does not affect its incorporation activity by SARS-CoV-2 RdRp.

FIG. 33 presents the results of an experiment where both 2′-O-Me-UTP and dUTP were added together. The major peak occurred at 6930 Da (6922 Da expected) representing incorporation by both dUTP and 2′-O-Me-UTP in adjacent positions. In addition, some partial extension peaks of a single 2′-O-Me-UTP (6626 Da observed, 6632 Da expected) and two dUTPs (6900 Da observed, 6892 Da expected) were found. The incorporation of a dUTP, a 2′-O-Me-UTP, both of which lack a 2′-OH group, or their combination would enable them to resist the nsp14 3′-5′ exonuclease activity.

FIG. 36 shows three mass spectra of the polymerase reaction products using equimolar combinations of nucleotide analogues, (a) biotin-dUTP, dUTP, and UTP, (b) 2′-F-dUTP, 2′-O-Me-UTP and dUTP, and (c) 2′-NH₂-dUTP, 2′-O-Me-UTP and dUTP, to determine their relative incorporation efficiencies. Based on the results shown in FIG. 36a, biotin-dUTP and dUTP have lower incorporation efficiency than the natural UTP for SARS-CoV-2 RdRp, since peaks are only observed for UTP extension, either one UTP (6620 Da observed, 6618 Da expected) or two UTPs (6928 Da observed, 6924 Da expected). In FIG. 36b, it is seen that 2′-F-dUTP is incorporated far better than 2′-O-Me-UTP and dUTP, with the only evident peaks in the spectrum at 6620 Da (6620 Da expected) for extension by one 2′-F-dUTP, and at 6928 Da (6928 Da expected) for extension by two 2′-F-dUTPs. Finally, as shown in FIG. 36c , 2′-NH₂-dUTP is more efficiently incorporated than 2′-O-Me-UTP and dUTP as revealed by the presence of evident peaks only at 6623 Da (6617 Da expected) for extension by one 2′-NH₂-dUTP and at 6929 Da (6922 Da expected) for extension by two 2′-NH₂-dUTPs. The results indicate that 2′-F-dUTP and 2′-NH₂-dUTP behave like UTP, and are not terminators of the polymerase reaction.

In summary, these results demonstrate that the library of nucleotide analogues we tested could be incorporated by the RdRps of SARS-CoV-2 and SARS-CoV. Of the 11 tested, 6 exhibited complete termination of the polymerase reaction (3′-OMe-UTP, Car-TP, Gan-TP, Sta-TP, Ent-TP, Biotin-dUTP), 2 showed incomplete or delayed termination (Cid-DP, 2′-OMe-UTP), and 3 did not terminate the polymerase reaction (2′-F-dUTP, 2′-NH₂-dUTP and desthiobiotin-16-UTP) using the RdRp of SARS-CoV and/or SARS-CoV-2. Their prodrug versions (FIGS. 25 and 26) are available or can be readily synthesized using the ProTide approach (Alanazi et al. 2019). The ProTide approach was used very successfully to develop Sofosbuvir and Remdesevir for treatment of HCV and COVID-19, respectively. It may be advantageous to use ProTide prodrug forms containing a phosphate masked by a hydrophobic phosphoramidate group for the five drugs whose structures are shown in FIG. 26, because such prodrugs can be delivered into cells and converted to the triphosphate more rapidly, and potentially improve the bioavailability and potency of these compounds. The five drugs (Ganciclovir/Valganciclovir, Cidofovir, Abacavir, Stavudine and Entecavir (FIG. 26)) are FDA-approved medications for treatment of other viral infections and their toxicity profile is well established, while Brincidofovir is an experimental oral antiviral drug. Thus, our results provide a molecular basis for further evaluation of these prodrugs in SARS-CoV-2 virus inhibition and animal models to test their efficacy for the development of potential COVID-19 therapeutics.

### Example 6: SARS-CoV-2 Exonuclease Resistance of RNAs Terminated By a Library of Nucleotide Analogues

We tested a library of nucleotide analogues for their ability to be incorporated by and terminate extension by SARS-CoV and SARS-CoV-2 RdRp (Jockusch et al 2020b). Many of the compounds tested showed immediate or delayed termination. We also examined whether RNA extended with these compounds showed resistance to excision by SARS-CoV-2 exonuclease (nsp14) in the presence of the nsp14 accessory protein nsp10. Our study indicated, for instance, that Sofosbuvir had higher relative resistance to exonuclease than either Remdesivir or UMP (see Example 7 below, Jockusch et al 2020c) .

Here we examined a library of additional modifications at the 3′ end of the RNA for their ability to inhibit exonuclease activity, including CMP, 2′-O-Me-CMP, 2′-F-dCMP, Stavudine-MP, Tenofovir, AZT-MP, Biotin-16-dUMP, Carbovir-MP and Ganciclovir-MP. RNAs modified with CMP, 2′-O-Me-CMP and 2′-F-dCMP at the 3′ end of the RNA primer-loop templates were purchased from a commercial supplier. A different set of template-loop-primers were extended at the 3′ end with Stavudine-MP, Tenofovir, AZT-MP, Biotin-dUMP, Carbovir-MP and Ganciclovir-MP using HIV-RT or SuperScript IV reverse transcriptase to generate the corresponding 3′ end modified RNAs. Following purification, the resulting extended oligonucleotides were treated with exonuclease nsp14/nsp10 and the purified cleavage products were examined by MALDI-TOF-MS to assess their resistance to exonuclease cleavage.

The detailed procedure for generation of polymerase extended RNA and the subsequent exonuclease reactions in the presence of inhibitors is as follows:

Reagents: HIV reverse transcriptase was purchased from Millipore Sigma (St. Louis, MO) and SuperScript IV reverse transcriptase was purchased from Thermo Fisher (Life Technologies, Grand Island, NY). The 3′-exonuclease, referred to as nsp14, and its protein cofactor, nsp10, were purchased from LSBio (Seattle, WA) . Nucleoside triphosphates and nucleoside triphosphate analogues were purchased from TriLink BioTechnologies (Biotin-16-dUTP, Ganciclovir-TP, AZT-TP), Santa Cruz Biotechnology (Stavudine-TP, Carbovir-TP) and Alfa Chemistry (Tenofovir-DP). The RNA oligonucleotides (template-loop-primers) were purchased from Dharmacon (Horizon Discovery, Lafayette, CO).

Synthesis of nucleotide analogue extended RNAs using reverse transcriptase: The RNA template-loop-primers (5′-UUUUCUACGCGUAGUUUUCUACGCG-3′ for biotin-dUTP, Stavudine-TP or AZT-TP extension reactions; 5′-UUUUCUCCGCGUAGUUUUCUACGCG-3′ for Carbovir-TP or Ganciclovir-TP extension reactions; 5′-UUUUCUUCGCGUAGUUUUCUACGCG-3′ for Tenofovir-DP extension reactions) were annealed by heating to 75° C. for 3 min and cooling to room temperature in 1× HIV RT or 1× SuperScript IV reaction buffer. The reverse transcriptase mixture consisting of 27 U of HIV RT or 200 U of SuperScript IV RT in the appropriate 1× buffer. Then 10 µL of the appropriate annealed RNA template-loop-primer solution (10 µM) in 1× reaction buffer was added to 8 µL of the RNA polymerase mixture and incubated for 15 min at room temperature. Finally, 2 µL of a solution containing 5 mM Biotin-dUTP, 10 mM Stavudine-TP, 10 mM Carbovir-TP, 10 mM Tenofovir-DP, 10 mM AZT-TP or 10 mM Ganciclovir-TP in 1× reaction buffer was added and incubation was carried out for 2-3 hr at 45° C. The 20 µL extension reactions contained 27 U HIV-RT or 200 U SuperScript IV RT, 2.5 µM RNA template-loop-primer, and 500 µM Biotin-dUTP, 1 mM Stavudine-TP, 1 mM Carbovir-TP, 1 mM Tenfovir-DP, 1 mM AZT-TP or 1 mM Ganciclovir-TP. The 1× reaction buffer for the HIV RT contains the following reagents: 10 mM Tris-HCl pH 8, 10 mM KCl, 2 mM MgCl₂ and 1 mM β-mercaptoethanol. The buffer for SuperScript IV RT is proprietary. Desalting of the reaction mixture was performed with an Oligo Clean & Concentrator kit (Zymo Research) resulting in ~10 µL purified aqueous RNA solutions. 2 µL of each solution were subjected to MALDI-TOF-MS (Bruker ultrafleXtreme) analysis. The remaining ~8 µL extended template-loop-primer solutions were used to test exonuclease activity as described below.

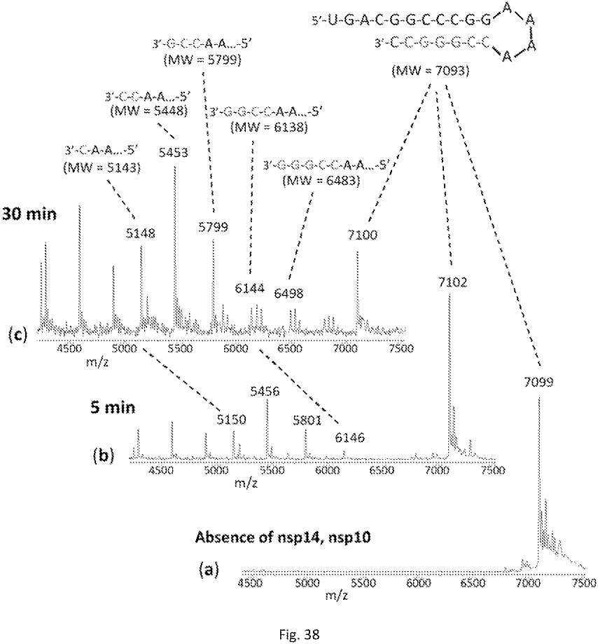

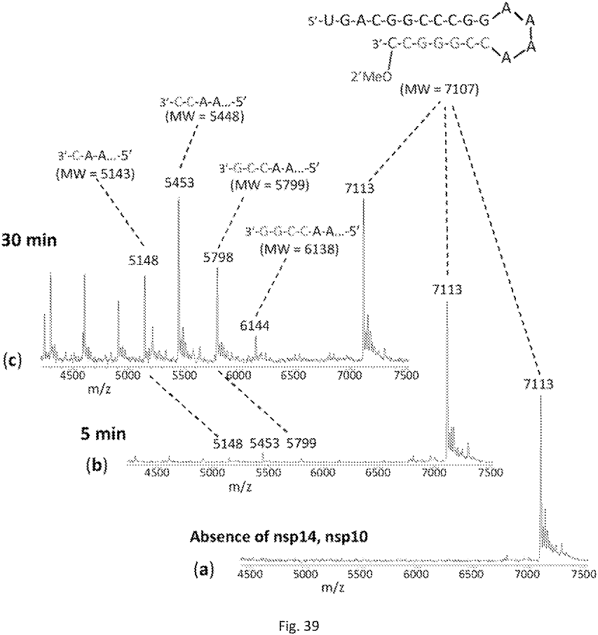

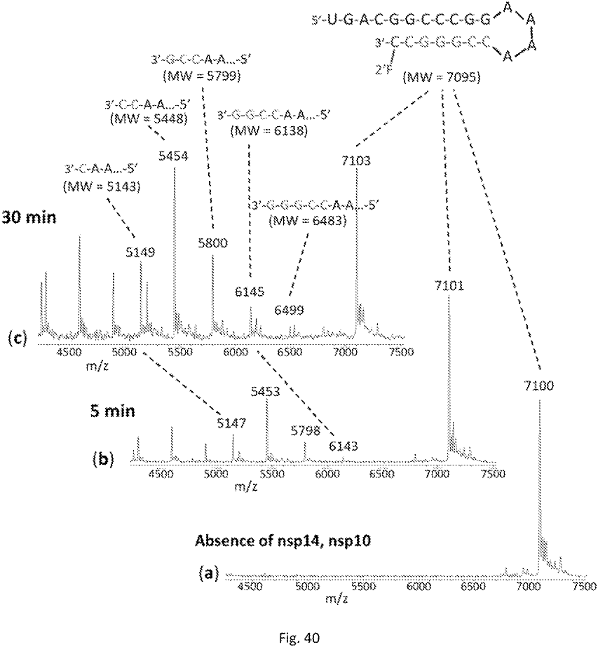

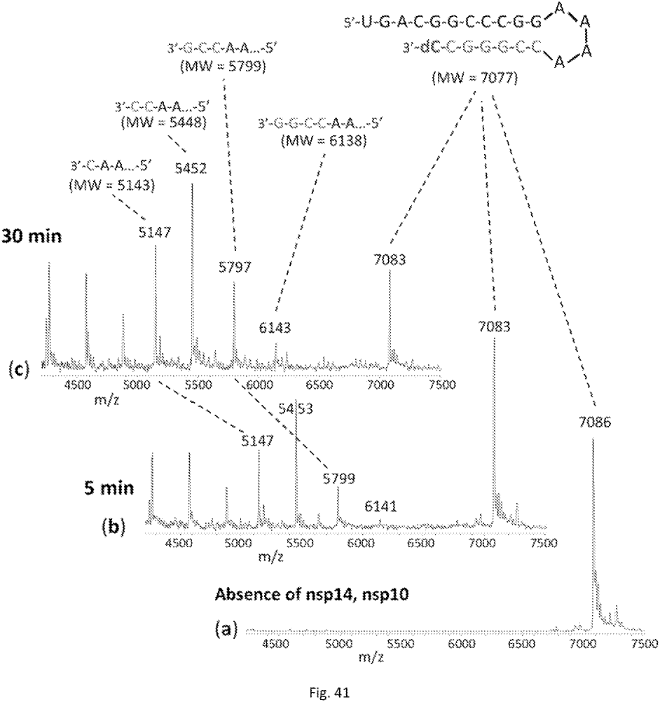

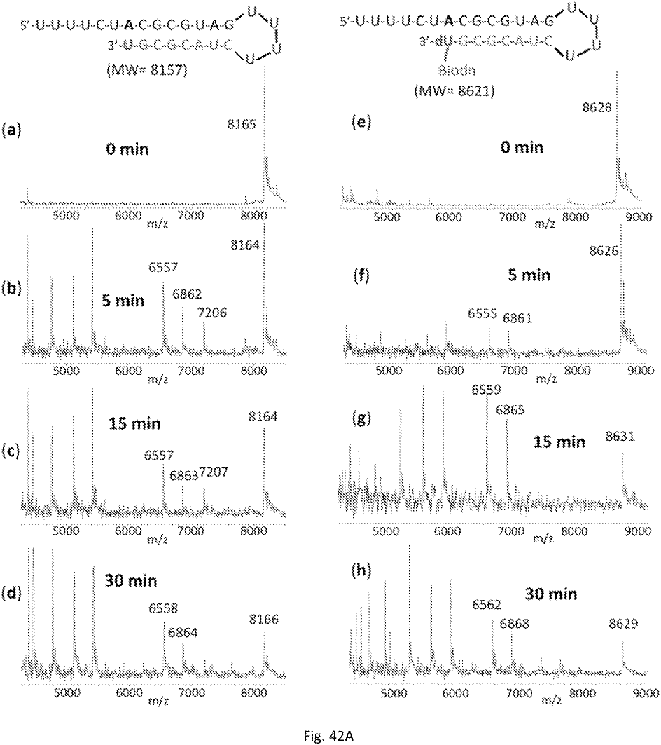

Exonuclease reactions with SARS-CoV-2 nsp14/nsp10 complex: The synthetic RNA template-loop-primers with C, 2′-OMe-C, 2′-F-dC or dC or U at the 3′ terminus (sequences shown in FIGS. 38-41, 42A(a)-(d) respectively) and the reverse transcriptase-extended template-loop-primers with Biotin-dUMP (FIG. 42A(e)-(h)), Stavudine-MP (FIG. 42B(i)-(l)), Carbovir-MP (FIG. 42B(m)-(p)), Tenofovir, AZT-MP or Ganciclovir-MP at the 3′ end, obtained as described in the previous paragraph, were annealed by heating to 75° C. for 3 min and cooling to room temperature in 1× exonuclease reaction buffer. The exonuclease nsp14 (500 nM) and its protein cofactor, nsp10 (2 µM), were incubated for 15 min at room temperature in a 1:4 ratio in 1× exonuclease reaction buffer. Then 10 µL of the annealed extended RNA template-loop-primer solution (2 - 3.2 µM) in 1× exonuclease reaction buffer was added to 10 µL of the exonuclease mixture and incubated at 37° C. for 5, 15 or 30 min. The reactions were stopped by addition of 2.2 µL of EDTA solution (100 mM). The final concentrations of reagents in the 20 µL reactions were 250 nM nsp14, 1 µM nsp10 and 1 - 1.6 µM extended RNA template-loop-primer. The 1× exonuclease reaction buffer contains the following reagents: 40 mM Tris-HCl pH 8, 1.5 mM MgCl₂, 50 µM ZnCl₂ and 5 mM DTT. Following desalting using an Oligo Clean & Concentrator (Zymo Research), the samples were subjected to MALDI-TOF-MS (Bruker ultrafleXtreme) analysis.

The preliminary data presented in this example are described as follows. The results for synthetic templates with C or modified C (2′-O-Me-CTP, 2′-F-dCMP, dCTP) at the 3′ terminus are presented in FIGS. 38-41. In each case, treatment with the exonuclease complex (nspl4, nsp10) was carried out for 5 or 30 minutes at 37° C. At 5 minutes, it is clear that there is much more full-length RNA remaining when there is 2′-O-Me-CTP at the 3′ terminus than for RNAs with C, dC or 2′-F-dCMP at the 3′ terminus. The peaks whose molecular weight are shown are those that can be accounted for by cleavage from the 3′ end. Lower molecular weight peaks on the far left in each mass spectrum could be due to endonucleolytic cleavage which has been reported for the SARS-CoV-2 exonuclease in solution tests (Baddock et al 2020).

The results for UMP, Biotin-dUMP, Stavudine-MP and Carbovir-MP extended RNA are presented in FIGS. 42A and 42B. Reactions were carried out in the absence of the exonuclease complex (0 min) and in the presence of the exonuclease complex for 5, 15 and 30 minutes. The results are presented for UMP in FIG. 42A(a)-(d) (0-30 min, respectively), for Biotin-dUMP in FIG. 42A(e)-(h) (0-30 min), for Stavudine-MP in FIG. 42B(i)-(l) (0-30 min) and for Carbovir-MP in FIG. 42B(m)-(p) (0-30 min). Among these, the slowest cleavage by exonuclease was obtained when Biotin-dUMP or Carbovir-MP were present at the 3′ terminus of the RNA, seen most clearly by comparing the results at the 5 minute time point (FIG. 42A(b), FIG. 42A(f), FIG. 42B(j), and FIG. 42B(n)). Again, the peaks whose molecular weights are shown are those that can be accounted for by cleavage from the 3′ end.

In summary, based on the results in FIGS. 38-41, it was determined that 2′-O-methyl CMP at the 3′ terminus is resistant to excision by the SARS-CoV-2 exonuclease relative to CMP, dCMP and 2′-F-dCMP. Similarly, based on the results in FIGS. 42A and 42B, modification at the 3′ end with Stavudine-MP, Biotin-dUMP and Carbovir-MP resulted in 50-75% full length RNA remaining at 5 minutes and 20-50% full length RNA remaining at 15 minutes, with the most protection from cleavage at 5 minutes occurring for Biotin-16-dUMP and Carbovir-MP.

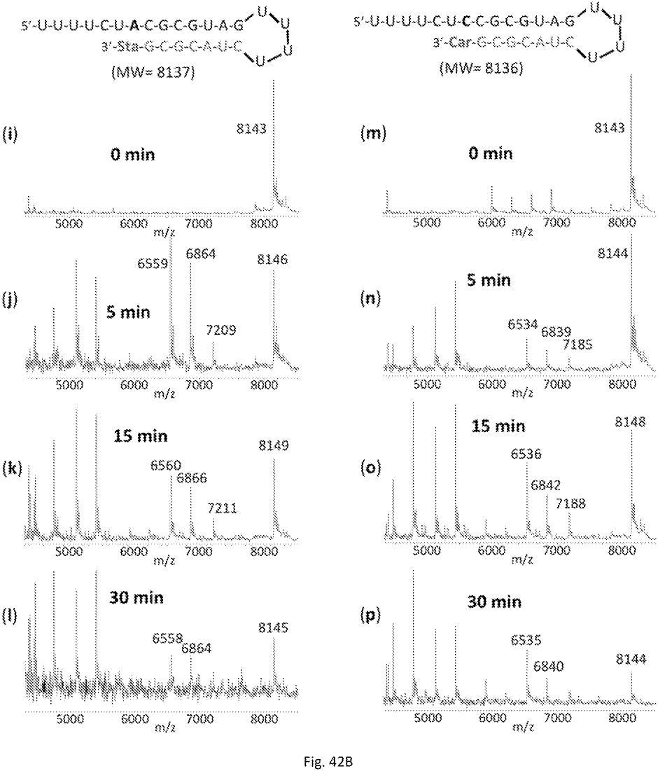

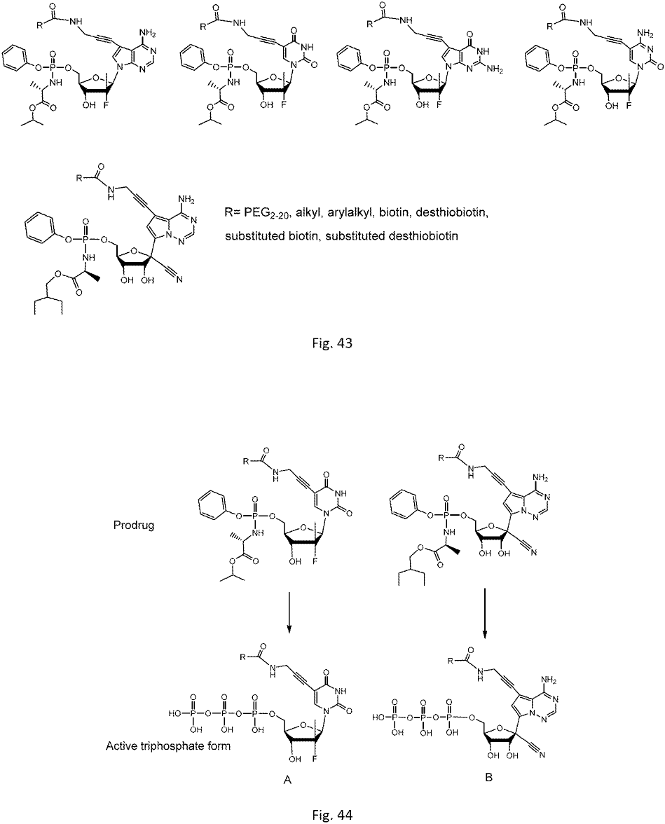

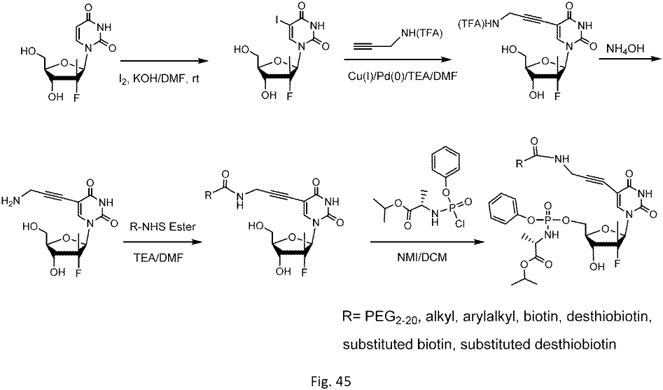

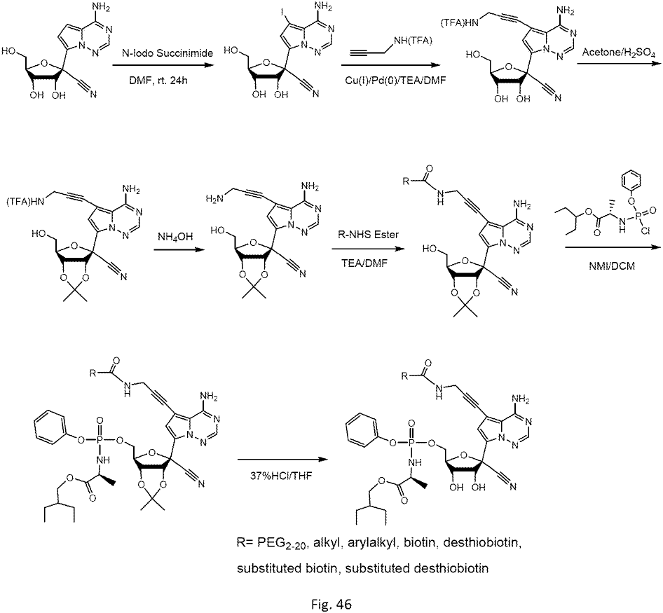

It is clear from these results that the C5-modified nucleotide analogue, Biotin-16-dUTP, after incorporation as Biotin-16-dUMP, shows more resistance to exonuclease cleavage than nucleotides without base modifications, indicating that modifications on the base can contribute to its ability to resist cleavage by the exonuclease. Therefore, placement of modifications on the C5-position of pyrimidines or the C7-position of deazapurines in some of the existing antiviral nucleotide analogues would offer more protection from exonuclease cleavage once they are incorporated into the 3′ end of viral RNAs. In particular for drugs that show less resistance to exonuclease such as Remdesivir, this modification should increase their resistance to exonuclease excision and their overall efficacy. The same modification on Sofosbuvir would make it even more resistant to excision by the SARS-CoV-2 exonuclease. Modifications at the C5 position of pyrimidines and the C7-position of deazapurines will still allow the modified nucleotides to be incorporated by polymerases (Ju et al 2006). We designed such modified nucleotide analog prodrugs; example structures are shown in FIG. 43. The mechanism of their conversion to activated forms inside cells is presented in FIG. 44. Example synthetic schemes for their synthesis are presented in FIGS. 45 and 46.

Starting from the nucleoside (2′R)-2′-deoxy-2′-fluoro-2′-methyluridine (FIG. 45), iodination is carried out to afford (2′R)-2′-deoxy-2′-fluoro-2′-methyl-5-I-uridine, which can be further alkylated by Sonogashira coupling using a TFA protected propargyl amine (Anilkumar et al 2015). After deprotection of the TFA group, the resulting free amino compound can be coupled with a variety of reactive R substituted-NHS esters yielding a C5-substituted nucleoside, which can be further converted to the active nucleotide phosphoramidate prodrug by treatment with freshly prepared chlorophosphoramidate reagent in the presence of N-methyl imidazole (Ross et al 2011, Sofia et al 2010).

Using 1′-cyano-substituted 4-aza-7,9-dideazaadenosine C-nucleosides as starting material (FIG. 46) (Cho et al 2012), iodination is carried out to afford a 7-iodo nucleoside (Sachin et al 2016), which can be further alkylated by Sonogashira coupling using a TFA protected propargyl amine. Then 2′,3′ hydroxyl groups will be protected with acetal and the TFA protective group can be removed to give a free amino compound for coupling with a variety of reactive R substituted-NHS esters yielding a C7-substituted-nucleoside. The nucleotide phosphoramidate prodrug can be synthesized by treatment with freshly prepared chlorophosphoramidate reagent in the presence of N-methyl imidazole (Ross et al 2011, Sofia et al 2010). Final deprotection of acetal will yield 1′-cyano-4-aza-7,9-dideaza-C7-substituted-adenosine C-nucleotide phosphoramidate (Siegel et al 2017).

### Example 7: Sofosbuvir Extended RNA Is More Resistant to the SARS-CoV-2 Proofreading 3′-5′ Exonuclease than Remdesivir Extended RNA

Comparison of the structure-activity relationships of Sofosbuvir and Remdesivir. Sofosbuvir (FIG. 4a), a pyrimidine nucleotide analogue prodrug, has a hydrophobic masked phosphate group that enhances its ability to enter host cells. The prodrug is subsequently converted into an active triphosphate form (SOF-TP; 2′-F,Me-UTP) by cellular enzymes, enabling it to inhibit the HCV RdRp NS5B2 (Eltahla et al 2015, Sofia et al 2010). SOF-TP is incorporated into RNA by the viral RdRp, and due to the presence of fluoro and methyl modifications at the 2′ position, inhibits further RNA chain extension, which halts RNA replication and viral multiplication. A related purine nucleotide ProTide (Alanazi et al 2019) prodrug, Remdesivir (FIG. 4b), originally developed for the treatment of Ebola virus infections, though not successfully, is being used for COVID-19 treatment. Unlike Sofosbuvir (FIG. 4a), Remdesivir has both 2′- and 3′-OH groups (FIG. 4b), and a cyano group at the 1′ position which is responsible for RdRp inhibition.

Analyzing the structures of the active triphosphate forms of Sofosbuvir (FIG. 4a) and Remdesivir (FIG. 4b), both of which have been shown to inhibit the replication of specific RNA viruses (Sofosbuvir for HCV, Remdesivir for SARS-CoV-2), we noted that the 2′-modifications in Sofosbuvir (the fluoro and methyl groups) are smaller than the 1′-cyano group and the 2′-OH group in Remdesivir. The bulky cyano group in close proximity to the 2′-OH may produce steric hindrance, thereby impacting the polymerase reaction termination efficiency of the activated form of Remdesivir. It was recently reported that, using the MERS-CoV and SARS-CoV-2 RdRps, RDV-TP had higher incorporation efficiency than ATP (Gordon 2020a, 2020b). However, RDV-TP does not cause immediate polymerase reaction termination; rather, it leads to delayed polymerase termination, likely due to its 1′-cyano group and free 2′-OH and 3′-OH groups.

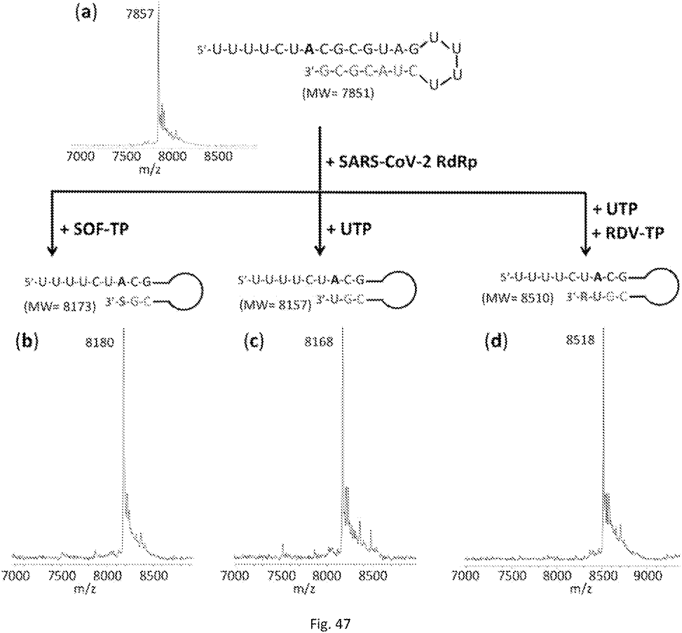

SARS-CoV-2 exonuclease resistance study of RNAs terminated by the triphosphates of Sofosbuvir and Remdesivir. To demonstrate whether the RNA terminated by the triphosphate forms of Sofosbuvir (SOF-TP) and Remdesivir (RDV-TP) have the potential to resist the SARS-CoV-2 proofreading activity, we carried out polymerase extension reactions followed by exonuclease digestion reactions. First, using the replication complex assembled from SARS-CoV-2 nsp12 (the viral RdRp) and nsp7 and nsp8 proteins (RdRp cofactors), the nucleotide analogues were incorporated at the 3′ end of the double-stranded segment of the RNA template-loop-primer shown at the top of FIG. 47. Because the template strand consists of an A at the next position, SOF-TP (a UTP analogue) or UTP are incorporated in a single-nucleotide extension reaction. On the other hand, because the next base in the template strand after the A is a U, in order to incorporate the ATP analogue RDV-TP, a two-nucleotide extension (UTP followed by RDV-TP) occurs. After purification to remove the RdRp and nucleoside triphosphates, the extended RNA is treated with the SARS-CoV-2 exonuclease complex consisting of SARS-CoV-2 nsp14 (the viral exonuclease) and nsp10 (an exonuclease cofactor) to determine whether excision of the incorporated nucleotide analogues takes place. Our prediction was that SOF would be at least partially protected from the exonuclease due to the presence of 2′-fluoro and 2′-methyl groups in place of the 2′-OH group, but that RDV and UMP, both of which have a 2′-OH and 3′-OH, would be less protected. This was indeed what we observed as described below.

We performed polymerase extension reactions with SOF-TP, UTP, and RDV-TP + UTP, following the addition of the pre-annealed RNA template-loop-primer to a pre-assembled mixture of nsp12, nsp7 and nsp8. The extended RNA products from the reaction were subjected to MALDI-TOF-MS analysis to confirm that the expected RNA products were formed. The sequence of the RNA template-loop-primer used for the polymerase extension assay, which has previously been described (Hillen et al 2020), is shown at the top of FIG. 47.

The MALDI-TOF mass spectrum of the unextended RNA template-loop-primer is shown in FIG. 47a. As shown in FIG. 47b, following incubation with the SARS-CoV-2 replication complex, complete conversion of the RNA template-loop-primer (7851 Da expected) to the Sofosbuvir-terminated extension product was observed (8180 Da observed, 8173 Da expected). Similarly, as shown in FIGS. 47c and 47d, quantitative extension was seen with UTP (8168 Da observed, 8157 Da expected) and by UMP and RDV (8518 Da observed, 8510 Da expected), respectively.

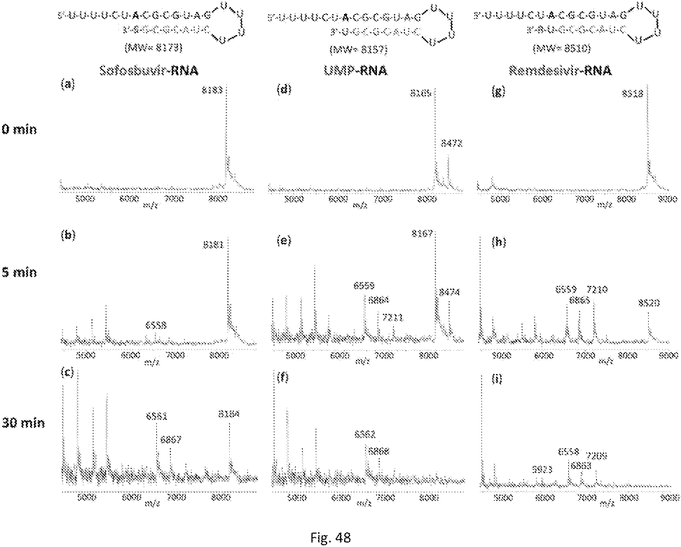

The above RNA extension products were purified and then incubated with the exonuclease complex (nsp14 and nsp10); the results are presented in FIG. 48. The MS trace for the Sofosbuvir extension product (Sofosbuvir-RNA) in the absence of exonuclease (0 min) is shown in FIG. 48a. Only the expected peak at 8183 Da (8173 Da expected) is observed. After 5 min treatment with the nsp14/nsp10 exonuclease complex (FIG. 48b), there is minimal appearance of cleavage products (e.g., the small 6558 Da peak representing cleavage of 5 nucleotides). Even after 30 min exonuclease treatment (FIG. 48c), there is a significant amount of the intact Sofosbuvir-RNA remaining, and the appearance of lower molecular weight peaks, for example at 6867 Da and 6561 Da (removal of 4 and 5 nucleotides from the 3′ end respectively). The MS result for the purified RNA extended with UMP (UMP-RNA) is shown in FIG. 48d (8165 Da observed, 8157 expected). In addition to the expected extension peak, a smaller peak at 8472 represents mismatch incorporation of an additional U; this is likely due to the high concentration of UTP used and the relatively low fidelity of the RdRp (Chien et al 2020). After 5 min treatment with the exonuclease complex (FIG. 48e), there is substantial cleavage as indicated by the peaks at 7211 Da, 6864 Da and 6559 Da (3, 4 and 5 nucleotide cleavage products, respectively). At 30 min (FIG. 48f), the extended RNA product peak completely disappears with the presence of only cleavage fragment peaks. Finally, for the UMP + RDV extended RNA (Remdesivir-RNA), shown in FIG. 48g, the RNA extension product peak is substantially reduced after 5 min exonuclease treatment (FIG. 48h), with conversion to smaller fragments, e.g., 7210 Da, 6865 Da and 6559 Da (removal of 3, 4 or 5 nucleotides from the 3′ end). At 30 min (FIG. 48i), there is no visible Remdesivir-RNA peak remaining, with only cleavage product peaks observed, e.g., at 7209 Da, 6863 Da, 6558 Da and 5923 Da (cleavage of 4, 5, 6 and 8 nucleotides respectively). Clearly, by comparing the results in FIG. 48b with FIG. 48h, and FIG. 48c with FIG. 48i, there is substantially more exonuclease cleavage of Remdesivir-RNA than Sofosbuvir-RNA, and from the results in FIGS. 48d-i, it is also apparent that Remdesivir-RNA is cleaved by the SARS-CoV-2 exonuclease more rapidly than RNA extended with UMP.

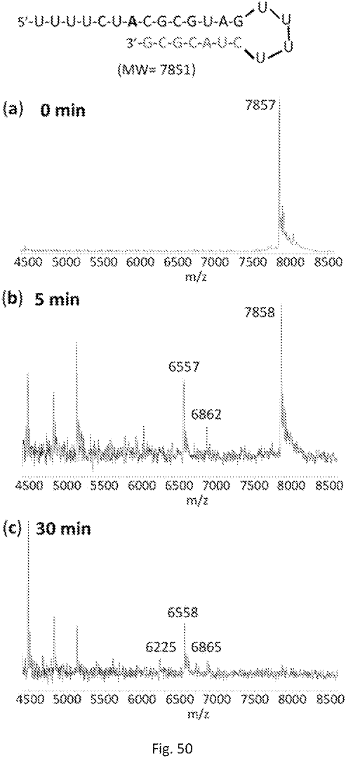

As a control, exonuclease cleavage results for the unextended RNA template-loop-primer that is used to generate the extended RNA products shown in FIG. 47, are presented in FIG. 50. A similar cleavage pattern was observed as in FIG. 48 for natural nucleotide (UMP) or nucleotide analogue (SOF and RDV) extended RNAs. In either case (extended or unextended RNA), the cleavage products with the highest molecular weights observed by MALDI-TOF MS indicated removal of 3 or 4 nucleotides. Such rapid cleavage of RNA products by the SARS-CoV exonuclease complex (nsp14/nsp10) has been observed previously (Bouvet et al 2012, Ferron et al 2018).

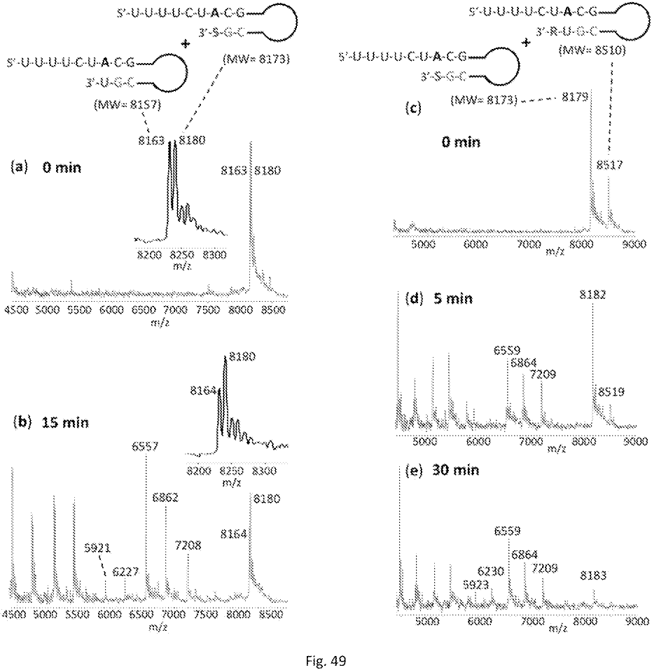

In order to further compare the relative nucleotide excision among the different extended RNAs, in FIG. 49, similar experiments were carried out as in FIG. 48, but the exonuclease treated SFV and UMP extended RNA products (FIGS. 49a-b) were combined for purification, followed by MALDI-TOF-MS analysis. The MS spectrum for the untreated RNA products is shown in FIG. 49a. In the inset it is possible to differentiate the RNA extended with UMP (8163 Da observed, 8157 Da expected) and SFV (8180 Da observed, 8173 Da expected). The MS spectrum for the 15 min exonuclease-treated RNA products is shown in FIG. 49b. It is clear in the inset that the RNA peak representing UMP extension (8164 Da) is reduced to a substantially greater extent than the peak representing the SFV extended RNA (8180 Da). Similarly, the exonuclease treated SFV and UMP+RDV extended RNA products (FIGS. 49c-e) were combined for purification, followed by MALDI-TOF-MS analysis. The MS spectrum for the untreated RNA products is shown in FIG. 49c. The peak at 8179 Da represents the SFV extended product and the peak at 8517 Da represents the UMP+RDV extended product. The MS spectrum for the 5 min exonuclease-treated RNA products is shown in FIG. 49d. There is some reduction in the height of the SFV-extended RNA peak, and a much more substantial reduction in the height of the UMP+RDV extended RNA peak. The peaks at 7209 Da, 6864 Da and 6559 Da represent RNA fragments after cleavage of 3-5 nucleotides, presumably mainly from the UMP+RDV extended RNA. In the case of the 30 min exonuclease-treated RNA products (FIG. 49e), there is still some SFV extended RNA remaining (8183 Da) while the UMP+RDV extended RNA peak is at baseline level. This experiment confirms the results in FIG. 48, that Sofosbuvir is more protected from cleavage by the SARS-CoV-2 exonuclease than UMP or Remdesivir.

### Example 8: HCV NS5A Inhibitors Daclatasvir and Velpatasvir Inhibit SARS-CoV-2 Polymerase Reaction

Structures of NS5A inhibitors are included in FIG. 51A. In this example, we investigated whether the NS5A inhibitors Daclatasvir and Velpatasvir inhibited the reaction catalyzed by the SARS-CoV-2 RdRp complex.

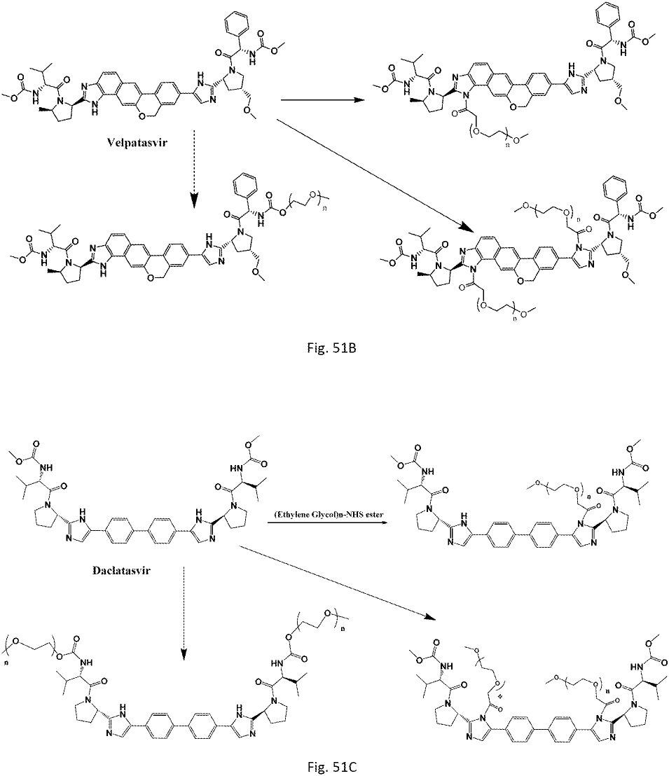

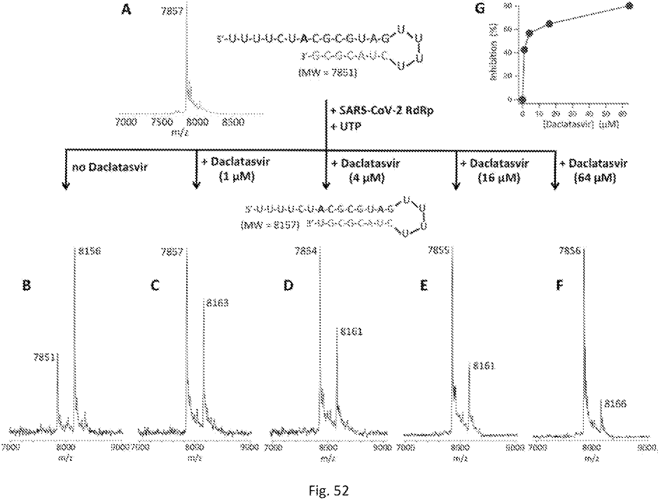

We investigated whether Daclatasvir inhibits the SARS-CoV-2 RdRp complex (nsp12/nsp7/nsp8) catalyzed reaction. Using a solution assay, we carried out a single base polymerase extension reaction in which UTP is incorporated into a RNA template-loop-primer by SARS-CoV-2 RdRp complex. We compared the efficiency of extension by UTP (FIG. 52) and Sofosbuvir triphosphate (SFV-TP, FIG. 53) in the absence and presence of various concentrations of Daclatasvir. As the concentration of Daclatasvir was increased, more extensive reduction of UTP and SFV-TP extension was obtained. These preliminary results suggest that Daclatasvir can inhibit the SARS-CoV-2 RdRp complex catalyzed reaction. The results are described below.

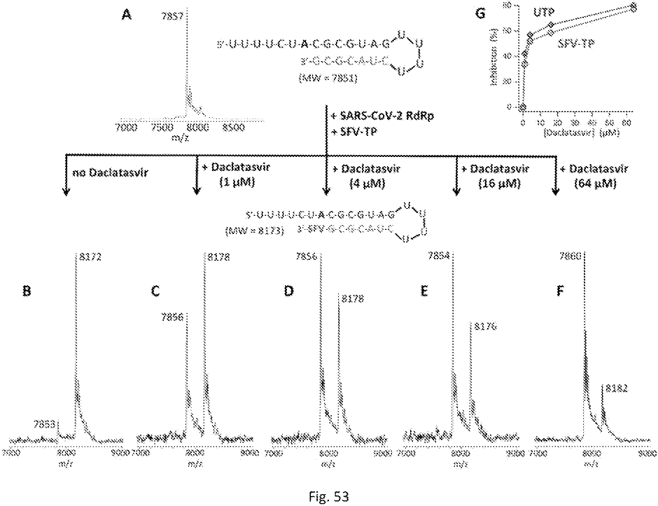

A mixture of RNA template-loop-primer (shown at the top of FIG. 52), SARS-CoV-2 pre-assembled RdRp complex (nsp12/nsp7/nsp8) and UTP was incubated in buffer solution at 30° C. for 1 hour in the absence (B) or presence of various concentrations of Daclatasvir (1, 4, 16 or 64 µM (C-F). The RNA template-loop-primer (A) and the products of the polymerase extension reaction (B-F) were analyzed by MALDI-TOF MS. Addition of daclatasvir reduced the amount of the U extended RNA product peak (8157 Da expected) in a concentration-dependent manner, with concomitant decreases in the unextended primer peak (7851 Da expected). This is visualized graphically in G. Similar results were obtained for SFV extended primer as indicated in FIG. 53 (the inset graph G compares the results for SFV from this figure and U from FIG. 52). Based on these results, it is estimated that ~2 µM Daclatasvir led to 50% inhibition of the polymerase reaction catalyzed by a 1 µM RdRp complex. These results suggest that Daclatasvir can inhibit the reaction catalyzed by the SARS-CoV-2 RdRp complex. Similar experiments were also performed for Velpatasvir. The results, which are shown in FIG. 54, indicate that increasing doses of Velpatasvir also reduce the amount of the U extended RNA product peak, though not to the same extent as Daclatasvir in this experiment. In view of the structural and functional similarity of all the HCV NS5A inhibitors, we reason that the other NS5A inhibitors should also inhibit the reaction catalyzed by the SARS-CoV-2 RdRp complex.

The detailed inhibition assays are as follows:

Assay for inhibition of SARS-CoV-2 RdRp complex catalyzed reaction by Daclatasvir. The template-loop-primer RNA (sequence shown at the top of FIG. 52) was annealed by heating to 75° C. for 3 min and cooled to room temperature in reaction buffer. The pre-assembled SARS-CoV-2 RdRp complex (nsp12/nsp7/nsp8) (1.54 µM) was incubated with appropriate concentrations of aqueous daclatasvir dihydrochloride for 15 min at room temperature in reaction buffer. The dihydrochloride salt of daclatasvir was used due to its high solubility in water. Then 5 µL of the annealed RNA template-loop-primer solution (2 µM) in reaction buffer was added to 13 µL of the RdRp-Daclatasvir mixture and incubated for an additional 10 min at room temperature. Finally, 2 µL of a solution containing 30 µM UTP or 150 µM SFV-TP was added and incubation was carried out for 1 h at 30° C. The final concentrations of the reagents in the 20 µL extension reactions were 1 µM RdRp complex (nsp12/nsp7/nsp8), 500 nM RNA template-loop-primer, 0, 1, 4, 16 or 64 µM Daclatasvir, and 3 µM UTP (FIG. 52) or 15 µM SFV-TP (FIG. 53). The 1× reaction buffer contains the following reagents: 10 mM Tris-HCl pH 8, 10 mM KCl, 2 mM MgCl₂ and 1 mM β-mercaptoethanol. Following desalting using an Oligo Clean & Concentrator kit (Zymo Research), the samples were subjected to MALDI-TOF-MS (Bruker ultrafleXtreme) analysis. The percentage of nucleotide incorporation by the RdRp complex was calculated as follows: amount of extended RNA product / (amount of remaining unextended RNA primer + amount of extended RNA product) × 100%. Percentage of inhibition of the RdRp complex-catalyzed nucleotide incorporation reaction by daclatasvir for FIG. 52 and FIG. 53 was calculated as follows: (1 - percentage of nucleotide incorporation at a given daclatasvir concentration / percentage of nucleotide incorporation in the absence daclatasvir) × 100%. A similar experiment was also performed for Velpatasvir (FIG. 54). Based on these results, the other NS5A inhibitors should also inhibit the RdRp reaction.

A concern with the use of the NS5A inhibitors described in this section, as well as other hydrophobic inhibitors to be discussed below is that they may bind to many host and viral proteins in a non-specific manner. This would reduce the likelihood of the drug acting on the target protein (e.g., RdRp or exonuclease). As one way of overcoming this complication, we have designed derivatives of these compounds with polyethylene glycol (PEG) moieties of different length attached at different positions. In FIGS. 51B and 51C, we show examples for Velpatasvir and Daclatasvir with one or two PEG modifications. In this case, the NH group on the imidazole moieties of Velpatasvir and Daclatasvir are conjugated with PEG NHS esters. In a similar way, hydroxyl or carboxyl groups of other drugs (e.g., NS3/4a and other protease inhibitors) can be modified with PEG chains of different lengths. In essence, these can behave as prodrugs.

### Example 9: NS5A Inhibitors (Daclatasvir, Velpatasvir, Pibrentasvir, Elbasvir, Ledipasvir and Ombitasvir), Protease Inhibitors (Ritonavir and Liponavir), an HIV Integrase Inhibitor (Elvitegravir) and Ebselen All Inhibit SARS-CoV-2 Exonuclease (nsp14/nsp10) Activity

In Examples 6 and 7, we identified nucleotides that, once incorporated into RNA, showed some resistance to excision by the SARS-CoV-2 proofreading exonuclease (nsp14/nsp10). This would increase their likelihood of serving as permanent polymerase terminators that interfere with viral replication. However, other non-nucleoside, non-nucleotide drugs may exert an inhibitory effect on the exonuclease itself through a distinct mechanism. In this example, we tested a variety of drugs for their ability to selectively inhibit the SARS-CoV-2 proofreading exonuclease.

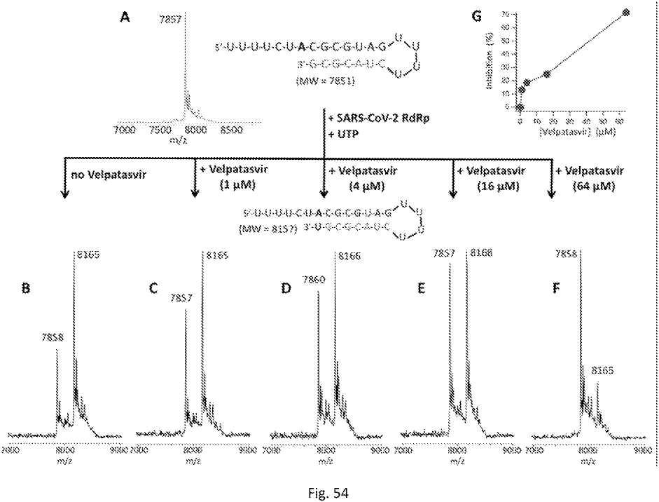

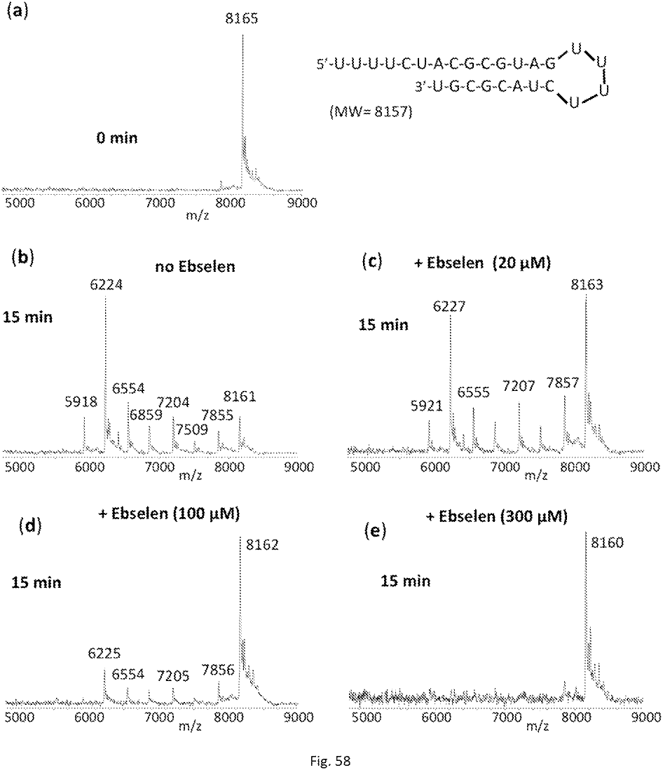

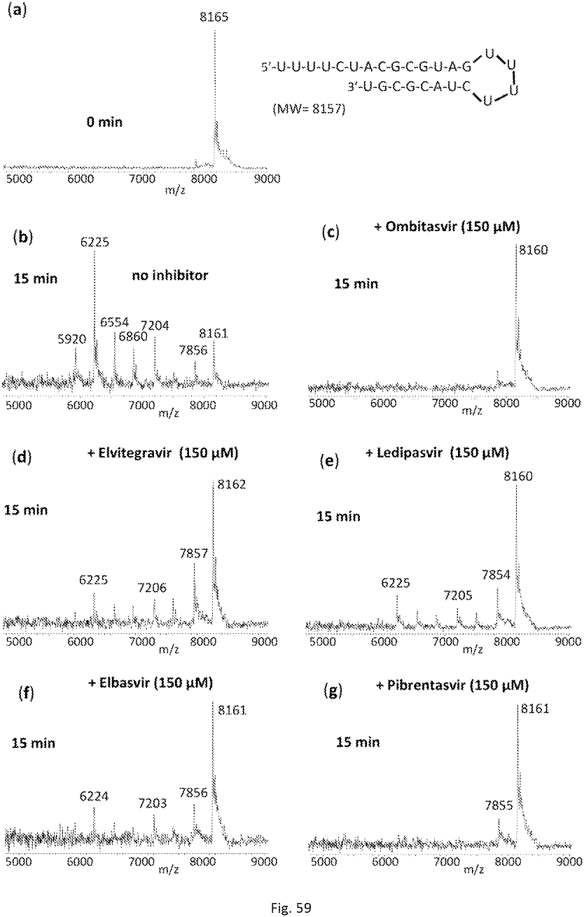

We first tested the NS5A inhibitors Velpatasvir and Daclatasvir, which we already showed in Example 7 can impede the RdRp reaction, for their ability to inhibit the SARS-CoV-2 exonuclease (FIG. 55). A mixture of 500 nM of RNA template-loop-primer (shown at the top of the figure) and 30 nM SARS-CoV-2 pre-assembled exonuclease complex (nsp14/nsp10) was incubated in buffer solution at 37° C. for 15 min in the absence (b) and presence of Daclatasvir at 5 µM (c) and 150 µM (d) or Velpatasvir at 50 µM (e) and 150 µM (f). The RNA template-loop-primer (a) and the products of the exonuclease reaction (b-f) were analyzed by MALDI-TOF MS. The signal intensity was normalized to the highest peak. The accuracy for m/z determination is ± 10 Da. Reaction conditions were selected to yield efficient RNA fragmentation due to exonuclease activity as seen by MALDI-TOF-MS analysis in (b). The peak at 8161 Da corresponds to the RNA template-loop-primer (8157 Da expected) and peaks at lower molecular weights, such as 7855, 7511, 7204, 6860, 6554, 6225 and 5919 Da correspond to fragments after cleavage of 1, 2, 3, 4, 5, 6 and 7 nucleotides from the 3′-end respectively (b). Addition of 5 µM of Daclatasvir (c) or 50 µM of Velpatasvir (e) shows inhibition of the exonuclease activity as seen by MALDI-TOF-MS by the presence of the larger template-loop-primer peak (8162 Da or 8164 Da) compared to absence of Daclatasvir or Velpatasvir (b). With further increase of Daclatasvir or Velpatasvir to a concentration of 150 µM (d, f), the template-loop-primer peak (8159 Da or 8165 Da) increases and fragmentation peaks are reduced indicating increased inhibition of exonuclease activity. Four additional NS5A inhibitors (150 µM Ombitasvir, Ledipasvir, Elbasvir and Pibrentasvir) were also able to inhibit the exonuclease (FIGS. 59c, e, f and g respectively), as was the HIV integrase inhibitor Elvitegravir (FIG. 59d). Thus, Velpatasvir and Daclatasvir can inhibit both the SARS-CoV-2 RdRp (using the nsp12/nsp7/nsp8 complex in solution assays) and the SARS-CoV-2 exonuclease (using the nsp14/nsp10 complex in solution assays). Two of the above inhibitors, Ombitasvir and Pibrentasvir, were also tested at lower concentrations (1 µM and 10 µM for Ombitasvir and 0.1 µM and 10 µM for Pibrentasvir) (FIG. 60). Addition of 1 µM of Ombitasvir (c) or 0.1 µM of Pibrentasvir (d) shows inhibition of the exonuclease activity as seen by MALDI-TOF-MS by the presence of the larger template-loop-primer peak (8162 Da or 8164 Da) compared to absence of Ombitasvir or Pibrentasvir (b). With further increase of Ombitasvir or Pibrentasvir to a concentration of 10 µM (e, f), the template-loop-primer peak (8160 Da) increases and fragmentation peaks are reduced indicating significantly increased inhibition of exonuclease activity. In view of the structural and functional similarity of all the NS5A inhibitors, we reason that the other NS5A inhibitors should also inhibit the SARS-CoV-2 exonuclease reaction.

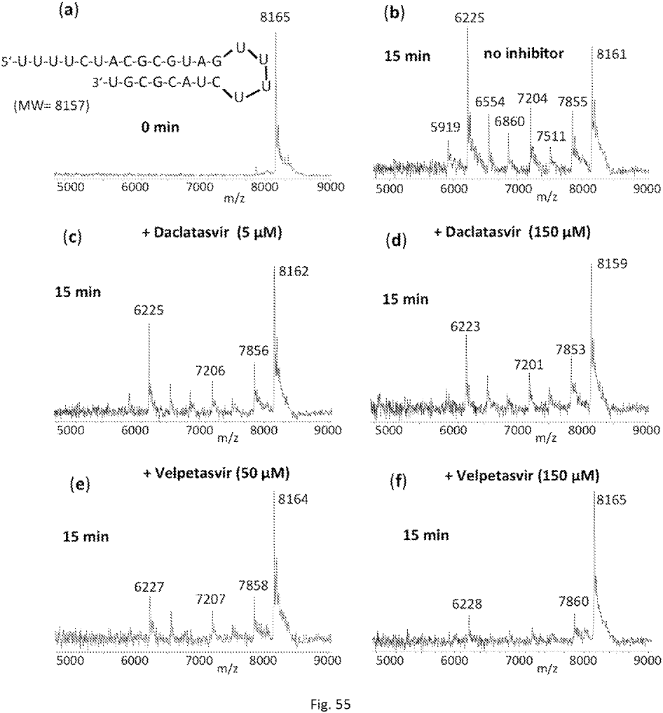

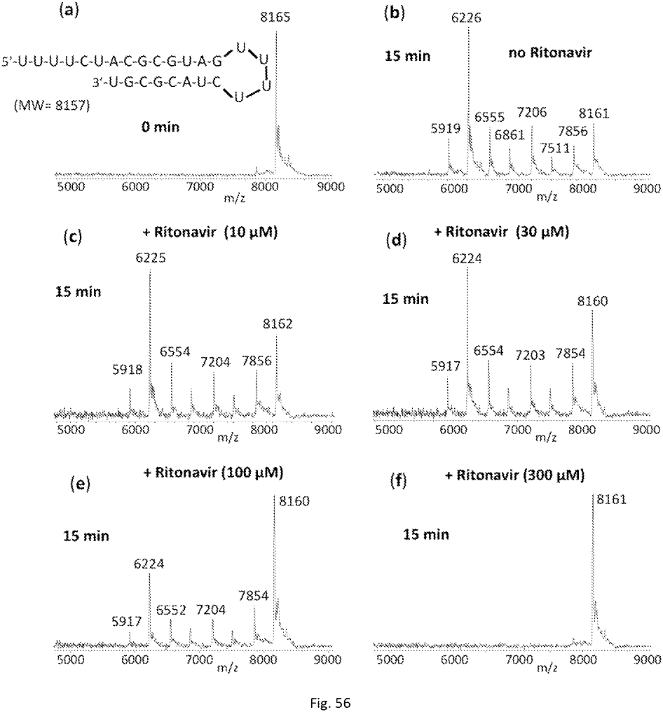

We next tested the known protease inhibitors Ritonavir and Lopinavir for their ability to inhibit the SARS-CoV-2 exonuclease. The result for Ritonavir is shown in FIG. 56. A mixture of 500 nM of RNA template-loop-primer (shown at the top of the figure) and 30 nM SARS-CoV-2 pre-assembled exonuclease complex (nsp14/nsp10) was incubated in buffer solution at 37° C. for 15 min in the absence (b) and presence of Ritonavir at 10 µM (c), 30 µM (d), 100 µM (e) and 300 µM (f). The RNA template-loop-primer (a) and the products of the exonuclease reaction (b-f) were analyzed by MALDI-TOF MS. The signal intensity was normalized to the highest peak. The accuracy for m/z determination is ± 10 Da. Reaction conditions were selected to yield efficient RNA fragmentation due to exonuclease activity as seen by MALDI-TOF-MS analysis in (b). The peak at 8161 Da corresponds to the RNA template-loop-primer (8157 Da expected) and peaks at lower molecular weights, such as 7856, 7511, 7206, 6861, 6555, 6226 and 5919 Da correspond to fragments after cleavage of 1, 2, 3, 4, 5, 6 and 7 nucleotides from the 3′-end respectively (b). Addition of 10 µM (c) of Ritonavir shows inhibition of the exonuclease activity as seen by MALDI-TOF-MS by the presence of the larger template-loop-primer peak (8162 Da) compared to absence of Ritonavir (b). With further increase of Ritonavir to concentrations of 30 µM (d) and 100 µM (e), the template-loop-primer peak (8160 Da) increases and fragmentation peaks are further reduced. At 300 µM of Ritonavir (f), complete inhibition of exonuclease activity was observed as evident by the similarity of the MS in the absence of exonuclease (a). The result for Lopinavir is shown in FIG. 57. A mixture of 500 nM of RNA template-loop-primer (shown at the top of the figure) and 30 nM SARS-CoV-2 pre-assembled exonuclease complex (nsp14/nsp10) was incubated in buffer solution at 37° C. for 15 min in the absence (b) and presence of Lopinavir at 10 µM (c), 30 µM (d), 100 µM (e) and 300 µM (f). The RNA template-loop-primer (a) and the products of the exonuclease reaction (b-f) and analyzed in the same way. The peak at 8161 Da corresponds to the RNA template-loop-primer (8157 Da expected) and peaks at lower molecular weights, such as 7856, 7511, 7206, 6861, 6555, 6226 and 5919 Da correspond to fragments after cleavage of 1, 2, 3, 4, 5, 6 and 7 nucleotides from the 3′-end respectively (b). Addition of 10 µM (c) of Lopinavir shows a small inhibition of the exonuclease activity as seen by MALDI-TOF-MS by the presence of the slightly larger template-loop-primer peak (8160 Da) compared to absence of Lopinavir (b). With further increase of Lopinavir to concentrations of 30 µM (d) and 100 µM (e), the template-loop-primer peak (8160 Da) increases and fragmentation peaks are further reduced. At 300 µM of Lopinavir (f), complete inhibition of exonuclease activity was observed as evident by the similarity of the MS in the absence of exonuclease (a).

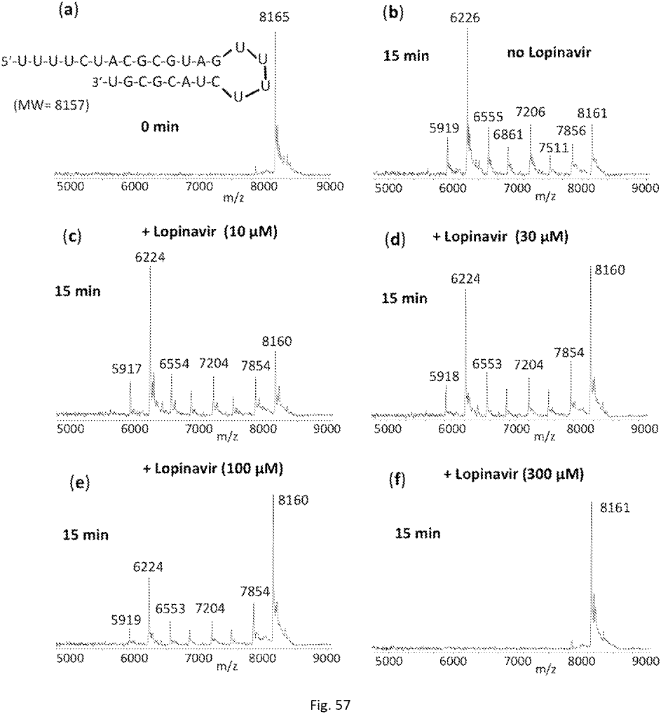

We next tested the SARS-CoV-2 major protease (M^{pro}) inhibitor Ebselen, which has been reported to inhibit the exonuclease (Baddock et al 2020) in our assay (FIG. 58). A mixture of 500 nM of RNA template-loop-primer (shown at the top of the figure) and 30 nM SARS-CoV-2 pre-assembled exonuclease complex (nsp14/nsp10) was incubated in buffer solution at 37° C. for 15 min in the absence (b) and presence of Ebselen at 20 µM (c), 100 µM (d) and 300 µM (e). The RNA template-loop-primer (a) and the products of the exonuclease reaction (b-e) were analyzed by MALDI-TOF MS. The signal intensity was normalized to the highest peak. The accuracy for m/z determination is ±10 Da. Reaction conditions were selected to yield efficient RNA fragmentation due to exonuclease activity as seen by MALDI-TOF-MS analysis in (b). The peak at 8161 Da corresponds to the RNA template-loop-primer (8157 Da expected) and peaks at lower molecular weights, such as 7855, 7509, 7204, 6859, 6554, 6224 and 5918 Da correspond to fragments after cleavage of 1, 2, 3, 4, 5, 6 and 7 nucleotides from the 3′-end respectively (b). Addition of 20 µM (c) of Ebselen shows inhibition of the exonuclease activity as seen by MALDI-TOF-MS by the presence of the larger template-loop-primer peak (8163 Da) compared to absence of Ebselen (b). With increase of Ebselen to a concentration of 100 µM, the template-loop-primer peak (8162 Da) dominates the MS and only small fragmentation peaks were observable (d). At 300 µM of Ebselen (e), almost complete inhibition of exonuclease activity was observed as evident by the similarity of the MS in the absence of exonuclease (a).

### REFERENCES

Agostini et al (2019) J Virol 93:e01348-19.
Akyurek et al (2001) Molec Ther 3:779-786.
Alanazi et al (2019) ACS Med Chem Lett 10:2-5.
Anilkumar et al (2015) Nucleos Nucleot Nucl Acids 34:92-102.
Arnold et al (2012) PLoS Pathog 8:e1003030.
Arup et al (1992) Biochem 31:9636-9641.
Ascher et al (2014) Sci Rep 4:4765.
Baddock et al (2020) bioRxiv 10.1101/2020.08.13.248211.
Birkus et al (2016) Antimicrob Agents Chemother 60:316-322.
Blasiak et al (2020) AIChE Bioeng Transl Med.
https://aiche.onlinelibrary.wiley.com/doi/10.1002/btm2.10196. Bouvet et al (2012) Proc Natl Acad Sci USA 109:9372-9377. Buonaguro et al (2020) J Transl Med 18:185.
Camerman et al (1990) Proc Natl Acad Sci USA 87:3534-3537.
Canard & Sarfati (1994) Gene 148:1-6.
Chan et al (2020) J Infect. In press.
https://doi.org/10.1016/j.jinf.2020.12.021
Chien et al (2020a) bioRxiv. https://doi.org/10.1101/2020.03.18.997585.
Chien et al (2020b) J Proteome Res 19:4690-4697.
Cho et al (2012) Bioorg Med Chem Lett 22:2705-2707.
Cundy (1999) Clin Pharmacokinet 36:127-143.
De Clercq (2002) Antivir Res 55:1-13.
De Clercq (2016) Biochem Pharmacol 119:1-7.
De Clercq & Field (2006) Br J Pharmacol 147:1-11.
De Clercq & Li (2016) Clin Microbiol Rev 29:695-747.
Deval et al (2014) Curr Opin Virol 9:1-7.
Dustin et al (2016) Clin Microbiol Infect 22:826-832.
Dutartre et al (2006) Antimicrob Agents Chemother 50:4161-4169.
Eastman et al (2020) ACS Cent Sci 6:672-683.
Eckerle et al (2010) PLoS Pathogens 6:e1000896.
Elfiky (2020a) Life Sci 248:117477.
Elfiky (2020b) Life Sci 253:117592.
Elfiky et al (2017) J Med Virol 89:1040-1047.
Eltahla et al (2015) Viruses 7:5206-5224.
Eslami et al (2020) J Antimicrob Chemother.
https://doi.org/10.1093/jac/dkaa331.
Faletto et al (1997) Antimicrob Agents Chemother 41:1099-1107.
Fearns & Deval (2016) Antiviral Res 134:63-76.
Ferron (2018) Proc Natl Acad Sci USA 115:E162-E171.
Fintelman-Rodrigues et al (2020) Antimicrob Agents Chemother 64:e00825-20.
Fung et al (2014) Antimicrob Agents Chemother 58:3636-3645.
Furman et al (1986) Proc Natl Acad Sci USA 83:8333-8337.
Gao et al (2020) Science 368:779-782.
Gitto et al (2017) J Viral Hepat 24:180-186.
Gordon et al (2020a) J Biol Chem 295:4773-4779.
Gordon et al (2020b) J Biol Chem 295:6785-6797.
Guo et al (2008) Proc Natl Acad Sci USA 105:9145-9150.
Hillen et al (2020) Nature 584:154-156.
Ho & Hitchcock (1989) Antimicrob Agents Chemother 33:844-849.
Holshue et al (2020) N Engl J Med 382:929-936.
Huang et al (1992) J Biol Chem 267:2817-2822.
Hung et al (2019) Commun Biol 2:469.
Hung et al (2020) Lancet 395:1695-1704.
Jácome et al (2020) Sci Rep 10:9294.
Jockusch et al (2020a) bioRxiv.
https://doi.org/10.1101/2020.04.23.058776.
Jockusch et al (2020b) Antiviral Res 180:104857.
Jockusch et al (2020c) Sci Rep 10:16577.
Jockusch et al (2020d) bioRxiv. https://doi.org/10.1101/2020.04.03.022939.
Joshi et al (2021) Int J Infect Dis 102:501-508.
Ju et al (2020a) bioRXiv. https://doi.org/10.1101/2020.01.30.927574.
Ju et al (2020b) Pharmacol Res Perspect 8:e00674.
Ju et al (2006) Proc Natl Acad Sci USA 103:19635-19640.
Ju et al (2003) U.S. Pat. 6,664,079 (2003).
Kasgari et al (2020) J Antimicrob Chemother.
https://doi.org/10.1093/jac/dkaa332.
Kayali & Schmidt (2014) Pharmgenom Pers Med 7:387-398.
Kirchdoerfer & Ward (2019) Nature Commun 10:2342.
Kumar et al (2005) Nucleos Nucleot Nucl 24:401-408.
Lanier et al (2010) Viruses 2:2740-2762.
Lauridsen et al (2012) ChemBioChem 13:19-25.
Lim et al (2006) Arch Int Med 166:49-56.
Lou (2013) J Clin Translat Hepatol 1:33-38.
Ma et al (2015) Proc Natl Acad Sci USA 112:9436-9441.
Magee et al (2005) Antimicrob Agents Chemother 49:3153-3162.
Margolis et al (2014) J Med Toxicol 10:26-39.
Matthews (2006) Cell Mol Life Sci 71:4403-4420.
Matthews & Boehme (1988) Rev Infect Dis 10:S490-S494.
Mazzucco et al (2008) Antimicrob Agents Chemother 52:598-605.
McKenna et al (1989) ACS Symposium Series 401:1-16.
Mesci et al (2020) bioRxiv. https://doi.org/10.1101/2020.05.30.125856.
Minskaia et al (2006) Proc Natl Acad Sci USA 103:5108-5113.
Mitsuya et al (1985) Proc Natl Acad Sci USA 82:7096-7100.
Mitsuya et al (1990) Science 249:1533-1544.
Murakami et al (2010) J Biol Chem 285:34337-34347.
Narayanan & Nair (2021) Int J Biol Macromol 168:272-278.
Nguyenla et al (2020) bioRxiv. https://doi.org/10.1101/2020.09.18.302398.
Oberg (2006) Antiviral Res 71:90-95.
Olagunju et al (2021) Trials 22:3
Quercia et al (2018) J Aquir Immune Defic Syndr 78:125-135.
Quezada & Kane (2009) Open Biochem J 3:39-48.
Ray et al (2002) J Biol Chem 277:40479-40490.
Rivkina & Rybalov (2002) Pharmacother 22:721-737.
Roberts et al (2008) Hepatol 48:398-406.
Ross et al (2011) J Org Chem 76:8311-8319.
Sacramento et al (2020) bioRxiv.
https://doi.org/10.1101/2020.06.15.153411.
Sachin et al (2016) J Org Chem 81:8331-8342.
Sayad et al (2020) Arch Med Res S0188-4409:30551-8.
https://doi.org/10.1016/j.arcmed.2020.04.018.
Sadeghi et al (2020) J Antimicrob Chemother
https://doi.org/10.1093/jac/dkaa334.
Selisko et al (2018) Viruses 10:59.
Shannon et al (2020) Antiviral Res 178:104793.
Sheahan et al (2020) Sci Transl Med.
https://doi.org/10.1126/scitranslmed.abb5883.
Siegel et al (2017) J Med Chem 60:1648-1661.
Smith et al (2013) PLoS Pathogens 9:e1003565.
Smith et al (2016) Ann Pharmacother 50:39-46.
Sofia et al (2010) J Med Chem 53:7202-7218.
Sood et al (2005) J Am Chem Soc 127:2394-2395.
Subissi et al (2014) Proc Natl Acad Sci USA 111:E3900-E3909.
Tabor & Richardson (1995) Proc Natl Acad Sci USA 92:6339-6343.
Tchesnokov et al (2019) Viruses 11:326.
Tchesnokov et al (2008) J Biol Chem 283:34218-34228.
te Velthuis (2014) Cell Mol Life Sci 71:4403-4420.
Trost et al (2015) Antivir Res 117:115-121.
Wang et al (2020) Cell Res 30:269-271.
Warren et al (2014) Nature 508:402-405.
World Hepatitis Alliance press release.
https://www.worldhepatitisalliance.org/latest
news/infohep/3548907/hepatitis-c-drugs-may-offer-inexpensivetreatment-option-covid-19.
Xu et al (2017) Anti-Viral Therapy 22:587-597.
Yarchoan et al (1986) Lancet 327:575-580.
Yin et al (2020) Science 368:1499-1504.
Yuan et al (2020) Nature Microbiol 5:1439-1448.
Zhu et al (2020) N Eng J Med 382:727-733.
Zumla et al (2016) Nat Rev Drug Discovery 15:327-347.
https://www.genetex.com/MarketingMaterial/Index/SARS-CoV-2_Genome_and_Proteome.
https://www.eurekalert.org/pub_releases/2020-08/oupu-sdm082220.php.

What is claimed is:

1. A composition comprising at least one of the following compounds for the treatment of viral infection caused by one or more viruses selected from the group consisting of SARS-CoV-2, SARS-CoV, MERS-CoV, Marburg virus, Ebola virus and influenza virus: 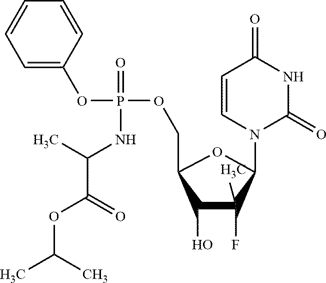 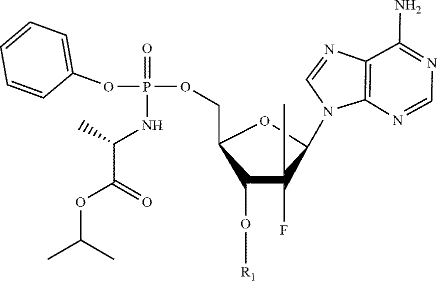 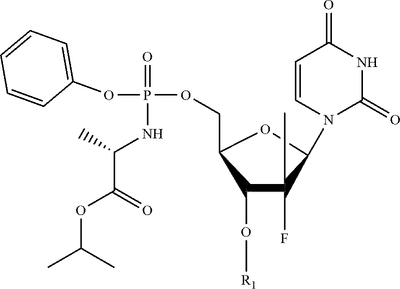 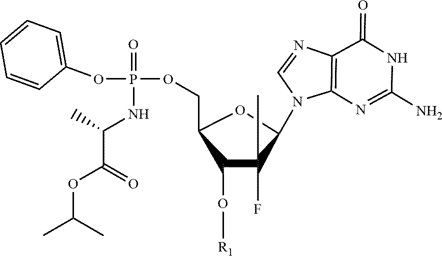 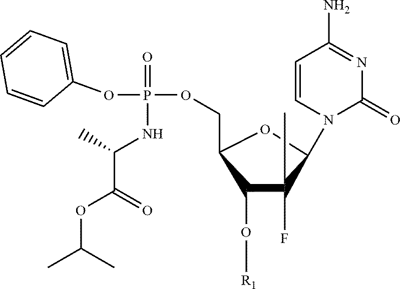 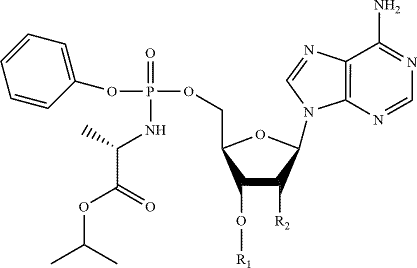 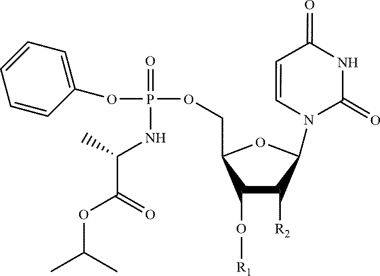 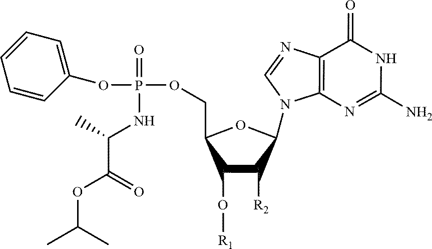 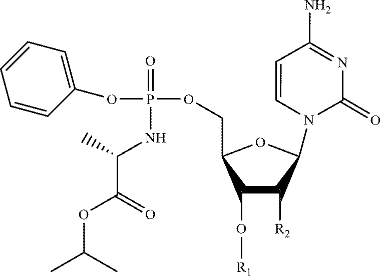
wherein R₁ is H, methyl (CH₃), ethyl (CH₂CH₃), propyl (CH₂CH₂CH₃), allyl (CH₂CH=CH₂), propargyl (CH₂C=CH), methoxymethyl (CH₂OCH₃), methylthiomethyl (CH₂SCH₃), azidomethyl (CH₂-N₃), or a small chemical group that does not prevent the recognition of the nucleotide analogue as a substrate by the viral polymerase,
wherein R₂ is H, OH, F, or OCH₃, and wherein the nucleobase in each said compound is a natural nucleobase or a base analog thereof selected from the group consisting of 7-deaza-G, 7-deaza-A, inosine, and derivatives thereof.

2. A composition comprising at least one of the following compounds for the treatment of viral infection caused by one or more viruses selected from the group consisting of SARS-CoV-2, SARS-CoV, MERS-CoV, hepatitis C virus, Marburg virus, Ebola virus and influenza virus comprising: 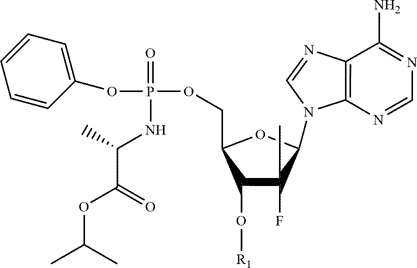 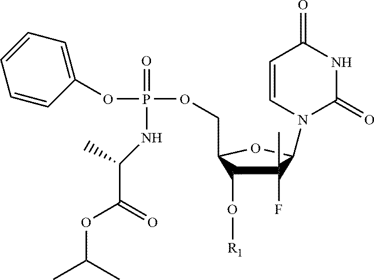 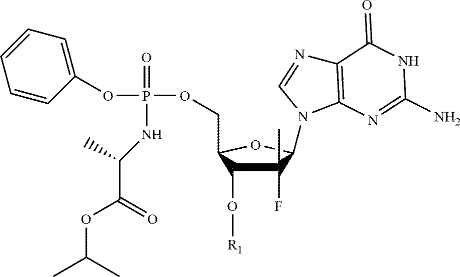 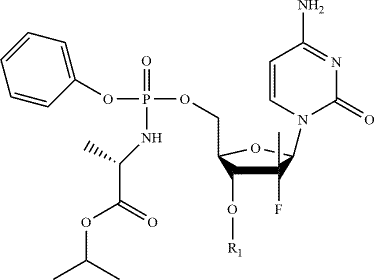 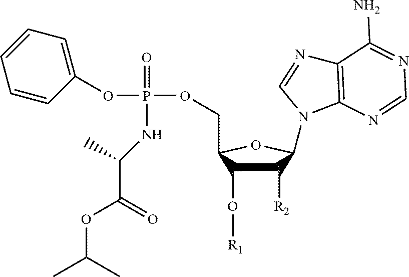 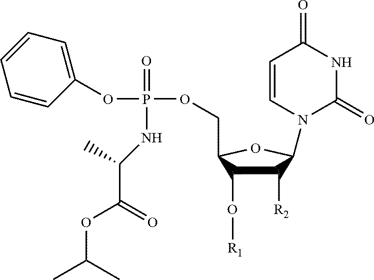 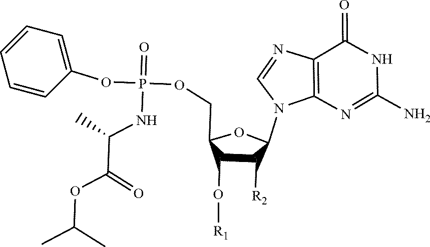 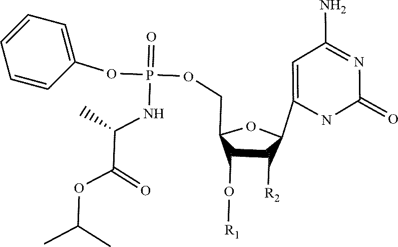
wherein R₁ is H, methyl (CH₃), ethyl (CH₂CH₃), propyl (CH₂CH₂CH₃), allyl (CH₂CH=CH₂), propargyl (CH₂C=CH), methoxymethyl (CH₂OCH₃), methylthiomethyl (CH₂SCH₃), azidomethyl (CH₂-N₃), or a small chemical group that does not prevent the recognition of the nucleotide analogue as a substrate by the viral polymerase, wherein R₂ is H, OH, F, or OCH₃, and wherein the nucleobase in each said compound is a natural nucleobase or a base analog thereof selected from the group consisting of 7-deaza-G, 7-deaza-A, and inosine, and derivatives thereof.

3. A composition comprising at least one of the following compounds for the treatment of viral infection caused by one or more viruses selected from the group consisting of SARS-CoV-2, SARS-CoV, MERS-CoV, Marburg virus, Ebola virus and influenza virus: EPCLUSA (Sofosbuvir/Velpatasvir), Sofosbuvir/Daclatasvir, 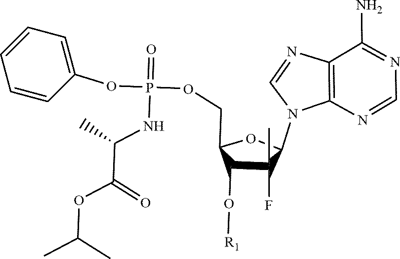 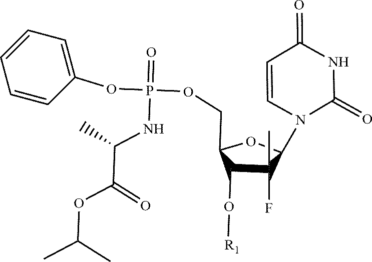 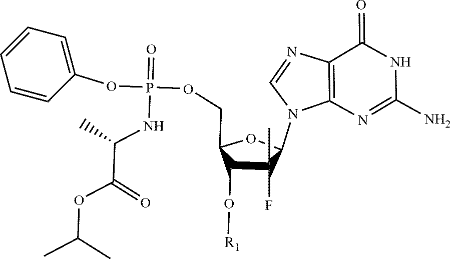 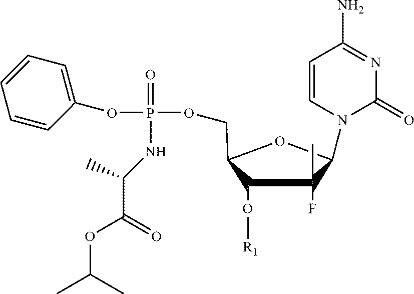 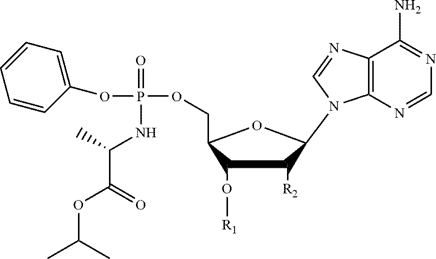 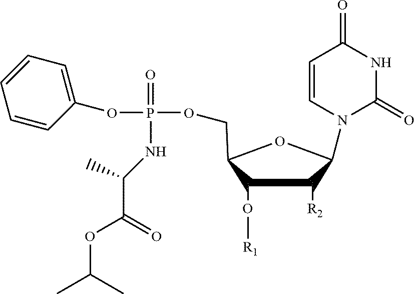 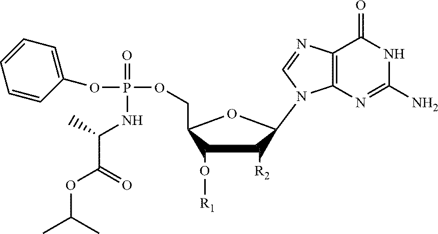 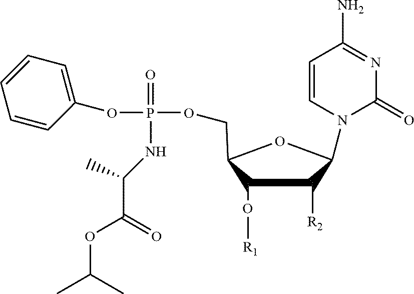
wherein R₁ is H, methyl (CH₃), ethyl (CH₂CH₃), propyl (CH₂CH₂CH₃), allyl (CH₂CH=CH₂), propargyl (CH₂C=CH), methoxymethyl (CH₂OCH₃), methylthiomethyl (CH₂SCH₃), azidomethyl (CH₂-N₃), or a small chemical group that does not prevent the recognition of the nucleotide analogue as a substrate by the viral polymerase,
wherein R₂ is H, OH, F, or OCH₃, and wherein the nucleobase in each said compound is a natural nucleobase or a base analog thereof selected from the group consisting of 7-deaza-G, 7-deaza-A, and inosine, and derivatives thereof.

4. A composition comprising at least one of the following compounds for the treatment of viral infection caused by one or more viruses selected from the group consisting of SARS-CoV-2, SARS-CoV, MERS-CoV, hepatitis C virus, Marburg virus, Ebola virus and influenza virus: 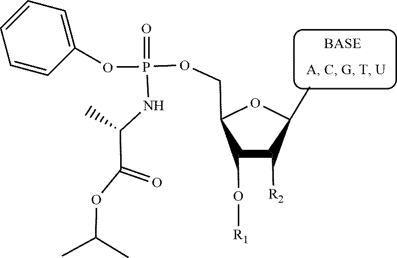 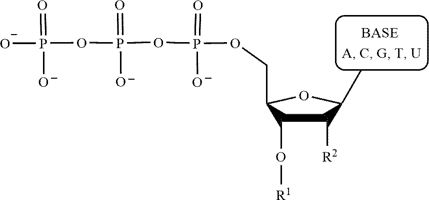
wherein R₁ is H, methyl, or a small chemical group that does not prevent the recognition of the nucleotide analogue as a substrate by the viral polymerase,
wherein R₂ is OH, F, H, or -O-ester,
wherein BASE is A, C, G, T, U or derivatives thereof, and
wherein the compounds depicted on the left are prodrugs of the active forms of the compounds depicted on the right.

5. A composition comprising at least one of the following compounds for the treatment of viral infection caused by one or more viruses selected from the group consisting of SARS-CoV-2, SARS-CoV, MERS-CoV, hepatitis C virus, Marburg virus, Ebola virus and influenza virus: 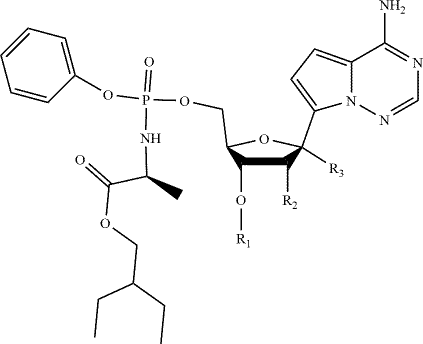 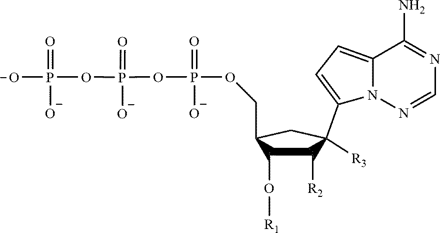
wherein R₁ is H, methyl, or a small ester that does not prevent the recognition of the nucleotide analogue as a substrate by the viral polymerase,
wherein R₂ is OH, F, H, or -O-ester,
wherein R₃ is F, methyl, or ethyl, and
wherein the compounds depicted on the left are prodrugs of the active forms of the compounds depicted on the right.

6. A composition comprising at least one of the following compounds for the treatment of viral infection caused by one or more viruses selected from the group consisting of SARS-CoV-2, SARS-CoV, MERS-CoV, hepatitis C virus, Marburg virus, Ebola virus and influenza virus: 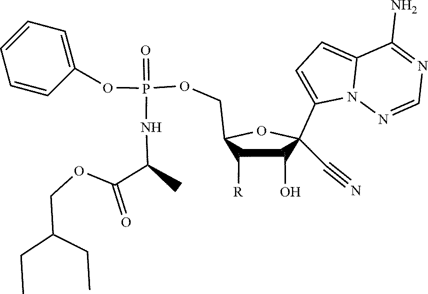
wherein R is H, F, or NH₂.

7. A composition comprising at least one of the following compounds for the treatment of viral infection caused by one or more viruses selected from the group consisting of SARS-CoV-2, SARS-CoV, MERS-CoV, hepatitis C virus, Marburg virus, Ebola virus and influenza virus: 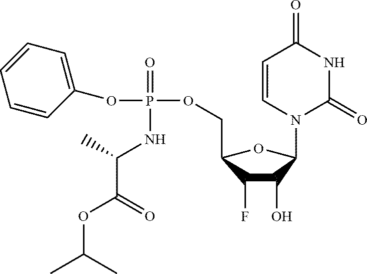 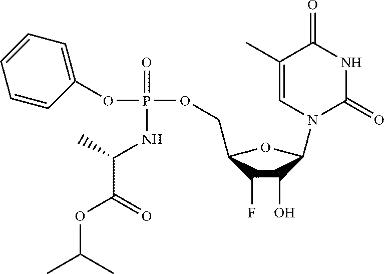
.

8. A composition comprising at least one of the following compounds for the treatment of viral infection caused by one or more viruses selected from the group consisting of SARS-CoV-2, SARS-CoV, MERS-CoV, hepatitis C virus, Marburg virus, Ebola virus and influenza virus: 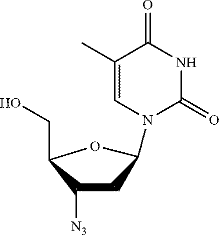 [SMILES: Cc1cn([C@H]2C[C@@H](N=[N+]=[N-])[C@@H](CO)O2)c(=O)[nH]c1=O] 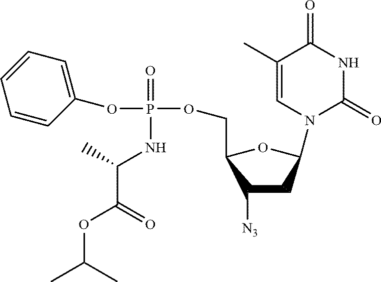 [SMILES: Cc1cn([C@H]2C[C@@H](N=[N+]=[N-])[C@@H](COP(=O)(N[C@@H](C)C(=O)OC(C)C)Oc3ccccc3)O2)c(=O)[nH]c1=O] 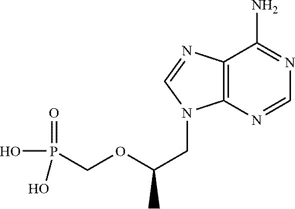 [SMILES: C[C@H](Cn1cnc2c(N)ncnc21)OCP(=O)(O)O] 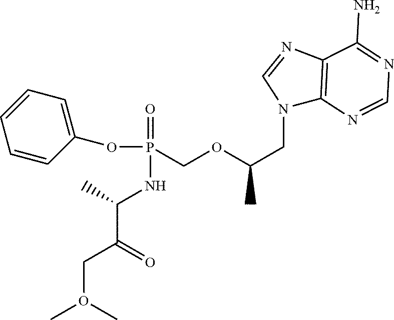
.

9. A composition comprising at least one of the following compounds for the treatment of viral infection caused by one or more viruses selected from the group consisting of SARS-CoV-2, SARS-CoV, MERS-CoV, hepatitis C virus, Marburg virus, Ebola virus and influenza virus: 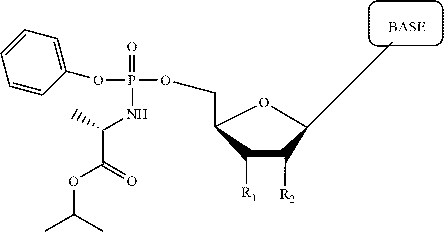
wherein BASE is A, C, G, T, U or derivatives thereof,
wherein R₁ is H, methyl, F, N₃, or a small chemical group that does not prevent the recognition of the nucleotide analogue as a substrate by the viral polymerase,
wherein R₂ = H, OH, F, N₃, or -O-ester, and
wherein R₃ = F, methyl, or ethyl.

10. A composition comprising at least one of the following compounds for the treatment of viral infection caused by one or more viruses selected from the group consisting of SARS-CoV-2, SARS-CoV, MERS-CoV, Marburg virus, Ebola virus and influenza virus: 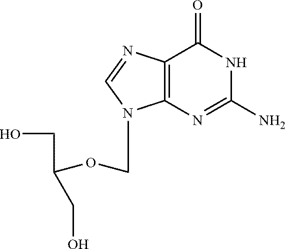 [SMILES: Nc1nc2c(ncn2COC(CO)CO)c(=O)[nH]1] 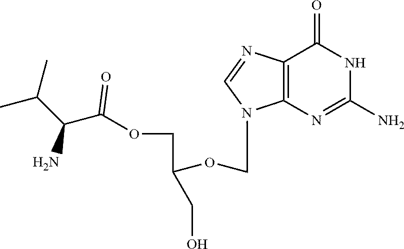 [SMILES: CC(C)[C@H](N)C(=O)OCC(CO)OCn1cnc2c(=O)[nH]c(N)nc21] 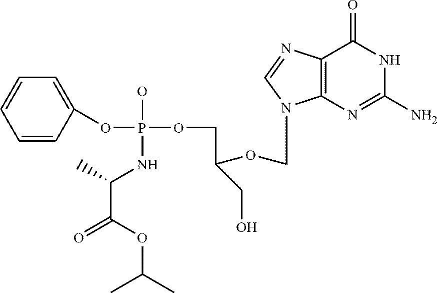
.

11. A composition comprising at least one of the following compounds for the treatment of viral infection caused by one or more viruses selected from the group consisting of SARS-CoV-2, SARS-CoV, MERS-CoV, Marburg virus, Ebola virus and influenza virus: 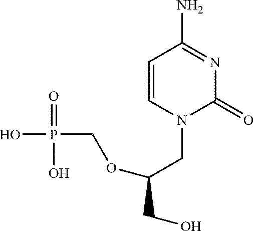 [SMILES: Nc1ccn(C[C@@H](CO)OCP(=O)(O)O)c(=O)n1] 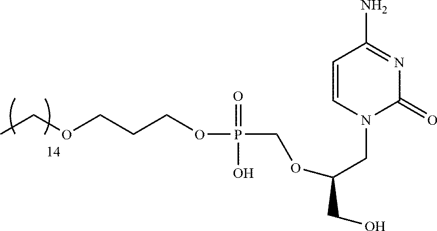 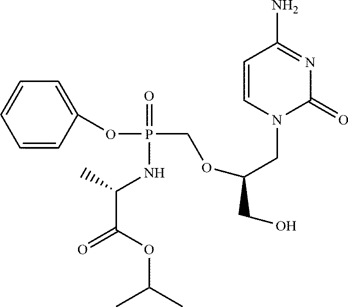 [SMILES: CC(C)OC(=O)[C@H](C)NP(=O)(CO[C@H](CO)Cn1ccc(N)nc1=O)Oc1ccccc1]
.

12. A composition comprising at least one of the following compounds for the treatment of viral infection caused by one or more viruses selected from the group consisting of SARS-CoV-2, SARS-CoV, MERS-CoV, Marburg virus, Ebola virus and influenza virus: 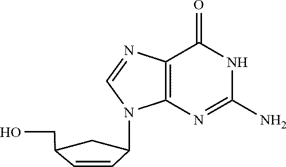 [SMILES: Nc1nc2c(ncn2C2C=CC(CO)C2)c(=O)[nH]1] 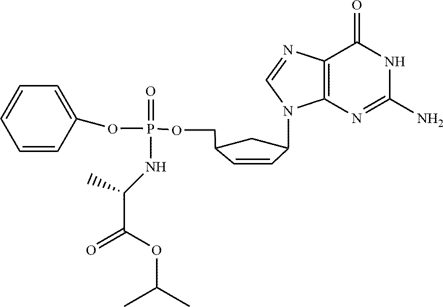 [SMILES: CC(C)OC(=O)[C@H](C)NP(=O)(OCC1C=CC(n2cnc3c(=O)[nH]c(N)nc32)C1)Oc1ccccc1] 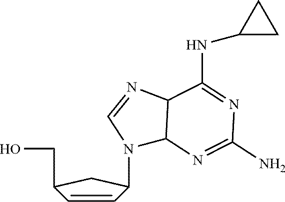 [SMILES: NC1=NC2C(N=CN2C2C=CC(CO)C2)C(NC2CC2)=N1] 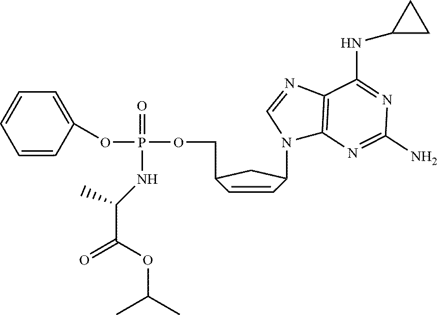 [SMILES: CC(C)OC(=O)[C@H](C)NP(=O)(OCC1C=CC(n2cnc3c(NC4CC4)nc(N)nc32)C1)Oc1ccccc1]
.

13. A composition comprising at least one of the following compounds for the treatment of viral infection caused by one or more viruses selected from the group consisting of SARS-CoV-2, SARS-CoV, MERS-CoV, Marburg virus, Ebola virus and influenza virus: 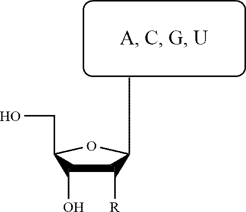 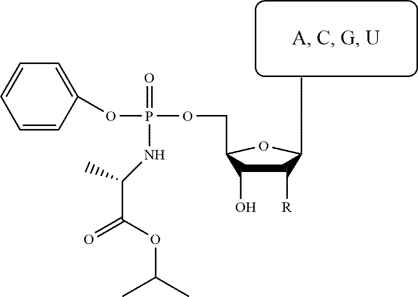
wherein R is F, OMe, NH₂, or OCH₂OCH₃.

14. A composition comprising at least one of the following compounds for the treatment of viral infection caused by one or more viruses selected from the group consisting of SARS-CoV-2, SARS-CoV, MERS-CoV, Marburg virus, Ebola virus and influenza virus: 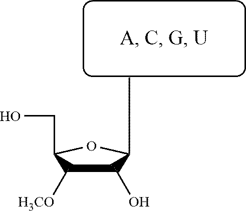 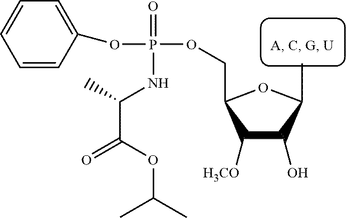
.

15. A composition comprising at least one of the following compounds for the treatment of viral infection caused by one or more viruses selected from the group consisting of SARS-CoV-2, SARS-CoV, MERS-CoV, Marburg virus, Ebola virus and influenza virus: 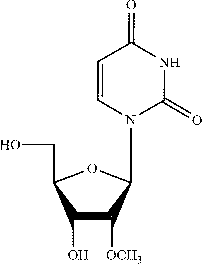 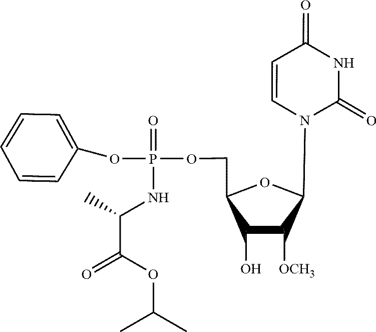
.

16. A composition comprising at least one of the following compounds for the treatment of viral infection caused by one or more viruses selected from the group consisting of SARS-CoV-2, SARS-CoV, MERS-CoV, Marburg virus, Ebola virus and influenza virus: 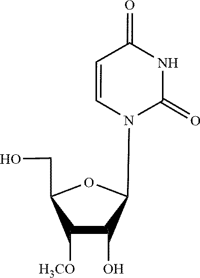 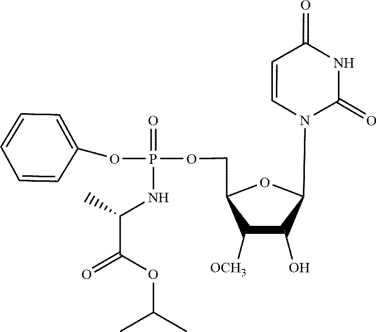
.

17. A composition comprising at least one of the following compounds for the treatment of viral infection caused by one or more viruses selected from the group consisting of SARS-CoV-2, SARS-CoV, MERS-CoV, Marburg virus, Ebola virus and influenza virus: 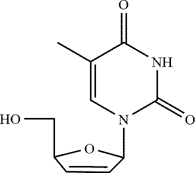 [SMILES: Cc1cn(C2C=CC(CO)O2)c(=O)[nH]c1=O] 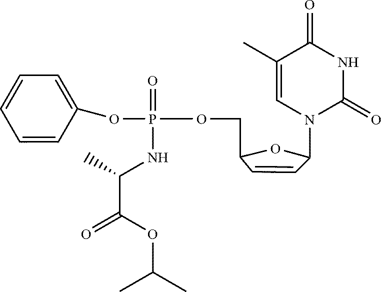 [SMILES: Cc1cn(C2C=CC(COP(=O)(N[C@@H](C)C(=O)OC(C)C)Oc3ccccc3)O2)c(=O)[nH]c1=O]
.

18. A composition comprising at least one of the following compounds for the treatment of viral infection caused by one or more viruses selected from the group consisting of SARS-CoV-2, SARS-CoV, MERS-CoV, Marburg virus, Ebola virus and influenza virus: 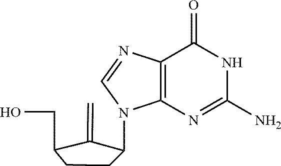 [SMILES: C=C1C(CO)CCC1n1cnc2c(=O)[nH]c(N)nc21] 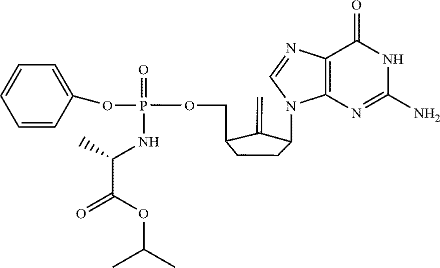 [SMILES: C=C1C(COP(=O)(N[C@@H](C)C(=O)OC(C)C)Oc2ccccc2)CCC1n1cnc2c(=O)[nH]c(N)nc21]
.

19. A composition comprising at least two of the compounds from claims 1 - 18 for the treatment of viral infection caused by one or more viruses selected from the group consisting of SARS-CoV-2, SARS-CoV, MERS-CoV, Marburg virus, Ebola virus and influenza virus.

20. A composition comprised of at least three of the compounds from claims 1 - 18 for the treatment of viral infection caused by one or more viruses selected from the group consisting of SARS-CoV-2, SARS-CoV, MERS-CoV, the Marburg virus, Ebola virus and influenza virus.

21. A method for treating a viral infection caused by a coronavirus in a human subject afflicted with the viral infection comprising administering to the human subject a therapeutically active dose of an RdRp inhibitor selected from the group consisting of Sofosbuvir, Remdesivir, Favipravir, Suramin and Ribavirin, an NS5A inhibitor selected from the group consisting of Velpatasvir, Daclatasvir, Ledipasvir, Elbasvir, Ombitasvir, and Pibrentasvir, or a combination thereof, that is effective to treat the viral infection in the human subject, wherein the NS5A inhibitor inhibits the exonuclease of the coronavirus.

22. A method for treating a viral infection caused by a coronavirus in a human subject afflicted with the viral infection comprising administering to the human subject a therapeutically active dose of an RdRp inhibitor selected from the group consisting of Sofosbuvir, Remdesivir, Favipravir, Suramin and Ribavirin, an NS5A inhibitor selected from the group consisting of Velpatasvir, Daclatasvir, Ledipasvir, Elbasvir, Ombitasvir, and Pibrentasvir, or a combination thereof, that is effective to treat the viral infection in the human subject, wherein the NS5A inhibitor inhibits both the exonuclease and the polymerase activities of the coronavirus.

23. A method for treating a viral infection caused by a coronavirus in a human subject afflicted with the viral infection comprising administering to the human subject a therapeutically active dose of Sofosbuvir, an NS5A inhibitor selected from the group consisting of Velpatasvir, Daclatasvir, Ledipasvir, Elbasvir, Ombitasvir, and Pibrentasvir, or a combination thereof, that is effective to treat the viral infection in the human subject, wherein the NS5A inhibitor inhibits the exonuclease of the coronavirus.

24. A method for treating a viral infection caused by a coronavirus in a human subject afflicted with the viral infection comprising administering to the human subject a therapeutically active dose of Sofosbuvir, an NS5A inhibitor selected from the group consisting of Velpatasvir, Daclatasvir, Ledipasvir, Elbasvir, Ombitasvir, and Pibrentasvir, or a combination thereof, that is effective to treat the viral infection in the human subject, wherein the NS5A inhibitor inhibits both the exonuclease and the polymerase activities of the coronavirus.

25. A method for treating a viral infection caused by a coronavirus in a human subject afflicted with the viral infection comprising administering to the human subject a therapeutically active dose of an RdRp inhibitor selected from the group consisting of Sofosbuvir, Remdesivir, Favipravir, Suramin and Ribavirin, the NS5A inhibitor Velpatasvir, or a combination thereof, that is effective to treat the viral infection in the human subject, wherein Velpatasvir inhibits both the exonuclease and the polymerase activities of the coronavirus.

26. A method for treating a viral infection caused by a coronavirus in a human subject afflicted with the viral infection comprising administering to the human subject a therapeutically active dose of an RdRp inhibitor selected from the group consisting of Sofosbuvir, Remdesivir, Favipravir, Suramin and Ribavirin, the NS5A inhibitor Daclatasvir, or a combination thereof, that is effective to treat the viral infection in the human subject, wherein Daclatasvir inhibits both the exonuclease and the polymerase activities of the coronavirus.

27. A method for treating a viral infection caused by a coronavirus in a human subject afflicted with the viral infection comprising administering to the human subject a therapeutically active dose of an RdRp inhibitor selected from the group consisting of Sofosbuvir, Remdesivir, Favipravir, Suramin and Ribavirin, the NS5A inhibitor Ombitasvir, or a combination thereof, that is effective to treat the viral infection in the human subject, wherein Ombitasvir inhibits the exonuclease of the coronavirus.

28. A method for treating a viral infection caused by a coronavirus in a human subject afflicted with the viral infection comprising administering to the human subject a therapeutically active dose of an RdRp inhibitor selected from the group consisting of Sofosbuvir, Remdesivir, Favipravir, Suramin and Ribavirin, the NS5A inhibitor Pibrentasvir, or a combination thereof, that is effective to treat the viral infection in the human subject, wherein Pibrentasvir inhibits the exonuclease of the coronavirus.

29. A method for treating a viral infection caused by a coronavirus in a human subject afflicted with the viral infection comprising administering to the human subject a therapeutically active dose of Sofosbuvir, Velpatasvir and Remdesivir that is effective to treat the viral infection in the human subject, wherein Velpatasvir inhibits both the exonuclease and the polymerase activities of the coronavirus.

30. A method for treating a viral infection caused by a coronavirus in a human subject afflicted with the viral infection comprising administering to the human subject a therapeutically active dose of Sofosbuvir, Daclatasvir and Remdesivir that is effective to treat the viral infection in the human subject, wherein Daclatasvir inhibits both the exonuclease and the polymerase activities of the coronavirus.

31. A method for treating a viral infection caused by a coronavirus in a human subject afflicted with the viral infection comprising administering to the human subject a therapeutically active dose of an RdRp inhibitor selected from the group consisting of Sofosbuvir, Remdesivir, Favipravir, Suramin and Ribavirin, an exonuclease inhibitor Raltegravir, Ebselen, Ritonavir and Liponavir, or a combination thereof, that is effective to treat the viral infection in the human subject.

32. A method for treating a viral infection caused by a coronavirus in a human subject afflicted with the viral infection comprising administering to the human subject a therapeutically active dose of the RdRp inhibitor Sofosbuvir, an exonuclease inhibitor selected from the group consisting of Raltegravir, Ebselen, Ritonavir and Liponavir, or a combination thereof, that is effective to treat the viral infection in the human subject.

33. A method for treating a viral infection caused by a coronavirus in a human subject afflicted with the viral infection comprising administering to the human subject a therapeutically active dose of the RdRp inhibitor Sofosbuvir, an exonuclease inhibitor selected from the group consisting of Ebselen, Ritonavir and Liponavir, or a combination thereof, that is effective to treat the viral infection in the human subject.

34. A method for treating a viral infection caused by a coronavirus in a human subject afflicted with the viral infection comprising administering to the human subject a therapeutically active dose of an RdRp inhibitor selected from the group consisting of Sofosbuvir, Remdesivir, Favipravir, Suramin and Ribavirin, the exonuclease inhibitor Ritonavir and Lopinavir, or a combination thereof, that is effective to treat the viral infection in the human subject.

35. A method for treating a viral infection caused by a coronavirus in a human subject afflicted with the viral infection comprising administering to the human subject a therapeutically active dose of the RdRp inhibitor Sofosbuvir, an exonuclease inhibitor selected from the group consisting of Ritonavir and Liponavir, or a combination thereof, that is effective to treat the viral infection in the human subject.

36. A method for treating a viral infection caused by a coronavirus in a human subject afflicted with the viral infection comprising administering to the human subject a therapeutically active dose of an RdRp inhibitor selected from the group consisting of Sofosbuvir, Remdesivir, Favipravir, Suramin and Ribavirin, an exonuclease inhibitor selected from the group consisting of NS5A inhibitors, Ritonavir, Lopinavir, Ebselen and Elvitegravir, a helicase inhibitor Ranitidine bismuth citrate, or a combination thereof, that is effective to treat the viral infection in the human subject.

37. A method for treating a viral infection caused by a coronavirus in a human subject afflicted with the viral infection comprising administering to the human subject a therapeutically active dose of an RdRp inhibitor selected from the group consisting of Sofosbuvir, Remdesivir, Favipravir, Suramin and Ribavirin, a helicase inhibitor Ranitidine bismuth citrate, or a combination thereof, that is effective to treat the viral infection in the human subject.

38. A method for treating a viral infection caused by a coronavirus in a human subject afflicted with the viral infection comprising administering to the human subject a therapeutically active dose of the RdRp inhibitor Sofosbuvir, the helicase inhibitor Ranitidine bismuth citrate, or a combination thereof, that is effective to treat the viral infection in the human subject.

39. A method for treating a viral infection caused by a coronavirus in a human subject afflicted with the viral infection comprising administering to the human subject a therapeutically active dose of an RdRp inhibitor selected from the group consisting of Sofosbuvir, Remdesivir, Favipravir, Suramin and Ribavirin, an NS5A inhibitor selected from the group consisting of Velpatasvir, Daclatasvir, Ledipasvir, Elbasvir, Ombitasvir and Pibrentasvir, an NS3/4a protease inhibitor selected from the group consisting of Grazoprevir, Voxilaprevir, Paritaprevir, Glecaprevir, Danoprevir and Telaprevir, or a combination thereof, that is effective to treat the viral infection in the human subject, wherein the NS5A inhibitor inhibits the exonuclease of the coronavirus.

40. A method for treating a viral infection caused by a coronavirus in a human subject afflicted with the viral infection comprising administering to the human subject a therapeutically active dose of the RdRp inhibitor Sofosbuvir, an NS5A inhibitor selected from the group consisting of Velpatasvir, Daclatasvir, Ledipasvir, Elbasvir, Ombitasvir and Pibrentasvir, an NS3/4a protease inhibitor selected from the group consisting of Grazoprevir, Voxilaprevir, Paritaprevir, Glecaprevir, Danoprevir and Telaprevir, or a combination thereof, that is effective to treat the viral infection in the human subject, wherein the NS5A inhibitor inhibits the exonuclease of the coronavirus.

41. A method for treating a viral infection caused by a coronavirus in a human subject afflicted with the viral infection comprising administering to the human subject a therapeutically active dose of an RdRp inhibitor selected from the group consisting of Sofosbuvir, Remdesivir, Favipravir, Suramin and Ribavirin, an NS5A inhibitor selected from the group consisting of Velpatasvir, Daclatasvir, Ledipasvir, Elbasvir, Ombitasvir and Pibrentasvir, the NS3/4a protease inhibitor Voxilaprevir, or a combination thereof, that is effective to treat the viral infection in the human subject, wherein the NS5A inhibitor inhibits the exonuclease of the coronavirus.

42. A method for treating a viral infection caused by a coronavirus in a human subject afflicted with the viral infection comprising administering to the human subject a therapeutically active dose of the RdRp inhibitor Sofosbuvir, the NS5A inhibitor Velpatasvir, and the protease inhibitor Atazanavir, that is effective to treat the viral infection in the human subject.

43. A method for treating a viral infection caused by a coronavirus in a human subject afflicted with the viral infection comprising administering to the human subject a therapeutically active dose of an RdRp inhibitor selected from the group consisting of Sofosbuvir, Remdesivir, Favipravir, Suramin and Ribavirin, an NS5A inhibitor selected from the group consisting of Velpatasvir, Daclatasvir, Ledipasvir, Elbasvir, Ombitasvir and Pibrentasvir, an HIV integrase inhibitor selected from the group consisting of Elvitegravir and Raltegravir, or a combination thereof, that is effective to treat the viral infection in the human subject, wherein the NS5A inhibitor and the Elvitegravir and Raltegravir inhibit the exonuclease of the coronavirus.

44. A method for treating a viral infection caused by a coronavirus in a human subject afflicted with the viral infection comprising administering to the human subject a therapeutically active dose of the RdRp inhibitor Sofosbuvir, an NS5A inhibitor selected from the group consisting of Velpatasvir, Daclatasvir, Ledipasvir, Elbasvir, Ombitasvir and Pibrentasvir, an NS3/4a protease inhibitor selected from the group consisting of Grazoprevir, Voxilaprevir, Paritaprevir, Glecaprevir, Danoprevir and Telaprevir, an HIV integrase inhibitor selected from the group consisting of Elvitegravir and Raltegravir, or a combination thereof, that is effective to treat the viral infection in the human subject, wherein the NS5A inhibitor and the Elvitegravir and Raltegravir inhibit the exonuclease of the coronavirus.

45. A method for treating a viral infection caused by a coronavirus in a human subject afflicted with the viral infection comprising administering to the human subject therapeutically active doses of four drugs, one each derived from each one of the four following classes: an RdRp inhibitor, an NS5A inhibitor, an exonuclease inhibitor, an HIV integrase inhibitor, a helicase inhibitor, and an ns3/4a protease inhibitor, wherein the NS5A inhibitor inhibits the exonuclease of the coronavirus.

46. A method for treating a viral infection caused by a coronavirus in a human subject afflicted with the viral infection comprising administering to the human subject therapeutically active doses of three drugs, one each derived from each one of the three following classes: an RdRp inhibitor, an NS5A inhibitor, an exonuclease inhibitor, an HIV integrase inhibitor, a helicase inhibitor, and an ns3/4a protease inhibitor, wherein the NS5A inhibitor inhibits the exonuclease of the coronavirus.

47. A method for treating a viral infection caused by a coronavirus in a human subject afflicted with the viral infection comprising administering to the human subject therapeutically active doses of three drugs, two derived from one of the following classes and the other one derived from a different one of the following classes: an RdRp inhibitor, an NS5A inhibitor, an exonuclease inhibitor, an HIV integrase inhibitor, a helicase inhibitor, and an ns3/4a protease inhibitor, wherein the NS5A inhibitor inhibits the exonuclease of the coronavirus.

48. The method of any one of claims 21 - 47, wherein the coronavirus is SARS-CoV-2 or a strain that causes SARS or MERS.

49. The method of any one of claims 21 - 47, wherein the coronavirus is SARS-CoV-2.

50. A method for treating a viral infection caused by a coronavirus in a human subject afflicted with the viral infection comprising administering to the human subject therapeutically active doses of the polymerase inhibitor Sofosbuvir, the exonuclease inhibitor Ombitasvir, and a hepatitis C virus NS5A inhibitor selected from the group consisting of Daclatasvir, Velpatasvir and Elbasvir.

51. A composition for the treatment of viral infection caused by coronaviruses, hepatitis C virus, hepatitis C virus, Marburg virus, Ebola virus or influenza virus comprising one or more compounds selected from the group consisting of: 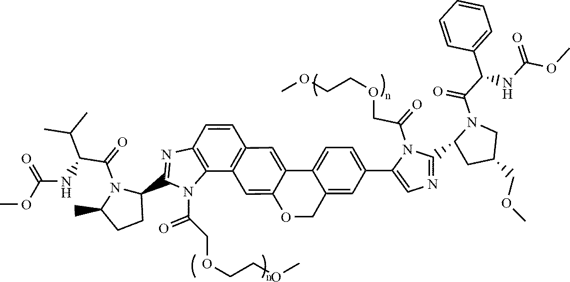 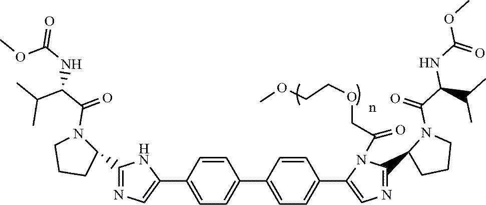
and 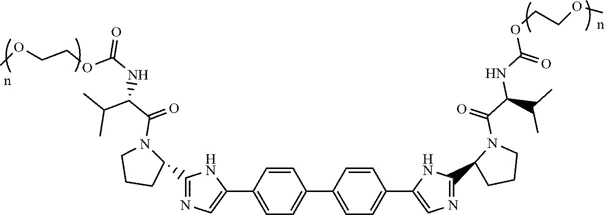
.

52. A composition for the treatment of viral infection caused by one or more viruses selected from the group consisting of coronaviruses and hepatitis C virus comprising one or more compounds selected from the group consisting of: 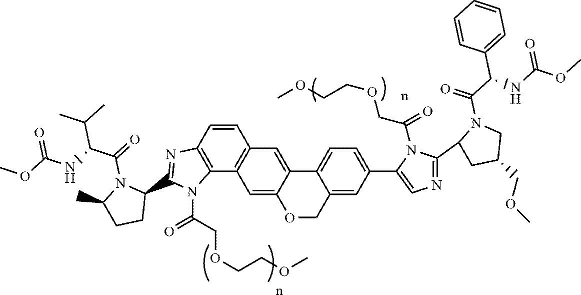 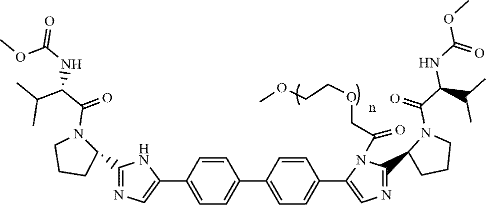
and 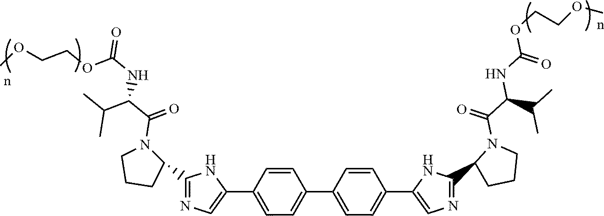
.

53. The composition of any one of claims 51 and 52, wherein the coronaviruses include SARS-CoV-2 and the strains causing SARS and MERS.
